(19) United States
(12) Patent Application Publication
(10) Pub. No.: US 2020/0011873 A1
(43) Pub. Date: Jan. 9, 2020

(54) LEVERAGING SEQUENCE-BASED FECAL MICROBIAL COMMUNITY SURVEY DATA TO IDENTIFY A COMPOSITE BIOMARKER FOR COLORECTAL CANCER

(71) Applicants: Second Genome, Inc., San Francisco, CA (US); Baylor College of Medicine, Houston, TX (US)

(72) Inventors: Todd Zachary Desantis, Livermore, CA (US); Thomas Weinmaier, San Francisco, CA (US); Manasi Sanjay Shah, Santa Clara, CA (US); Emily Brooke Hollister-Branton, Houston, TX (US)

(21) Appl. No.: 16/495,035

(22) PCT Filed: Mar. 16, 2018
(86) PCT No.: PCT/US2018/022862

§ 371 Date: Sep. 17, 2019

Related U.S. Application Data
(60) Provisional application No. 62/472,863, filed on Mar. 17, 2017.

(51) Int. Cl. G01N 33/574; G16B 30/00; G16B 50/00; G16B 25/00; C12Q 1/6886; C12Q 1/6816

(52) U.S. Cl. CPC G01N 33/57446; G16B 30/00; C12Q 1/6816; G16B 25/00; C12Q 1/6886; G16B 50/00

(57) ABSTRACT

The present disclosure provides fecal microbial markers for diagnosing colorectal cancer and colorectal adenoma. The present disclosure also provides methods for diagnosing colorectal cancer and colorectal adenoma using these intestinal microbial markers.

### CROSS-REFERENCE TO RELATED APPLICATIONS

The present application claims the benefit of priority to U.S. Provisional Application No. 62/472,863, filed on Mar. 17, 2017, the contents of which are hereby incorporated by reference in their entirety.

### FIELD OF THE DISCLOSURE

The present disclosure relates to the use of fecal microbiome as a non-invasive biomarker for diagnosing colorectal cancer (CRC) and colorectal adenoma (CRA) and for detecting the transition from adenoma to carcinoma. In particular, the present disclosure relates to the use of 16S rRNA sequences from fecal microorganisms as a marker for diagnosing CRC and CRA.

### DESCRIPTION OF THE TEXT FILE SUBMITTED ELECTRONICALLY

The contents of the text file submitted electronically herewith are incorporated herein by reference in their entirely: A computer readable format copy of the Sequence Listing (filename: SEGE_002_01WO_SegList_ST25, recorded Feb. 18, 2018, file size 315 kilobytes).

### BACKGROUND

Colorectal cancer (CRC) is the third most incident cancer globally and second leading cause of cancer-associated mortality in the United States in men and women combined. [1] Survival exceeds 90% if the cancer is detected at an early, localized stage, but this decreases to 13% with advanced metastatic disease. [2-4] Despite this, adherence to screening recommendations is limited. Greater than 30% of individuals from high risk groups (i.e age ≥50) report never having been screened for CRC. [5]

Colonoscopy, which is invasive, expensive, and fails to address interval cancers (i.e., CRC diagnosed within 6-36 months following a screening colonoscopy) represents the most commonly employed screening method. [5, 6] Home-based fecal occult blood tests (FORT) are used less frequently, owing to perceptions that they are not effective in reducing cancer-associated mortality. [5] FOBT also has low sensitivity in detecting pre-cancerous lesions or colorectal adenoma (CRA). [7]

Cologuard is a newer multi-target stool DNA test. Although it has high sensitivity for detecting CRC, its sensitivity for detecting non-advanced CRA is low, it is more expensive than FOBT, and coverage by insurers varies. [8, 9]

The shortcomings of current screening methods highlight the need for a sensitive, non-invasive diagnostic test for CRC and pre-cancerous lesions, as such a test might increase patient screening rates.

Most CRC and CRA cases are sporadic in nature (i.e., no genetic pattern of inheritance), hence environmental factors such as the gut microbiome have been extensively studied to identify ‘signals’ reflecting the disease. [10-17] The 16S ribosomal RNA (rRNA) gene (rDNA) is a ribosomal component that is conserved in all bacteria, and it contains variable sequences that confer species specificity. Thus, DNA sequencing that targets hypervariabie regions within small ribosomal-subunit RNA genes, especially 16S rRNA genes has made it possible to characterize the biodiversity of the microbiota, Although a number of studies have analyzed the association between the gut microbiome and CRC or CRA, a unifying microbial signature associated with CRC and pre-cancerous CRA has not been defined. While some concordance exists with respect to reported CRC-associated taxa (e.g., Fusobacierium nucleatum, Peptostreptococcus sp., and Polphyromonas sp.), a consistent signal for CRC has not been established. [10, 11, 18, 19] Reported studies have relied on the assessment of a single prokaryotic taxonomic biomarker, the 16S ribosomal RNA (rRNA) gene, which, in theory, would allow the studies to be directly comparable with one another. However, varying experimental methods, 16S rRNA gene target region, sequencing platform, informatics techniques, and demography have limited direct comparability.

Consequently, there is a need for the development of more accurate microbial markers that would indicate the risk of developing CRC or CRA or the presence of CRC or CRA.

### SUMMARY OF THE DISCLOSURE

The present disclosure provides fecal microbial markers for diagnosing colorectal cancer (CRC) or colorectal adenoma (CRA) and methods of using them. The methods of the present disclosure comprise analyzing an intestinal sample from a subject to determine an intestinal microbial profile for the subject and diagnosing the subject as having or not having CRC or CRA.

In some embodiments, the method comprises obtaining an intestinal sample from the subject (“test sample”) and processing the intestinal sample to identify one or more microorganisms and/or operational taxonomic units (OTUs) in the sample.

In some embodiments, the intestinal sample is a stool sample.

In some embodiments, the one or more OTUs comprises a bacterial family, a bacterial genus, a bacterial species, a bacterial strain, or a combination thereof.

In some embodiments, the step of analyzing comprises quantitating the levels of microorganisms and/or OTUs in the intestinal sample. In other embodiments, the step of analyzing comprises comparing the levels of microorganisms and/or OTUs in the intestinal sample with the levels of microorganisms and/or OTUs in a control sample. In still other embodiments, the control sample is obtained from one or more healthy individuals, wherein the healthy individuals are the same species as the subject.

In some embodiments, an increase in the levels of the one or more microorganisms and/or OTUs is indicative of CRC or CRA in the subject. In other embodiments, the increase of the one or more microorganisms and/or OTUs is indicative of CRC. In still other embodiments, a decrease in the levels of the one or more microorganisms and/or OTUs is indicative CRC or CRA in the subject.

In some embodiments, the method comprises diagnosing the subject as having CRC or CRA or as at risk of developing CRC or CRA when the step of analyzing detects the presence in the intestinal sample of 2, 3, 4, 5, 6, 7, 8, 9, 10, 11, 12, 13, 14, 15, 16, 17, 18 19, 20 or 21 of the OTU Identifiers listed in Table 1. In other embodiments, the level of 4, 0.5, 6, 7, 8, 9, 10, 11, 12, 13, 14, 15, 16, 17, 18 19, 20 or 21 of the OTU Identifiers listed in Table 1 is each increased relative to a control sample. In yet other embodiments, the level of 4, 5, 6, 7, 8, 9, 10, 11, 12, 13, 14, 15, 16, 17, 18 19, 20 or 21 of the OTU Identifiers listed in Table 1 is each increased relative to a control sample by at least 2-fold, 4-fold, 5-fold or 10-fold. In still other embodiments, the subject is diagnosed as having CRC or CRA or is at the risk of developing CRC or CRA when the level of 4, 5, 6, 7, 8, 9, 10, 11, 12, 13, 14, 15, 16, 17, 18 19, 2.0 or 21 of the OTU Identifiers listed in Table 1 in the biological sample is each increased by at least about 1.0 fold, 1.1 fold, 1.2 fold, 1.3 fold, 1.4 fold, or 1.5 fold on the log₂ fold-change scale, relative to the control sample. In yet other embodiments, the control intestinal sample is an intestinal sample from at least 5, 10, 15, 20, 25, 30, 40 or 50 healthy individuals. In still other embodiments, the control sample is from a healthy individual which is the same species as the subject.

In some embodiments, the method comprises diagnosing the subject as having CRC or CRA or as at risk of developing CRC or CRA when the step of analyzing detects the presence in the intestinal sample of at least one or more OTU Identifiers, wherein the one or more OTU Identifiers comprises OTU1167 and OTU3191. In other embodiments, the one or more OTU Identifiers further comprises OTU1044. In other embodiments, the one or more OTU Identifiers further comprises OTU2573. In other embodiments, the one or more OTU Identifiers further comprises OTU1.873. In other embodiments, the one or more OTU Identifiers further comprises OTU1169. In other embodiments, the one or more OTU Identifiers further comprises OTU2790. In other embodiments, the one or more OTU Identifiers further comprises OTU2589. In other embodiments, the one or more OTU Identifiers further comprises OTU2910. In other embodiments, the one or more OTU Identifiers further comprises OTU3364. In other embodiments, the one or more OTU Identifiers further comprises OTU2049. In other embodiments, the one or more OTU Identifiers further comprises OTU2703. In other embodiments, the one or more OTU Identifiers further comprises OTU295. In other embodiments, the one or more OTU Identifiers further comprises OTU567. In other embodiments, the one or more OTU Identifiers further comprises OTU569. In other embodiments, the one or more OTU Identifiers further comprises OTU969. In other embodiments, the one or more OTU Identifiers further comprises OTU1255. In other embodiments, the one or more OTU Identifiers further comprises OTU1926. In other embodiments, the one or more OTU Identifiers further comprises OTU2405. In other embodiments, the one or more OTU Identifiers further comprises OTU2691.

In some embodiments, the one or more OTU Identifiers comprises OTU1.1.67, OTU3191, OTU2573, OTU1044, OTU1567, and OTU1873. In other embodiments, the one or more OTU Identifiers comprises OTU1167, OTU2790, OTU3191, and OTU1044.

In some embodiments, the step of detecting the presence of the one or more OTU Identifiers comprises detecting an increase in the one or more OTU Identifiers relative to the levels of the one or more OTU Identifiers in a control sample. In yet other embodiments, the control sample is an intestinal sample from one or more healthy individuals. In still other embodiments, the control sample is an intestinal sample from at least 5, 10, 15, 20, 25, 30, 40 or 50 individuals. In yet other embodiments the control sample is from an individual which is the same species as the subject. In still other embodiments, the intestinal sample is a stool sample.

In another embodiment, the subject is diagnosed as having CRC or CRA or is at the risk of developing CRC or CRA when the level of the one or more OTUs in the biological sample is increased by at least about 1.0 fold, 1.1 fold, 1.2 fold, 1.3 fold, 1.4 fold, or 1.5 fold on the log₂ fold-change scale, relative to the control sample.

The methods of the present disclosure comprise obtaining an intestinal sample (e.g. a stool sample) from a subject (“test sample”); processing the intestinal sample to extract and/or sequence microbial nucleic acids; and analyzing the microbial nucleic acids to identify and quantitate the levels of microorganisms and/or OTUs in the intestinal sample. In some embodiments, the microbial nucleic acid is DNA. In other embodiments, the microbial nucleic acid is RNA. In one embodiment, the test sample is processed to extract and sequence the 16S rRNA gene (rDNA) of microorganisms present in the sample.

In some embodiments, the step of analyzing the microbial nucleic acid comprises analyzing 16S rRNA sequences. In other embodiments, the step of analyzing comprises analyzing one or more hypervariable regions of the 16S rRNA selected from V1, V2, V3, V4, V5, V6, V7, V8 and V9.

In some embodiments, the step of analyzing the microbial nucleic acid comprises using a nucleic acid amplification technique. In some embodiments, the amplification technique is a real time polymerase chain reaction (PCR) or reverse transcription PCR.

In some embodiments, the step of analyzing the microbial nucleic acid comprises nucleic acid sequencing. In other embodiments, the nucleic acid sequencing comprises next-generation sequencing (NGS).

In some embodiments, the step of analyzing the microbial nucleic acid comprises using a nucleic acid microarray.

In some embodiments, the step of analyzing the microbial nucleic acid comprises performing an assay that comprises hybridizing one or more oligonucleotides to one or more nucleic acids represented in an OTU Identifier in Table 1. In other embodiments, the one or more oligonucleotides Which hybridize to the one or more nucleic acids represented in an OTU Identifier comprise oligonucleotides that specifically hybridize to: at least one each of SEQ ID NOS:641-647 (OTU1167), at least one each of SEQ ID NOS:291-513 (OTU3191), at least one each of SEQ ID NOS:191-248 (OTU2790), at least one each of SEQ ID NOS:113-149 (OTU2589), at least one each of SEQ ID NOS:249-259 (OTU2910), at least one each of SEQ NOS:514-546 (OTU3364), at least one each of SEQ ID NOS:26-42 (OTU1169), at least one each of SEQ ID NOS:648-654 (OTU1873), at least one each of SEQ ID NOS:92-98 (OTU2049), at least one each of SEQ II) NOS:8-14 (OTU2573), at least one each of SEQ ID NOS:1-7 (OTU2703), at least one each of SEQ ID NOS:260-290 (OTU295), at least one each of SEQ ID NOS:655-660 (OTU567), at least one each of SEQ m NOS:560-587 (OTU569), at least one each of SEQ ID NOS:588-640 (OTU969), at least one each of SEQ ID NOS:15-25 (OTU1044), at least one each of SEQ m NOS:43-49 (OTU1255), at least one each of SEQ ID NOS:50-91 (OTU926), at least one each of SEQ II) NOS:99-112, (OTU2405), at least one each of SEQ ID NOS:150-190 (OTU2691), and at least one each of SEQ ID NOS:547-559 (OTU467). In still other embodiments, the one or more oligonucleotides which hybridize to the one or more nucleic acids represented in an OTU Identifier comprise oligonucleotides that specifically hybridize to: at least one each of SEQ ID NOS:641-647 (OTU1167), at least one each of SEQ ID NOS:291-513 (OTU3191), at least one each of SEQ ID NOS:648-654 (OTU1873), at least one each of SEQ ID NOS:8-14 (OTU2573), at least one each of SEQ ID NOS:655-660 (OTU567), and at least one each of SEQ ID NOS:15-25 (OTU1044). In yet other embodiments, the one or more oligonucleotides which hybridize to the one or more nucleic acids represented in an OTU Identifier comprise oligonucleotides that specifically hybridize to: at least one each of SEQ ID NOS:641-647 (OTU1167), at least one each of SEQ ID NOS:291-513 (OTU3191), at least one each of SEQ ID NOS:191-248 (OTU2790), at least one each of SEQ ID NOS:8-14 (OTU2573), and at least one each of SEQ ID NOS:15-25 (OTU1044). In some embodiments, each of the one or more oligonucleotides has a length of about 10 to 50 nucleotides; 10 to 40 nucleotides, 10 to 30 nucleotides, 10 to 20 nucleotides, 15 to 40 nucleotides, 15 to 30 nucleotides; 15 to 25 nucleotides, 20 to 40 nucleotides, 25 to 40 nucleotides, 20 to 30 nucleotides, 10 to 25 nucleotides, or 5 to 15 nucleotides.

In some embodiments, the method of analyzing the microbial nucleic acid comprises performing Strain Select-UPARSE (SS-UP) to determine the level of one or more OTU Identifiers. In other embodiments, the step of analyzing the 165 rRNA gene sequence data using SS-UP provides a strain-level resolution of microorganisms and/or OTUs.

In some embodiments, the present disclosure provides a method for detecting the level of one or more microorganisms and/or OTUs in a stool sample of a subject, comprising: obtaining a stool sample from the subject; processing the stool sample to obtain 16S rRNA gene sequences; aligning the 16S rRNA gene sequences against reference sequences in the StrainSelect database; and performing a de novo clustering using SS-UP; and determining the level of one or more microorganisms and/or 01:Us based on the de novo clustering; wherein the one or more microorganisms and/or OTUs are selected from the group of microorganisms and/or OTUs listed in Table 1.

In some embodiments, the present disclosure provides a method for diagnosing colorectal cancer or colorectal adenoma in a subject, comprising: obtaining a stool sample from the subject; processing the stool sample to analyze 16S rRNA gene sequence data; detecting the level of one or more OTUs in the stool sample comprising analyzing the 16S rRNA gene sequence data; and diagnosing the subject as having CRC or CRA or is at the risk of developing CRC or CRA when the level of one or more OTUs in the stool sample is increased relative to a control sample, wherein the one or more OTUs are selected from the group of OTUs listed in Table 1.

In some embodiments, the method for diagnosing colorectal cancer or colorectal adenoma comprises analyzing the 16S rRNA gene sequence data using Strain Select-UPARSE (SS-UP) to determine the level of one or more OTU Identifiers selected from the group consisting of Ser. No. 01/111,167, OTU3191, OTU2573, OTU1044, OTU567, and OTU1873 or from the group consisting of OTU1167, OTU2790, OTU3191, and OTU1044, wherein the increased level of one or more of these OTU Identifiers in the test stool sample compared to a control sample indicates that the subject is suffering from colorectal cancer or colorectal adenoma or is at the risk of developing colorectal cancer or colorectal adenoma. In other embodiments, the increased level of each of OTU1167, OTU3191, OTU2573, OTU1044, OTU567, and OTU1873 or the increased level of each of OTU1167, OTU2790, OTU3191, and OTU1044 in the test stool sample compared to a control sample indicates that the subject is suffering from colorectal cancer or colorectal adenoma or is at the risk of developing colorectal cancer or colorectal adenoma.

In some embodiments, the method for diagnosing CRC or CRA comprises determining the level of OTU1167 in the test sample, wherein an increase in the level of OTU1167 in the test sample indicates that the subject is suffering from colorectal cancer or colorectal adenoma or is at the risk of developing colorectal cancer or colorectal adenoma.

In some embodiments, the method of analyzing the microbial nucleic acid comprises performing a sequence-specific assay, wherein the sequence-specific assay comprises hybridization of a plurality of oligonucleotides to the microbial nucleic acid sequences of the OTU Identifiers listed in Table 1.

In some embodiments, the sequence-specific assay is a PCR reaction that amplifies, detects and quantitates the levels of each of the sequences within the OTU Identifier. In other embodiments, the assay is a microarray assay that detects and quantitates the levels of each of the sequences within the OTU Identifier.

In some embodiments, the method of analyzing the microbial nucleic acid comprises: extracting microbial DNA from the intestinal sample; amplifying the 16S rRNA gene from the extracted microbial DNA; and sequencing the amplified 16S rRNA gene.

In some embodiments, the sequence-specific assay comprises use of oligonucleotides that hybridize to: at least one each of SEQ ID NOS:641-647 (OTU1167), at least one each of SEQ ID NOS:291-513 (OTU3191), at least one each of SEQ ID NOS:191-248 (OTU2790), at least one each of SEQ ID NOS:113-149 (OTU2589), at least one each of SEQ ID NOS:249-259 (OTU2910), at least one each of SEQ ID NOS:514-546 (OTU3364), at least one each of SEQ ID NOS:26-42 (OTU1169), at least one each of SEQ ID NOS:648-654 (OTU1873), at least one each of SEQ ID NOS:92-98 (OTU2049), at least one each of SEQ ID NOS:8-14 (OTU2573), at least one each of SEQ ID NOS:1-7 (OTU2703), at least one each of SEQ ID NOS:260-290 (OTU295), at least one each of SEQ ID NOS:655-660 (OTU567), at least one each of SEQ. ID NOS:560-587 (OTU569), at least one each of SEQ ID NOS:588-640 (OTU969), at least one each of SEQ ID NOS:15-25 (OTU1044), at least one each of SEQ ID NOS:43-49 (OTU1255), at least one each of SEQ ID NOS:50-91 (OTU1926), at least one each of SEQ ID NOS:99-112, (OTU2405), at least one each of SEQ ID NOS:150-190 (OTU2691), and at least one each of SEQ ID NOS:547-559 (OTU467). In other embodiments, the one or more oligonucleotides which hybridize to the one or more nucleic acids represented in an OTUIdentifier comprise oligonucleotides that hybridize to: at least one each of SEQ II) NOS:641-647 (OTU1167), at least one each of SEQ ID NOS:291-513 (OTU3191), at least one each of SEQ ID NOS:648-654 (OTU1873), at least one each of SEQ ID NOS:8-14 (OTU2573), at least one each of SEQ II) NOS:655-660 (OTU567), and at least one each of SEQ ID NOS:15-25 (OTU1044). In yet other embodiments, the one or more oligonucleotides which hybridize to the one or more nucleic acids represented in an OTU identifier comprise oligonucleotides that hybridize to: at least one each of SEQ ID NOS:641-647 (OTU1167), at least one each of SEQ ID NOS:291-513 (OTU3191), at least one each of SEQ ID NOS:191-248 (OTU32790), at least one each of SEQ ID NOS:8-14 (OTU2573), and at least one each of SEQ II) NOS:15-25 (OTU1044).

In some embodiments, the subject is diagnosed as having CRC or CRA or is at the risk of developing CRC or CRA when the level of one or more OTUs in the intestinal sample is increased by at least about 5%, 10% or 15% relative to the control sample.

In some aspects, a diagnostic tool is provided comprising one or more oligonucleotides which are complementary to at leak one each of SEQ ID NOS:641-647 (OTU1167), at least one each of SEQ ID NOS:291-513 (OTU3191), at least one each of SEQ ID NOS:191-248 (OTU2790), at least one each of SEQ ID NOS:113-149 (OTU2589), at least one each of SEQ ID NOS:249-259 (OTU12910), at least one each of SEQ ID NOS:514-546 (OTU3364), at leak one each of SEQ ID NOS:26-42 (OTU1169), at least one each of SEQ ID NOS:648-654 (OTU1873), at least one each of SEQ ID NOS:92-98 (OTU2049), at leak one each of SEQ ID NOS:8-14 (OTU2573), at least one each of SEQ ID NOS:1-7 (OTU2703), at least one each of SEQ ID NOS:260-290 (OTU295), at least one each of SEQ ID NOS:655-660 (OTU567), at least one each of SEQ ID NOS:560-587 (OTU569), at least one each of SEQ ID NOS:588-640 (OTU969), at least one each of SEQ ID NOS:15-25 (OTU1044), at least one each of SEQ ID NOS:43-49 (OTU1255), at least one each of SEQ ID NOS:50-91 (OTU1926), at least one each of SEQ ID NOS:99-112, (OTU2405), at least one each of SEQ ID NOS:150-190 (OTU2691), and at least one each of SEQ ID NOS:547-559 (OTU467). In other embodiments, the one or more oligonucleotides are complementary to: at least one each of SEQ ID NOS:641-647 (OTU1167), at least one each of SEQ ID NOS:291-513 (OTU3191), at least one each of SEQ ID NOS:648-654 (OTU1873), at least one each of SEQ ID NOS:8-14 (OTU2573), at least one each of SEQ ID NOS:655-660 (OTU567), and at least one each of SEQ ID NOS:15-25 (OTU1044). In yet other embodiments, the one or more oligonucleotides are complementary to: at least one each of SEQ ID NOS:641-647 (OTU1167), at least one each of SEQ ID NOS:291-513 (OTU3191), at least one each of SEQ ID NOS:191-248 (OTU2790), at least one each of SEQ ID NOS:8-14 (OTU2573), and at least one each of SEQ ID NOS:15-25 (OTU1044). In some embodiments, the sequence of each of the one or more oligonucleotides is 99% or 100% identical to the complement of the at least one OUT sequence. In some embodiments, the diagnostic composition is a microarray. In other embodiments, the diagnostic composition is a kit which further comprises reagents for performing polymerase chain reactions for detection of one or more OTUs of the present disclosure.

### BRIEF DESCRIPTION OF THE DRAWINGS

FIG. 1 shows a flow chart for the QIIME-CR and SS-UP analysis of the selected studies.

FIG. 2 shows forest plot of selected SS-UP (FIG. 2A) and QIIME-CR OTUs (FIG. 2B). The plots depict per-study and adjusted REM log₂fold change across all studies for OTUs that were detected in ≥5 studies. All OTUs depicted here had an REM FDR <0.1 and the commonly reported Fusobacterium included as well. The length of the error bar depicts the 95% confidence intervals, and the size of point indicates the precision of the point estimate for individual studies (1/(95% CI Upper Bound−95% CI lower bound). The RE-model point size was fixed. Blank values indicate that sequences for that specific OTU were not detected in that particular study. Taxonomic identities presented in FIG. 2A are genus, species, strain (or OTU ID if strain is unclassified) for SS-UP and phylum, genus, species (or OTU ID if species in unclassified) sequence for QIIME-CR in FIG. 2B.

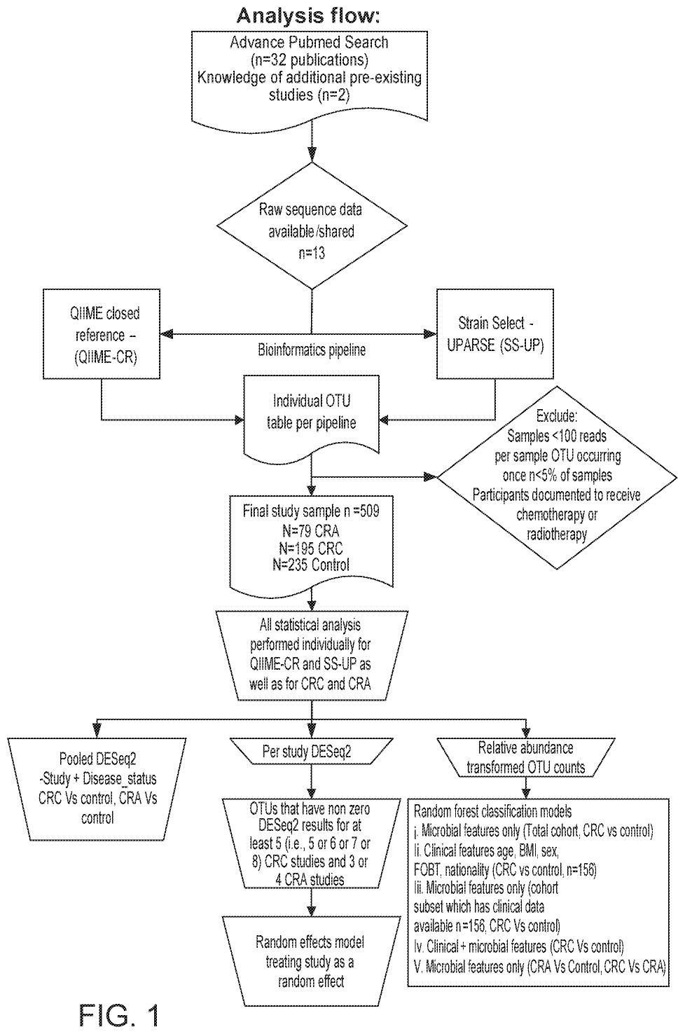

### DETAILED DESCRIPTION

### Definitions

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art. Thus, while the following terms are believed to be well understood by one of ordinary skill in the art, the following definitions are set forth to facilitate explanation of the presently disclosed subject matter.

Throughout this specification, the word “comprise” or variations such as “comprises” or “comprising” will be understood to imply the inclusion of a stated component, or group of components, but not the exclusion of any other components, or group of components.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The term “including” is used to mean “including but not limited to.” “Including” and “including but not limited to” are used interchangeably.

The term “about” when immediately preceding a numerical value means a range of plus or minus 5% or 10% of that value, unless the context of the disclosure indicates otherwise, or is inconsistent with such an interpretation.

The terms “subject,” “patient,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). In certain embodiments, the terms refer to a human patient. In some embodiments, the terms refer to a human patient that suffers from a gastrointestinal disorder.

The present disclosure is based, in part, on the discovery of generalizable microbial markers for CRC and CRA when the raw 16S rRNA gene sequence data from multiple fecal microbial studies was analyzed in a consistent manner across all studies.

The present disclosure provides methods for diagnosing CRC and/or CRA based on the presence of one or more operational taxonomic units (OTUs) in the stool sample of a subject. The present disclosure also provides methods for detecting the presence of one or more OTUs in the stool sample of a subject. In some embodiments, the methods of the present disclosure provide a family, genus, species and/or strain level resolution of one or more microorganisms present in the stool sample of the subject.

“Operational taxonomic unit” (OTU, plural OTUs) refers to a terminal leaf in a phylogenetic tree and is defined by a specific genetic sequence and all sequences that share sequence identity to this sequence at the level of family, genus, species or strain. The specific genetic sequence may be the 16S sequence or a portion of the 16S sequence or it may be a functionally conserved housekeeping gene found broadly across the eubacterial kingdom. OTUs share at least 70%, 75%, 80%, 85%, 90%, 95%, 96%, 97%, 98%, or 99% sequence identity. OTUs are frequently defined by comparing sequences between organisms such that sequences with less than 95% sequence identity are not considered to form part of the same OTU, however, in the systems, algorithms and methods described herein, an OTU Identifier can encompass sequences with 0 to 100%, 25% to 100% and 50% to 100%, preferably 70% to 100%, 75% to 100%, 77% to 100%, 80% to 100%, 81% to 100%, 82% to 100%, 83% to 100%, 84%, to 100%, more preferably 85% to 100%, 86% to 100%, 87% to 100%, 88% to 100%, 89% to 100%. 90% to 100%, 91% to 100%. 92% to 100%, 93% to 100%, 94% to 100%, 95% to 100%, 96% to 100%, 97% to 100% 98% to 100% and 99% to 100% sequence identity.

It is understood herein that detection of an OTU or OTU Identifier as described, e.g., in Table 1 below, is equivalent to the detection of an order, family, genus, species or strain of a bacterium and that an OTU as described in Table 1 be representative of one or more bacteria which may or may not have been previously ascribed a genus, species and/or strain name. Accordingly, the present disclosure relates to methods for diagnosing a subject with CRC or CRA based on the presence of microbes (bacteria) in the intestine of the subject based on the detection of one or more OTUs as described herein, wherein each OTU Identifier is defined by one or more nucleic acid sequences (SEQ ID NOS:1-660).

The “V1-V9 regions” of the 16S rRNA refers to the first through ninth hypervariable regions of the 16S rRNA gene that are used for genetic typing of bacterial samples and which are well understood by ordinarily skilled artisan (Woese et al., 1975, Nature, 254:83-86; Fox et al., 1980, Science, 209:457-463). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al. (PNAS 75:4801-4805 (1978)). In some embodiments, at least one of the V1, V2, V3, V4, V5, V6, V7, V8, and V9 regions are used to characterize an OTU. In one embodiment, the V3 and V4 regions are used to characterize an OTU.

An oligonucleotide that “specifically hybridizes” to an OTU polynucleotide as described herein refers to an oligonucleotide with a sufficiently complementary sequence to permit such hybridization to a target (e.g., OTU) nucleotide sequence under pre-determined conditions routinely used in the art (sometimes termed “substantially complementary”). In particular, the term encompasses hybridization of an oligonucleotide with a substantially complementary sequence contained within a single-stranded DNA or RNA molecule of the disclosure, to the substantial exclusion of hybridization of the oligonucleotide with single-stranded nucleic acids of non-complementary sequence. The specific length and sequence of probes and primers will depend on the complexity of the required nucleic acid target, as well as on the reaction conditions such as temperature and ionic strength. In general, the hybridization conditions are to be stringent as known in the art. “Stringent” refers to the condition under which a nucleotide sequence can bind to related or non-specific sequences. For example, high temperature and lower salt increases stringency such that non-specific binding or binding with low melting temperature will dissolve. In some embodiments, an oligonucleotide that is complementary to an OTU polynucleotide is at least 95%, 96%, 97%, 98%, 99% or 100% complementary to the OTU polynucleotide.

In one embodiment, the method for diagnosing colorectal cancer (CRC) or colorectal adenoma (CRA) in a subject comprises: analyzing nucleic acids from a test sample from the subject; detecting the level of one or more microorganisms and/or OTUs in the nucleic acids from the test sample; and diagnosing the subject as having CRC or CRA or is at the risk of developing CRC or CRA when the level of one or more microorganisms and/or OTUs in the test sample is increased relative to a control sample; wherein the one or more microorganisms and/or OTUs are selected from Table 1.

In another embodiment, the method for diagnosing colorectal cancer (CRC) or colorectal adenoma (CRA) in a subject comprises: obtaining a stool sample from the subject; processing the stool sample to obtain 16S rRNA gene sequence data; detecting the level of one or more microorganisms and/or OTUs in the stool sample comprising analyzing the 16S rRNA gene sequence data using SS-UP; and diagnosing the subject as having CRC or CRA or is at the risk of developing CRC or CRA when the level of one or more microorganisms and/or OTUs in the stool sample is increased relative to a control sample; wherein the one or more OTUs are selected from the group of microorganisms listed in Table 1.

### Sample Collection and DNA Extraction

In various embodiments of the method, the biological sample or the test sample can be selected from stool, mucosal biopsy from a site in the gastrointestinal tract, aspirated liquid from a site in the gastrointestinal tract or combinations thereof. In various embodiments of the method, the site in the gastrointestinal tract can be stomach, small intestine, large intestine, anus or combinations thereof. In some embodiments of the method, the site in the gastrointestinal tract can be duodenum, jejunum, ileum, or combinations thereof. Alternatively, the site in the gastrointestinal tract can be cecum, colon, rectum, anus or combinations thereof. Additionally, the site in the gastrointestinal tract can be ascending colon, transverse colon, descending colon, sigmoid flexure, or combinations thereof.

Stool samples are generally collected in standardized containers at home by the subjects. The subjects are requested to store the samples in their home freezer immediately. Frozen samples are delivered to a laboratory and stored in a freezer until use.

Stool samples are thawed on ice and nucleic acid extraction is performed using standard techniques. The nucleic acid extracted may be DNA and/or RNA. In preferred embodiments, the extracted nucleic acid is DNA.

In one embodiment, Qiagen's QIAamp DNA Stool Mini Kit could be used for extracting DNA from the stool sample. In another embodiment, genomic DNA is extracted from each fecal sample by bead-beating extraction and phenol-chloroform purification, as described previously [47]. Extracts are generally treated with DNase-free RNase to eliminate RNA contamination.

The quantity and quality of DNA is determined using standard techniques such as a spectrophotometer, a fluorometer, and gel electrophoresis. For example, Qubit Fluorometer (with the Quant-iTTMdsDNA BR Assay Kit) could be used to determine the amount of DNA. In another embodiment, the amount of DNA can be determined using Fluorescent and Radioisotope Science Imaging Systems FLA-5100 (Fujifilm, Tokyo, Japan).

Integrity and size of DNA is checked using 0.8% (w/v) agarose gel electrophoresis in 0.5 mg/ml ethidium bromide. All DNA samples are stored at −20° C. until further processing.

### Sequencing of Extracted DNA

Various sequencing methods known in the art can be used to obtain the sequence of 16S rRNA gene, i.e., 16S rDNA sequence, from the extracted DNA. Moreover, universal primers can be designed to amplify the V1, V2, V3, V4, V5, V6, V7, V8 and/or V9 hypervariable regions of 16S rRNA genes.

For example, PCR amplification of the V1-V3 region of bacterial 16 S rDNA can be performed using universal primers (27F 5′-AGAGTTTGATCCTGGCTCAG-3′ SEQ ID NO: 661, 533R 5′-TTACCGCGGCTGCTGGCAC-3′ SEQ ID NO: 662) incorporating the FLX Titanium adapters and a sample barcode sequence. The following PCR cycling parameters can be used: 5 min initial denaturation at 95° C.; 25 cycles of denaturation at 95° C. (30 s), annealing at 55° C. (30 s), elongation at 72° C. (30 s); and final extension at 72° C. for 5 min. Three separate PCR reactions of each sample can be pooled for sequencing. The PCR products are separated by 1% agarose gel electrophoresis and purified by using the QIAquick Gel extraction kit (Qiagen). Equal concentrations of amplicons are pooled from each sample. Emulsion PCR and sequencing are performed as described previously [48]. Alternatively, 16S rRNA gene amplicons can be sequenced on a Roche GS FLX 454 sequencer (Genoscreen, Lille, France).

Alternatively, the V3 region of the 16S rRNA gene from each DNA sample can be amplified using the bacterial universal forward primer 5′-NNNNNNNNCCTACGGGAGGCAGCAG-3′ (SEQ ID NO: 663) and the reverse primer 5′-NNNNNNNNATTACCGCGGCTGCT-3′ (SEQ ID NO: 664). The NNNNNNNN is the sample-unique 8-base barcode for sorting of PCR amplicons into different samples, and the underlined text indicates universal bacterial primers for the V3 region of the 16S rRNA gene. The 16S rRNA gene amplicons are then sequenced.

Alternatively, the V3-V4 region of the 16S rRNA gene from each DNA sample can be amplified using the V3F (TACGGRAGGCAGCAG) forward primer (SEQ ID NO: 665) and V4R (GGACTACCAGGGTATCTAAT) (SEQ ID NO: 666) reverse primer to target the V3-V4 region. The 16S rRNA gene amplicons are then sequenced.

The sequencing reads can be filtered according to barcode and primer sequences. The resulting sequences can be further screened and filtered for quality and length. Sequences that are less than 150 nucleotides, contain ambiguous characters, contain over two mismatches to the primers, or contain mononucleotide repeats of over six nucleotides are removed.

Analysis of the 16S rRNA Gene Sequence Data Using SS-UP

Strain Select—UPARSE (SS-UP) (Second Genome, Inc) methodology is used to analyze the 16S rRNA gene sequence data. SS-UP utilizes the StrainSelect database, a collection of high-quality sequence and annotation data derived from bacterial and archaeal strains that can be obtained from an extant culture collection (secondgenome.com/StrainSelect), and conducts de novo clustering of all sequences without strain hits. The SS-UP method is described in “UPARSE: highly accurate OTU sequences from microbial amplicon reads”, Edgar R C, Nat Meth, 2013, 10: 996-8″, which is reference number 34 at the end of this discourse, which is incorporated by reference herein in its entirety.

For performing de novo clustering using SS-UP, paired-end sequenced reads can be merged using USEARCH fastq_mergepairs with default settings except for dataset-specific cutoffs for fastq_minmergelen and fastq_maxmergelen (Tables 3A-3B). All resulting merged sequences are compared against the StrainSelect database using USEARCH's usearch_global. Single-end reads are first quality trimmed from the N-terminal end using PrinSeq-lite [26] and parameters ‘-trim_ns_left 1-trim_ns_right 1-min_len $MIN_LEN-trim_qual_right 20’ (minimal length values per dataset are summarized in Tables 3A-3B) before comparison to StrainSelect using USEARCH's usearch_global.

Distinct strain matches are defined as those with ≥99% identity to a 16S sequence from the closest matching strain and a lesser identity (even by one base) to the second closest matching strain. Those distinct hits are summed per strain and a strain-level OTU abundance table is created. The remaining sequences are filtered by overall read quality using USEARCH's fastc_maxee and a MAX_EE value of 1, length-trimmed to the lower boundary of the 95% interval of the read length distribution (for datasets with an uneven read length distribution length-trimming to the shortest read length is strongly affected by very short reads; the 95% interval is used to compensate for this outlier effect), de-replicated, sorted descending by size and clustered at 97% identity with USEARCH (fastq_filter, derep_fulllength, sortbysize, cluster_otus). USEARCH cluster_otus discards likely chimeras.

De novo OTUs with abundance of less than 3 are discarded as spurious. All sequences that are used in the comparison against StrainSelect but do not end up in a strain OTU can then be mapped to the set of representative consensus sequences (≥97% identity) to generate a de novo OTU abundance table. Representative strain-level OTU sequences and representative de novo OTU sequences are assigned a Greengenes [12] taxonomic classification via mothur's bayesian classifier [28] at 80% confidence the classifier is trained against the Greengenes reference database (e.g. version 13_5) of 16S rRNA gene sequences. Where standard taxonomic names have not been established, a hierarchical taxon identifier is used (for example “97otu15279”). Strain-level OTU abundances and taxonomy-mapped de novo OTU abundances are merged and used for further analysis. The SS-UP approach allows all high-quality sequences to be counted, and the taxonomic classification of the de novo OTUs permits de novo OTUs with conserved taxonomy to be compared across various samples.

Samples with <100 sequences after quality filtering and OTU assignment are excluded from further analysis.

Statistical analysis can be performed using standard tools. For example, the R package phyloseq can be used for determining global community properties such as alpha diversity, beta diversity metrics such as the Bray-Curtis and Jaccard index, principle coordinate scaling of Bray-Curtis dissimilarities, Firmicutes/Bacteroidetes (F/B) ratio and differential abundance analysis. Two-sample permutation t-tests using Monte-Carlo resampling can be used to compare the alpha diversity estimates and F/B ratio across CRC and controls and CRA and controls. Permutational analysis of variance (PERMANOVA) can be used to test whether within group distances were significantly different from between group distances using the adonis function in the vegan package. Multivariate homogeneity of group dispersions can be tested with vegan using the betadisper function. OTUs are considered significantly different if their False Discovery Rate (FDR) adjusted Benjamin Hochberg (BH) p value is <0.1 and estimated log₂-fold change is >1.5 or <−1.5.

Statistical analysis can also be performed using other tools such as SPSS Statistics.

### Diagnostic Methods

In some embodiments, the method for diagnosing colorectal cancer or colorectal adenoma comprises analyzing the fecal 16S rRNA gene sequences using the Strain Select-UPARSE (SS-UP) method for the presence of one or more microorganism or OTUs.

In one embodiment, the SS-UP method comprises aligning the 16S rRNA gene sequences against the reference sequences in the StrainSelect database available at secondgenome.com/StrainSelect and performing a de novo clustering using SS-UP.

In an alternative embodiment, the level of microorganisms and/or 01Us is determined through standard nucleic acid detection and quantitation techniques well known in the art, including but not limited to polymerase chain reaction (PCR) and real time PCR in which forward and reverse primers are designed to hybridize to sequences representative of each OTU Identifier as identified in Table 1 (SEQ ID NOS:1-660) and levels of the reaction products are quantitated. Also included is a method for analyzing RNA levels in which RNA is extracted and reverse transcription is performed for subsequent PCR amplification of 16S rRNA sequences. Methods for detecting levels of microorganisms and/or OTUs in a sample can also include routine microarray analysis in which probes that selectively hybridize directly or indirectly to sequences representative of each OTU Identifier as identified in Table 1 (SEQ ID NOS:1-660) are used to detect and quantitate polynucleotides extracted from a sample.

Hybridization assays such as PCR, qPCR, RT-PCR, and microarray analysis are routinely used in the art and one of skill in the art would understand how to apply these techniques for the analysis and quantitation of the microorganisms and/or OTUs disclosed herein for diagnostic purposes.

When determining levels of microorganisms and/or OTUs using sequence-specific or sequence-selective methods such as PCR and microarray methods, oligonucleotides (e.g., primers and probes) are designed to hybridize to one or more of sequences representative of one or more OTU Identifiers in Table 1. For example, to detect OTL11167 which is represented by 7 sequences (SEQ ID NOS:641-647), PCR can be used to amplify each of SEQ ID NOS:641-647. Alternatively, a microarray can be designed to detect and quantitate each of SEQ NOS:641-647. Accordingly, the detection levels for nucleic acids corresponding to SEQ ID NOS:641-647 in the test samples are compared to the detection levels for nucleic acids corresponding to SEQ ID NOS:641-647 in the healthy control sample(s).

Oligonucleotides that hybridize or anneal to a specified nucleic acid sequence for the purpose of, e.g., PCR and microarray analysis (i.e., a polynucleotide having a sequence of one of SEQ m NOS:1-660) are readily determined using routine methods and/or software, based on the well-understood knowledge of nucleotide base-pairing interaction of one nucleic acid with another nucleic acid that results in the formation of a duplex, triplex, or other higher-ordered structure. The primary interaction is typically nucleotide base specific, e.g. A:T, A:U, and G:C, by Watson-Crick and Hoogsteen-type hydrogen bonding. In certain embodiments, base-stacking and hydrophobic interactions may also contribute to duplex stability. Conditions under which primers anneal to complementary or substantially complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349, 1968. In general, whether such annealing takes place is influenced by, among other things, the length of the complementary portion of the primers and their corresponding primer-binding sites in adapter-modified molecules and/or extension products, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or minor groove binders in the complementary portions of the primers and reporter probes can also influence hybridization conditions. Thus, the preferred annealing conditions will depend upon the particular application. Such conditions, however, can be routinely determined by persons of ordinary skill in the art, without undue experimentation. Typically, annealing conditions are selected to allow the described oligonucleotides to selectively hybridize with a complementary or substantially complementary sequence in their corresponding adapter-modified molecule and/or extension product, but not hybridize to any significant degree to other sequences in the reaction.

Oligonucleotides and variants thereof that “selectively hybridize” to, e.g., a second polynucleotide comprising a sequence of one of SEQ ID NOS:1-660, are understood to be those that under appropriate stringency conditions, anneal with the second nucleotide that comprises a complementary string of nucleotides (for example but not limited to a target flanking sequence or a primer-binding site of an amplicon), but does not anneal to polynucleotides comprising undesired sequences, such as non-target nucleic acids or other primers. Typically, as the reaction temperature increases toward the melting temperature of a particular double-stranded sequence, the relative amount of selective hybridization generally increases and mis-priming generally decreases. Accordingly, a statement that an oligonucleotide hybridizes or selectively hybridizes with another oligonucleotide or polynucleotide encompasses situations where the entirety of at least one of the sequences hybridize to an entire other nucleotide sequence or to a portion of the other nucleotide sequence.

Routine methods are used to adjust detection signals to account for sample amount and number of unique sequences or reactions used for detection of each OTU Identifier in order to calculate the corresponding level of each OTU Identifier in a sample.

In one embodiment, the subject is diagnosed as having colorectal cancer or colorectal adenoma or is diagnosed as at the risk of developing colorectal cancer or colorectal adenoma when the level of one or more microorganisms or OTUs in the test sample obtained from the subject (e.g. a stool sample) is increased relative to a control sample.

A control or a control sample is a sample obtained from a healthy subject. The term “healthy subject” as used herein refers to a subject not suffering from and/or is not at the risk of developing CRC or CRA. In some embodiments, a control sample is obtained by pooling samples from at least 5, 10, 25, or 50 healthy subjects.

In some embodiments, the subject is diagnosed as having colorectal cancer or colorectal adenoma or is diagnosed as at the risk of developing colorectal cancer or colorectal adenoma when the level of one or more microorganisms or OTUs in the test sample is increased by about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, including values and ranges therebetween, relative to a control sample.

In another embodiment, the subject is diagnosed as having colorectal cancer or colorectal adenoma or is diagnosed as at the risk of developing colorectal cancer or colorectal adenoma when the level of one or more microorganisms or OTUs in the test sample is changed by about 1.2 fold on the log 2 fold-change scale, relative to a control sample. The term “change” encompasses an increase or a decrease in the level of microorganisms or OTUs in the test sample compared to a control sample. In some embodiments, the change in the level of one or more microorganisms or OTUs between the test sample and the control sample could be about 1.2 fold, 1.3 fold, 1.4 fold, 1.5 fold, 1.6 fold, 1.7 fold, 1.8 fold, 1.9 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.6 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, or 5 fold, including values and ranges therebetween, on the log₂ fold-change scale, relative to a control sample.

In some embodiments, the subject is diagnosed as having colorectal cancer or colorectal adenoma or is diagnosed as at the risk of developing colorectal cancer or colorectal adenoma when the level of one or more microorganisms or OTUs in the test sample is increased by about 1.2 fold, 1.3 fold, 1.4 fold, 1.5 fold, 1.6 fold, 1.7 fold, 1.8 fold, 1.9 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.6 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, or 5 fold, including values and ranges therebetween, on the log₂ fold-change scale, relative to a control sample.

In some embodiments, the subject is diagnosed as having colorectal cancer or colorectal adenoma or is diagnosed as at the risk of developing colorectal cancer or colorectal adenoma when the level of one or more microorganisms or OTUs in the test sample is decreased by about 1.2 fold, 1.3 fold, 1.4 fold, 1.5 fold, 1.6 fold, 1.7 fold, 1.8 fold, 1.9 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.6 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, or 5 fold, including values and ranges therebetween, on the log₂ fold-change scale, relative to a control sample.

The microorganisms and/or OTUs that could be used as markers for diagnosing CRC or CRA according to the present disclosure are selected from the microorganisms and OTUs listed in Table 1.

TABLE 1

| OTU Identifier | | SEQ |
|---|---|---|
| (# sequences) | Microbial marker | ID NO. |
| OTU1167 (7) | Parvimonas micra ATCC 32770 | 641-647 |
| OTU3191 (223) | Proteobacteria OTU 3191 | 291-513 |
| OTU2790 (58) | Fusobacterium sp. OTU 2790 | 191-248 |
| OTU2589 (37) | Dialister sp, OTU 2589 | 113-149 |
| OTU2910 (11) | Enterococcus sp. OTU 2910 | 249-259 |
| OTU3364 (33) | Akkermansia muciniphila OTU 3364 | 514-546 |
| OTU1169 (17) | Parvimonas sp OTU 1169 | 26-42 |
| OTU1873 (7) | Peptostreptococcus stomatis DSM 17678 | 648-654 |
| OTU2049 (7) | Peptostreptococcus anaerobius OTU2049 | 92-98 |
| OTU2573 (7) | Dialister pneumosintes ATCC 33048 | 8-14 |
| OTU2703(7) | Clostridium spiroforme DSM 1552 | 1-7 |
| OTU295 (31) | Actinobacteria OTU 295 | 260-290 |
| OTU567 (6) | Porphyromonas asaccharolytica DSM 20707 | 655-660 |
| OTU569 (28) | Porphyromonas OTU 569 | 560-587 |
| OTU969 (53) | Lactobacillus OTU 969 | 588-640 |
| OTU1044 (11) | Streptococcus anginosus OTU1044 | 15-25 |
| OTU1255 (7) | Firmicutes OTU1255 | 43-49 |
| OTU1926 (42) | Lachnospira OTU 1926 | 50-91 |
| OTU2405 (14) | Oscillospora OTU 2405 | 99-112 |
| OTU2691 (41) | Eubacterium dolichum OTU 2691 | 150-190 |
| OTU467 (13) | Bacteroides caccae OTU 467 | 547-559 |

In a particular embodiment, the method for diagnosing CRC or CRA in a subject comprises: obtaining a stool sample from the subject; processing the stool sample to obtain 16S rRNA gene sequence data; detecting the level of one or more microorganisms and/or OTUs in the stool sample comprising analyzing the 16S rRNA gene sequence data using Strain Select-UPARSE; and diagnosing the subject as having CRC or CRA or is at the risk of developing CRC or CRA when the level of one or more microorganisms and/or OTUs in the stool sample is increased relative to a control sample; wherein the one or more microorganisms and/or OTUs are selected from the group of microorganisms and or OTUs listed in Table 1.

In another particular embodiment, the method for diagnosing CRC or CRA in a subject comprises: obtaining a stool sample from the subject; processing the stool sample to obtain 165 rRNA gene sequence data; detecting the level of one or more microorganisms and/or OTUs in the stool sample comprising analyzing the 16S rRNA gene sequence data using Strain Select-UPARSE; and diagnosing the subject as having CRC or CRA or is at the risk of developing CRC or CRA when the level of one or more microorganisms and/or OTUs in the stool sample is increased relative to a control sample; wherein the one or more microorganisms and/or OTUs comprise those of OTU identifiers OTU1167, OTU3191, OTU2573, OTU1044, OTU567, and OTU1873.

In another particular embodiment, the method for diagnosing CRC or CRA in a subject comprises: obtaining a stool sample from the subject; processing the stool sample to obtain 16S rRNA gene sequence data; detecting the level of OTU1167, OTU2790, OTU3191 and OTU1044 in the stool sample comprising analyzing the 16S rRNA gene sequence data using Strain Select-UPARSE; and diagnosing the subject as having CRC or CRA or is at the risk of developing CRC or CRA when the level of each of OTU1167, OTU2790, OTU3191 and OTU1044 in the stool sample is increased relative to a control sample.

In one embodiment, the Strain Select-UPARSE method provides a strain-level resolution of the microorganisms present in the patient's stool sample.

In one embodiment, the Strain Select-UPARSE method provides an AUROC (area under receiver operator characteristic curve of at least about 80%, 81%, 82%, 83%, 84%, 85%, 86%, 87%, 88%, 89%, 90%, 91%, 92%, 93%, 94%, or 95%. For example, in one embodiment, the Strain Select-UPARSE method provides an AUROC value of 89.6%. In another embodiment, the Strain Select-UPARSE method provides a diagnostic AUROC value of 91.3%.

The Strain Select-UPARSE method provides a strain-level resolution compared to the species-level resolution provided by QIIME-CR.

The Strain Select-UPARSE method provides an improved AUROC value compared to that of QIIME-CR. For example, in one embodiment, the Strain Select-UPARSE method provides an AUROC value of 80.3% compared to the AUROC value of 76.6% provided by QIIME-CR. In another embodiment; the Strain Select-UPARSE method provides a diagnostic AUROC value of 91.3% compared to the AUROC value of 83.3% for QIIME-CR.

In some embodiments, the level of one or more microorganisms and/or OTUs in the stool sample is detected using the SS-UP method described above.

In some other embodiments, the level of one or more microorganisms and/or ° This in the stool sample can be detected using quantitative PCR (qPCR). For example, microbial DNA is extracted from the stool sample as described above. In a qPCR, the 16S rRNA gene from the extracted DNA is amplified using universal primers described above and simultaneously quantified using a universal probe. In the same qPCR, a probe specific or selective for the microorganisms and/or OTUs of interest can be included to quantitate the level of that microorganism or OTU. For example, a qPCR can include universal primers and a universal probe for the amplification and quantification of total microbial 16S rRNA gene and one or more probes selective for the microorganisms and/or OTUs listed in Table 1, such as, a probe specific or selective for Parvimonas micro ATCC 32770 (OTU Identifier OTU1167, SEQ ID NOS:641-647), a probe specific for Dialisier pneumosinies ATCC 33048 (OTU Identifier OTU2573, SEQ ID NOS:8-14), and so on. The probes selective for the microorganism or OTU helps in quantifying the level of that particular microorganism or OTU.

An additional embodiment is the use of a polynucleotide microarray assay wherein target oligonucleotides which will selectively hybridize to OTU polynucleotides obtained from processing of an intestinal sample.

In other words, detection and quantification of microorganisms and/or OTUs listed in Table 1 can be achieved using routine assays (e.g., quantitative PCR, real time PCR, microarray) which use oligonucleotides which selectively hybridize to one or more sequences for each microorganism/OTU as defined in the SEQ ID NOS. provided in Table 1, i.e., oligonucleotides which are identical to, 90%, 92%, 94%, 95%, 96%, 97%, 98%, 99% or 100% identical along their full length to a portion of a Table 1 SEQ ID NO. for the specified OTU, or the complement thereof.

Moreover, probe-selective based quantitative reactions (e.g., PCR, microarray) can be designed to include all or almost all of the sequences within an OTU Identifier (e.g.; 6 of the 7 or all 7 sequences for OTU1167; 200 of the 223 sequences for OTU3191 or all 223 sequences for OTU3191). Alternatively or additionally, one may include oligonucleotides that hybridize to at least 50%, 60%, 70%, 80%, 90%, 95%, 99% or 100% of the sequences within an OTU Identifier listed in Table 1 to detect and quantitate the levels of the OTU in an intestinal sample.

Accordingly, in some embodiments, the method for diagnosing CRC or CRA in a subject comprises: obtaining a stool sample from the subject; extracting microbial DNA from the stool sample; amplifying 16S rRNA gene from the extracted DNA; quantifying the level of 16S rRNA gene and the level of one or more microorganisms and/or OTUs using qPCR, RT-PCR, or microarray; and diagnosing the subject as having CRC or CRA or is at the risk of developing CRC or CRA when the level of one or more microorganisms and/or OTUs in the stool sample is increased relative to a control sample; wherein the one or more microorganisms and/or OTUs are selected from the group of microorganisms and or OTUs listed in Table 1.

In another particular embodiment, the method for diagnosing CRC or CRA in a subject comprises: obtaining a stool sample from the subject; extracting microbial DNA from the stool sample; amplifying 16S rRNA gene from the extracted DNA; quantifying the level of 16S rRNA gene and the level of one or more microorganisms and/or OTUs using qPCR, RT-PCR, or microarray; and diagnosing the subject as having CRC or CRA or is at the risk of developing CRC or CRA when the level of one or more microorganisms and/or OTUs in the stool sample is increased relative to a control sample; wherein the one or more microorganisms and/or OTUs comprise those of OTU Identifiers OTU1167, OTU3191, OTU2573, OTU1044, OTU567, and OTU1873.

In another particular embodiment, the method for diagnosing CRC or CRA in a subject comprises: obtaining a stool sample from the subject; detecting the level of OTU1167, OTU2790, OTU3191 and OTU1044 in the stool sample; and diagnosing the subject as having CRC or CRA or is at the risk of developing CRC or CRA when the level of each of OTU1167, OTU2790, OTU3191 and OTU1044 in the stool sample is increased relative to a control sample.

In the embodiments using quantitative PCR, the subject can be diagnosed as having colorectal cancer or colorectal adenoma or is at the risk of developing colorectal cancer or colorectal adenoma when the level of one or more microorganisms or OTUs in the test sample is increased by about 2%, 3%, 4%, 5%, 6%, 7%, 8%, 9%; 10%, 11%, 12%, 13%, 14%, 15%, 16%, 17%, 18%, 19%, 20%, 21%, 22%, 23%, 24%, or 25%, including values and ranges therebetween, relative to a control sample.

In another embodiment using quantitative PCR, the subject can be diagnosed as having colorectal cancer or colorectal adenoma or is at the risk of developing colorectal cancer or colorectal adenoma when the level of one or more microorganisms or OTUs in the test sample is changed by about 1.2 fold, 1.3 fold, 1.4 fold, 1.5 fold, 1.6 fold, 1.7 fold, 1.8 fold, 1.9 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.6 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, or 5 fold, including values and ranges therebetween, relative to a control sample.

### Diagnostic Tools

The teachings of this disclosure support a variety of diagnostic tools or devices which can be used to carry out the diagnostic methods described herein. For example, a diagnostic test may include use of PCR reactions, polynucleotide sequencing and/or microarray hybridization to detect the presence and levels of one or more of the OTUs of the present disclosure. Accordingly, any one of these diagnostic tools or devices, e.g., nucleotide microarray, PCR kit, nucleotide sequencing kit, etc., will comprise a set of oligonucleotides which are complementary to the one or more OTUs according to the present disclosure.

Each of the oligonucleotides complementary to the one or more OTUs as described herein can specifically hybridize to its complementary OTU. As used herein, the phrase “specifically hybridize” or “capable of specifically hybridizing” means that a sequence can bind, be double stranded or hybridize substantially or only with a specific nucleotide sequence or a group of specific nucleotide sequences under stringent hybridization conditions when the sequence is present in a complex mixture of DNA or RNA. Generally, it is known that nucleic acids are denatured by elevated temperatures, or reduced concentrations of salts in a buffer containing the nucleic acids. Under low stringent conditions (such as low temperature and/or high salt concentrations), hybrid double strands (for example, DNA:DNA, RNA:RNA or RNA:DNA) are formed as a result of gradual cooling even if the paired sequence is not completely complementary. Therefore, the specificity of the hybridization is reduced under low stringent conditions. On the contrary, under high stringent conditions (for example, high temperature or low salt concentration), it is necessary to keep as little mismatch as possible for proper hybridization.

Those skilled in the art would understand that hybridization conditions can be selected such that an appropriate level of stringency is achieved. In one exemplary embodiment, hybridization is performed under low stringency conditions such as 6×SSPE-T at 37° C. (0.05% Triton X-100) to ascertain thorough hybridization. Thereafter, a wash is performed under high stringent conditions (such as 1×SSPE-T at 37° C.) to remove mismatch hybrid double strands. A serial wash can be performed with increasingly high stringency (for example, 0.25 SSPE-T at 37° C. to 50° C.) until a desired level of hybridization specificity. The specificity of the hybridization can be verified by comparing the hybridization of the sequence with a variety of probable controls (for example, an expression level control, a standardization control, a mismatch control, etc.) with the hybridization of the sequence with a test probe. Various methods for optimization of hybridization conditions are well known to those skilled in the art (for example, see P. Tijssen (Ed) “Laboratory Techniques in Biochemistry and Molecular Biology”, vol. 24; Hybridization With Nucleic Acid Probes, 1993, Elsevier, N.Y.).

This disclosure is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the disclosure.

All patent and non-patent documents referenced throughout this disclosure are incorporated by reference herein in their entirety for all purposes.

### EXAMPLES

### Example 1

To determine if generalizable microbial markers for CRC and CRA could be identified, we accessed the raw 165 rRNA gene sequence data from multiple fecal microbial studies published during the years 2006 to 2016. We analyzed the data using two bioinformatics pipelines, (1) QIIME closed reference (QIIME-CR), a closed-reference OTU assignment approach used in previously published meta-analyses [20-22] and (2) Strain Select UPARSE (SS-UP), a strain specific method that utilized more raw sequence data and offered strain-level resolution in some cases. Additionally, where data was available, we compared our composite microbial markers to the take-home guaiac-based fecal occult blood test (FORT), a non-invasive but imprecise test. [23, 24]

Study Search, Selection, and Inclusion

We performed a systematic PubMed search to identify studies with the terms colorectal cancer, colon cancer, colorectal adenocarcinoma in the title, which included human subjects, and were published within the years 2006-2016. The final detailed search term using PubMed advanced search was ((((((((((bacterial microbiome OR gut microbiome OR microbiota OR microbial)) AND (fecal or feces)) AND (colorectal cancer[Title] OR colon cancer[Title] OR colorectal adenoma[Title] OR adenomatous polyp[Title] or colorectal carcinoma[Title])) AND (“2006/01/01”[PDAT]:“2016/04/01”[PDAT])) AND humans[MeSH Terms]) NOT review[Publication Type]) AND Humans[Mesh])). The manuscript required the terms bacterial microbiome, gut microbiome or microbiota in its main text, the terms colorectal cancer or colorectal adenoma or adenomatous polyp or colorectal carcinoma in the title, included human subjects only and published within the years 2006-2016.

To present an unbiased synthesis of epidemiological studies evaluating associations of the fecal microbiome with CRC, we followed the MOOSE (Meta-analysis of Observational Studies in Epidemiology) checklist of recommendations to identify and include studies for our analysis. [25]. Studies fit our inclusion criteria if they: (i) used 454 or Illumina sequencing for 16S rRNA gene amplicons, (ii) included histologically-confirmed CRC or CRA samples and controls; and (iii) had sequence and associated metadata available publicly or shared by authors by April 2016.

Thirteen studies evaluating fecal microbial associations with CRC were identified by the systematic search described above. The studies varied with respect to DNA extraction method, 16S rRNA gene variable region targeted, sequencing platform, and study characteristics and are summarized in Tables 2A and 2B.

TABLE 2A
Characteristics of fecal studies included in the meta-analysis

Timepoint

| | of bio- | | | | | | | Source | |
|---|---|---|---|---|---|---|---|---|---|
| Study, | specimen | DNA | PCR | | Seq | Seq | | of | Data |
| year | collection | Extraction | Primer | Region | Plat | dir | Samples | data | shared |
| Wang | No medica- | bead- | 331F, | V3 | 454-FLX | F, | CRA-0, | NCBI | ✓ |
| et al, | tion, | beating and | 797R | | | R | CRC-46, | SRA | |
| 2012 | before | phenol- | | | | | Ctrl-56, | | |
| [14] | surgery | chloroform | | | | | Total- | | |
| | | purifica- | | | | | 102 | | |
| | | tion | | | | | | | |
| Chen | No medica- | QIAamp DNA | 27F, | V1-V3 | 454-FLX | F | CRA-0, | NCBI | ✓ |
| et al, | tion, | | 533R | | | | CRC-22, | SRA | |
| 2012 | prior to | | | | | | Ctrl-21, | | |
| [26] | bowel | | | | | | Total- | | |
| | cleanse | | | | | | 43 | | |
| Wu | No anti- | QIAamp DNA | 341F, | V3 | 454-FLX | F, | CRA-0, | NCBI | ✓ |
| et al, | biotics | | 534R | | | R | CRC-19, | SRA | |
| 2013 | for three | | | | | | Ctrl-20, | | |
| [12] | months, | | | | | | Total- | | |
| | timepoint | | | | | | 39 | | |
| | of bio- | | | | | | | | |
| | specimen | | | | | | | | |
| | collection | | | | | | | | |
| | not ex- | | | | | | | | |
| | plicitly | | | | | | | | |
| | mentioned | | | | | | | | |
| Weir | No anti- | MoBio | 515F, | V4 | 454-FLX | F | CRA-0, | ENA | ✓ |
| et al, | biotics | Powersoil | 806R | | | | CRC-7, | | |
| 2013 | for two | | | | | | Ctrl-8, | | |
| [13] | months, | | | | | | Total- | | |
| | prior to | | | | | | 15 | | |
| | colonic | | | | | | | | |
| | resection | | | | | | | | |
| | surgery | | | | | | | | |
| Brim | Home based | QIAamp | Not | V1-V3 | 454- | F | CRA-6, | NCBI | ✓ |
| et al, | bio- | Stool | pro- | | Titanium | | CRC-0, | SRA | |
| 2013 | specimen | DNA ex- | vided | | | | Ctrl-6, | | |
| [29] | collection | traction | | | | | Total- | | |
| | two months | Kit | | | | | 12 | | |
| | after col- | | | | | | | | |
| | onoscopy | | | | | | | | |
| Zackular | Prior to | MoBio | F: | V4 | Illumina- | F, | CRA-30, | ENA | ✓ |
| et al, | curative | Powersoil | GTGCCAG | | MtSeq | R | CRC-30, | | |
| 2014 | surgery, | | CMGCCAGC | | | | Ctrl-30, | | |
| [11] | radiation | | MGCCGCGG | | | | Total- | | |
| | therapy | | TAA | | | | 90 | | |
| | | | R: | | | | | | |
| | | | TAATCTW | | | | | | |
| | | | TGGGVHCA | | | | | | |
| | | | TCAGG | | | | | | |
| | | | (custom) | | | | | | |
| Zeller | Prior to | G'NOME | 515F, | V4 | Illumina- | F, | CRA-13, | Author | ✓ |
| et al, | bowel prep | DNA | 806R | | MtSeq | R | CRC-41, | | |
| 2014: | for col- | | | | | | Ctrl-75, | | |
| | onoscopy | | | | | | Total- | | |
| | and re- | | | | | | 129 | | |
| | section | | | | | | | | |
| | surgery | | | | | | | | |
| Mira- | One week | Macherey- | 27F, | V1-V3 | 454-FLX | F | CRA-11, | MG- | ✓ |
| Pascual | prior to | Nagel, | 533R | | | | CRC-7, | RAST | |
| et al, | colonos- | Germany | | | | | Ctrl-10, | | |
| 2015 | copy | | | | | | Total- | | |
| [27] | | | | | | | 28 | | |
| Flemer | Fecal | AllPrep, | F: | V3-V4 | Illumina- | F | CRA-80, | Author | ✓ |
| et al, | samples | Qiagen | GGNGGC | | MtSeq | | CRC-0, | | |
| 2016 | collected | | WGCAG | | | | Ctrl-43, | | |
| [28] | prior to | | R: | | | | Total- | | |
| | bowel | | GTCTCGT | | | | 37 | | |
| | prep, | | GGGCTCG | | | | | | |
| | biopsy | | | | | | | | |
| | samples | | | | | | | | |
| | obtained | | | | | | | | |
| | prior to | | | | | | | | |
| | resection | | | | | | | | |
| Sobhani | No anti- | G'NOME | V3F, | V3-V4 | 454-FLX | NA | CRA-0, | NA | X |
| et al, | biotic | DNA | V4R | | | | CRC-6, | | |
| 2011 | intake, | | | | | | Ctrl-6, | | |
| [15] | prior to | | | | | | Total- | | |
| | Colonos- | | | | | | 12 | | |
| | copy | | | | | | | | |
| Chen | No anti- | bead- | 27F, | V1-V3 | 454-FLX | NA | CRA-47, | NA | X |
| et al, | biotics, | beating | 533R | | | | CRC-0, | | |
| 2013 | adequate | and | | | | | Ctrl-47, | | |
| [32] | recovery | phenol- | | | | | Total- | | |
| | time | chloro- | | | | | 94 | | |
| | post col- | form | | | | | | | |
| | onoscopy | purifi- | | | | | | | |
| | | cation | | | | | | | |
| Ahn | Histori- | MoBio | 347F, | V3-V4 | 454-FLX | NA | CRA-0, | NA | X |
| et al, | cally | Powersoil | 803R | | | | CRC-47, | | |
| 2013 | stored | | | | | | Ctrl-94, | | |
| [31] | fecal | | | | | | Total- | | |
| | bio- | | | | | | 141 | | |
| | specimens | | | | | | | | |
| | from his- | | | | | | | | |
| | tologically | | | | | | | | |
| | confirmed | | | | | | | | |
| | colorectal | | | | | | | | |
| | adenoma and | | | | | | | | |
| | cancer | | | | | | | | |
| | cases and | | | | | | | | |
| | matched | | | | | | | | |
| | controls | | | | | | | | |
| | prior to | | | | | | | | |
| | initiation | | | | | | | | |
| | of | | | | | | | | |
| | treatment | | | | | | | | |
| Goedert | Partici- | EDTA- | 319F, | V3-V4 | Illumina- | NA | CRA-24, | NA | X |
| et al, | pants who | lysozyme- | 806R | | MtSeq | | CRC-2, | | |
| 2015 | presented | lauryl | | | | | Ctrl-20, | | |
| [30] | for CRC | sacrosyl | | | | | Total- | | |
| | screening, | extrac- | | | | | 46 | | |
| | prior to | tion and | | | | | | | |
| | CRC/ | cesium | | | | | | | |
| | adenoma | chloride- | | | | | | | |
| | colonos- | ethidium | | | | | | | |
| | copy or | bromide | | | | | | | |
| | treatment | purifi- | | | | | | | |
| | | cation | | | | | | | |

Abbreviations: Seq Plat: Sequencing Platform, Seq dir: Sequencing direction, F: Forward (5′-3′) direction, R: Reverse (3′-5′) direction, CRC: Colorectal Cancer, CRA: Colorectal adenoma, Ctrl: Control V1, V3, V4: Variable regions of the 16S rRNA gene
✓ indicate studies included in the analysis, X indicates studies for whom data was not available

TABLE 2B
Sequence statistics of studies included in the meta-analysis

| | | | Biospecimen | | |
|---|---|---|---|---|---|
| | | | processed | Biospecimen | Avg |
| | | | through | processed | Reads/biospecimen |
| | Raw seq | Avg read len | QIIME- | through SS- | reported in |
| Study acronym | counts | (±SD) | CR | UP | manuscript |
| Wang_V3_454 | 347716 | 186.4 ± 34.9 | 102 | 102 | 2734 ± 460 |
| Chen_V13_454 | 508160 | 444.2 ± 145.8 | 42 | 42 | 4253 |
| Wu_V3_454 | 1076196 | 180.4 ± 46.9 | 31 | 31 | 18522 |
| Weir_V4_454 | 199750 | 250.9 ± 99.6 | 13 | 13 | 1250 |
| Brim_V13_454 | 700890 | 416.6 ± 149.4 | 12 | 12 | NA |
| Zackular_V4_MiSeq | 11243169 | 252.9 ± 1.4 | 90 | 90 | median: 95464 |
| Zeller_V4_MiSeq | 4346191 | 254.5 ± 13.8 | 129 | 129 | NA |
| Pascual_V13_454 | 58850 | 326.2 ± 76.2 | 28 | 28 | 3,494 |
| Flemer_V34_MiSeq | 1567117 | 448.0 ± 10.3 | 80 | 80 | NA |

| | Fraction | | | |
|---|---|---|---|---|
| | of raw | | | |
| | reads | Fraction of | | |
| | assigned | raw reads | | |
| | to OTUs | assigned to | | |
| | (QIIME- | OTUs (SS- | Avg reads ± SD | Avg reads ± SD |
| Study acronym | CR) | UP) | QIIME-CR | SS-UP |
| Wang_V3_454 | 81.1% | 92.2% | 2763.7 ± 456.8 | 2811.5 ± 463.1 |
| Chen_V13_454 | 26.4% | 64.7% | 3190.5 ± 617.6 | 3756.7 ± 579.7 |
| Wu_V3_454 | 53.1% | 75.5% | 18430.2 ± 10572.5 | 17886.4 ± 10602.3 |
| Weir_V4_454 | 6.2% | 81.2% | 688.3 ± 1317.6 | 2641.7 ± 5142.7 |
| Brim_V13_454 | 66.5% | 81.3% | 38854.4 ± 7935.2 | 40362.17 ± 8006.3 |
| Zackular_V4_MiSeq | 81.9% | 96.2% | 109664.5 ± 56565.3 | 128029.4 ± 67747.5 |
| Zeller_V4_MiSeq | 85.4% | 81.7% | 287613.4 ± 159160.3 | 293229.5 ± 162297.9 |
| Pascual_V13_454 | 39.4% | 92.1% | 1008.7 ± 1058.3 | 2358 ± 52567.5 |
| Flemer_V34_MiSeq | 45.5% | 86.1% | 8909.7 ± 3204.1 | 16866.0 ± 5582.5 |

Abbreviations:
seq: sequence,
Avg: average,
SD: Standard Deviation,
QIIME-CR: QIIME closed reference OTU picking;
SS-UP: Strain Select, UPARSE bioinformatics pipeline.
Avg reads ± SD per sample is reported for each pipeline

Nine of these had sequence data in public repositories (e.g., the Sequence Read Archive (SRA), European Nucleotide Archive (ENA), MG-RAST) or provided raw data upon request. Eight of these had CRC or CRA and controls in their study design. [10-14, 26-28] One study evaluated fecal samples exclusively from CRA cases and controls. [29] Raw sequence data for the remaining four studies was not publicly available, was not provided upon request, or was available through controlled access only. [15, 30-32] Accordingly, these studies were not included in the analysis.

We compiled 16S rRNA gene sequencing data from the nine studies. Study sizes varied from 12 to 129 subjects, and we analyzed a total of 59,163,765 raw 16S rRNA gene sequences through two bioinformatics pipelines, QIIME-CR and SS-UP. This combined data set consisted of 195 CRC, 79 CRA, and 235 controls. Sequence lengths and counts were non-uniform across studies, but SS-UP retained a greater number of reads than QIIME-CR

Patient Metadata

Those participants for whom disease status (i.e., CRC, CRA, or control) was available were included in the analysis. Zeller et al [10] excluded large adenomas from their analysis and combined small adenomas as controls. We evaluated all of these samples as CRA specimens. The clinical variables of age, gender, BMT (or height and weight), and the outcome of fecal occult blood test (FORT) were also available for three studies. [10-12]

Bioinformatics Analysis

As noted above, each study was analyzed using two bioinformatics pipelines, an open-source closed-reference operational taxonomic unit (OTU) assignment pipeline implemented in QIIME (QIIME-CR) [33] and a pipeline which aligns fecal 16S sequences against references in the StrainSelect database (secondgenome.com/StrainSelect) and conducts de novo clustering using the UPARSE methodology (SS-UP). [34]

The rationale behind using two pipelines was to assess an alternate approach to closed-reference OTU picking, which is commonly used in microbiome meta-analyses, and determine how different OTU clustering methodologies might affect downstream performance of the composite biomarker for CRC. SS-UP had the added advantage of strain-level annotations for some OTUs, whereas QIIME-CR offered species-level resolution for some. We sought to determine if microbiome-based differences between diseased and control subjects were substantial enough to discriminate among subjects using either bioinformatics pipeline, or, if the differences were subtle, such that a specialized algorithm might be required. For each pipeline, quality filtering criteria and sequence utilization are provided in Tables 3A-3B and details regarding implementation of each pipeline are provided in the Supplementary Methods.

TABLE 3A
Length filtering criteria used to generate reads for OTU clustering.

Length Filtering (min-max)

| Study | QIIME-CR | SS-UP |
|---|---|---|
| Wang_V3_454 | 100-500 | 80-220 |
| Chen_V13_454 | 100-600 | 150-600 |
| Wu_V3_454 | 100-500 | 80-220 |
| Weir_V4_454 | 100-1000 | 80-300 |
| Brim_V13_454 | 100-600 | 150-600 |
| Zackular_V4_MiSeq | NA | NA |
| Zeller_V4_MiSeq | NA | NA |
| Pascual_V13_454 | 100-600 | 150-600 |
| Flemer_V34_MiSeq | NA | NA |

TABLE 3B
Median sequence length of reads utilized for each
pipeline. Reads were mapped to strain-level OTUs
and clustered into de novo OTUs in SS-UP, and they
were mapped to reference OTUs using QIIME-CR

| | SS-UP | | QIIME-CR |
|---|---|---|---|
| | Strain | de novo | Reference |
| Study | OTUs | OTUs | OTUs |
| Wang_V3_454 (Forward) | 156 | 142 | 142 |
| Wang_V3_454 (Reverse) | 156 | 142 | 157 |
| Chen_V13_454 | 487 | 485 | 486 |
| Wu_V3_454 (Forward) | 155 | 154 | 155 |
| Wu_V3_454 (Reverse) | 156 | 142 | 154 |
| Weir_V4_454 | 114 | 298 | 296 |
| Brim_V13_454 | 487 | 486 | 484 |
| Zackular_V4_MiSeq | 253 | 253 | 251 |
| Zeller_V4_MiSeq | 253 | 253 | 253 |
| Pascual_V13_454 | 402 | 445 | 297 |
| Flenter_V34_MiSeq | 441 | 440 | 441 |

Abbreviations: QIIME-CR: QIIME closed reference method; SS-UP: Strain Select, UPARSE method.

QIIME-CR Processing:

For the QIIME-CR pipeline, quality filtering and demultiplexing for the 454 datasets was done using the split_libraries.py command in QIIME 1.8 [10]. Minimum and maximum read lengths were chosen based on the target amplicon length to filter out truncated or erroneously long reads for both QIIME-CR and SS-UP. The filtering lengths used for each are summarized in Tables 3A-3B. Additionally, we used the default parameters for quality filtering (i.e., exclusion of sequences with >6 ambiguous bases, homopolymer runs >6 nucleotides, mismatches to the primer or barcode sequence). For Illumina data, we used the multiple_join_paired_ends.py and multiple_split_libraries_fastq.py scripts from QIIMF 1.9, as they could process multiple files simultaneously. The quality filtering parameters were set to default (i.e. reads were truncated at the first instance of a low-quality base call (q<20) and reads were excluded if <75% of the length of the original read). QIIME 1.9.0 was used only for initial fastq processing for the large MiSeq-based studies. OTU clustering and taxonomy assignment for all studies was performed using QIIME 1.8.0.

Quality-filtered and demultiplexed datasets from both the 454 and Illumina studies were assigned to reference based OTUs using pick_closed_reference_otus.py, which employed uclust 1.2.22q [11] with reverse strand matching enabled. In this strategy, input sequences were aligned to a pre-defined cluster centroid in the reference database (Greengenes_13_8). [12] A sequence was retained only if it matched the reference dataset at a threshold of 97% identity. A disadvantage of this approach is the disregard of reads that are dissimilar to a reference. For one study [14], fasta-formatted sequence files were shared on the MG-RAST repository, but qual files were omitted. Hence quality filtering was not possible or this study and only length trimming was done prior to clustering for both the QIIME-CR and SS-UP pipelines. In two studies, [27, 13] 454 was used to collect both F and R reads but since they were not paired, reads were assessed as the sum of two libraries of single ended reads.

SS-UP Processing:

Strain Select UPARSE (SS-UP) (Second Genome, Inc) pipeline utilized the StrainSelect database, a collection of high-quality sequence and annotation data derived from bacterial and archaeal strains that can be obtained from an extant culture collection (secondgenome.com/StrainSelect) (publication in preparation), and conducts de novo clustering of all sequences without strain hits using the UPARSE methodology (SS-UP). For SS-UP, Illumina paired-end sequenced reads were merged using USEARCH fastq_mergepairs with default settings except for dataset-specific cutoffs for fastq_minmergelen and fastq_maxmergelen (Tables 3A-3B). All resulting merged sequences were compared against StrainSelect v2014-02-20 using USEARCH's usearch_global. 454 single-end reads were first quality trimmed from the N-terminal end using PrinSeq-lite [26] and parameters ‘-trim_ns_left 1-trim_ns_right 1-min_len $MIN_LEN-trim_qual_right 20’ (minimal length values per dataset are summarized in Tables 3A-3B) before comparison to StrainSelect using USEARCH's usearch_global. Distinct strain matches were defined as those with ≥99% identity to a 16S sequence from the closest matching strain and a lesser identity (even by one base) to the second closest matching strain, Those distinct hits were summed per strain and a strain-level OTU abundance table was created. The remaining sequences were filtered by overall read quality using USEARCH's fastq_maxee and a MAX_EE value of 1, length-trimmed to the lower boundary of the 95% interval of the read length distribution (for datasets with an uneven read length distribution length-trimming to the shortest read length is strongly affected by very short reads; the 95% interval is used to compensate for this outlier effect), de-replicated, sorted descending by size and clustered at 97% identity with USEARCH (fastq_filter, derep_fulllength, sortbysize, cluster_otus), USEARCH cluster_otus discards likely chimeras. A representative consensus sequence per de novo OTU was. For each study, de novo OTUs with abundance of less than 3 in a study were discarded as spurious. All sequences that went into the comparison against StrainSelect but did not end up in a strain OTU were then mapped to the set of representative consensus sequences (≥97% identity) to generate a de novo OTU abundance table. Representative strain-level OTU sequences and representative de novo OTU sequences were assigned a Greengenes [12] taxonomic classification via mothur's bayesian classifier [28] at 80% confidence; the classifier was trained against the Greengenes reference database (version 13_5) of 16S rRNA gene sequences. Both Greengenes version 13_5 used for SS-UP and version 13_8 used for QIIME-CR contain the same set of reference sequences. In the 13_8 version, additional taxonomic terms were manually curated, but the reference OTUs and phylogenetic trees remained unchanged. Where standard taxonomic names have not been established, a hierarchical taxon identifier was used (for example “97otu15279”). Strain-level OTU abundances and taxonomy-mapped de novo OTU abundances from all studies were merged and used for further analysis. The SS-UP approach allowed all high-quality sequences to be counted, and the taxonomic classification of the de novo OTUs permitted de novo OTUs with conserved taxonomy to be compared across studies.

Samples with <100 sequences after quality filtering and OUT assignment for either bioinformatics pipeline were excluded from both all further analysis. In all cases, any sample that had <100 sequences in one pipeline had <100 sequences in the other.

Statistical Analysis

The R package phyloseq was used for determining global community properties such as alpha diversity, beta diversity metrics such as the Bray-Curtis and Jaccard index, principle coordinate scaling of Bray-Curtis dissimilarities, Firmicutes/Bacteroidetes (F/B) ratio and differential abundance analysis. Two-sample permutation t-tests using Monte-Carlo resampling were used to compare the alpha diversity estimates and F/B ratio across CRC and controls and CRA and controls. Permutational analysis of variance (PERMANOVA) was used to test whether within group distances were significantly different from between group distances using the adonis function in the vegan package. Multivariate homogeneity of group dispersions was tested with vegan using the betadisper function. Differential abundance of QIIME OTUs and SS-UP OTUs across CRC cases and controls was evaluated adjusting for Study as a confounding factor in the DESeq2 design (˜Study+disease status). OTUs were considered significantly different if their False Discovery Rate (FDR) adjusted Benjamin Hochberg (BH) p value was <0.1 and estimated log 2-fold change was >1.5 or <−1.5.

The Random Effects model (REM) considered the eight studies with CRC-control samples as a sample of a larger number of studies and inferred the likely outcome if a new study, were performed. The CRC-fecal microbiome studies were dissimilar in terms of their methods as well as patient demographics. These differences may introduce heterogeneity among true effects. The RE model treats this heterogeneity as random. Specifically, in addition to the pooled analysis mentioned above we estimated study by study DESeq2 log 2 fold changes as effect size estimates and the standard error associated with them as corresponding sampling variances as an input for the REM. OTUs that occurred as differentially abundant by DESeq2 in at least 5 studies (i.e 5 or 6 or 7 or 8 studies) for the CRC vs control comparison and either 3 or 4 studies for the CRA vs control comparison were retained for the analysis. The resulting RE model p-values were FDR corrected for multiple comparisons across taxa OTUs and forest plots were plotted for significant OTUs. We also plotted relative abundances of these OTUs across several studies to estimate how the log fold changes in cases as compared to controls reflected in the prevalence of the actual OTUs.

To determine the predictive power of microbial taxa for the random forest classifier, the number of predictor features randomly sampled for splitting at each node in the decision tree commonly known as miry was tuned as (0.5, 1, 1.5, 1.75, 2.5, 3.0)*(square root of total number of microbial predictors). Models were internally cross-validated ten-fold times with five repeats to avoid over-fitting, Tuning area under receiver operating characteristic (AUROC) curve with the largest value was used to select the optimal model. RE models to predict disease outcome were built for clinical markers only (for studies where clinical metadata was available (n=3 studies, 156 samples)), microbial markers only (for all samples and studies (n=8 studies, 344 samples) as well as the subset of samples for which complete clinical metadata was available n=3 studies, 156 samples)), and a combination of both clinical and microbial markers (n=3 studies, 156 samples). Continuous variables among the clinical metadata such as age and BMI were centered and scaled prior to building the RE models. To estimate if any particular study disproportionately affected the optimal AUROC value of the classifier, we conducted a leave one study out analysis and estimated the classifier accuracy after each study was omitted. We also determined classifiers for individual studies to compare how the composite classifier fared with homogenously processed features from individual studies. Recursive feature elimination using 10 fold cross-validation with five repeats was used to identify the most informative microbial taxa for classification using the de function. To determine the generalizability of the composite microbial biomarker, the leave one study out cohort (test set) classifier was used to predict the disease outcome in the study that was left out (validation set) using the predict.train function. ROC's were plotted for the above models using the pROC package. [29] Differences in the AUROC were tested statistically with DeLong's test within the package.

Resulting OTU tables from each pipeline were analyzed using univariate and multivariable techniques, and all statistical analysis was conducted in R (version 3.2.1). Samples from patients documented as receiving chemotherapy or radiotherapy, having <100 reads per sample, and OTUs occurring in <5% of all samples were excluded from analysis for both pipelines. Data were rarefied for alpha diversity comparisons to a depth of 1000 without replacement but were not rarefied for any other analyses. [35] Global community properties were evaluated using phyloseq [35, 36] and permutational analysis of variance (PERMANOVA) was performed with the adonis function in vegan. [37] Differential abundance analysis (between cases and controls) was performed using DESeq2 at the species (QIIMF-CR) and strain (SS-UP) levels. To identify microbial features that occurred universally in CRC and CRA cases and were robust to technical variation, we applied a random effects model (REM) to obtain adjusted log 2fold change summary estimates (considered significant at FDR p<0.1). This was performed using the metafor package in R and treating study as a random effect. [38] Random Forest (RF) models were used to determine whether a composite fecal microbial biomarker could discriminate CRC and CRA cases versus controls. Combined relative abundance-transformed OTU counts across all studies were analyzed using the caret package in R. [39, 40] Additional details regarding the analysis are provided in the Supplementary Methods.

Results

Bray-Curtis dissimilarity and the Jaccard index were used to evaluate the effects of abundance and carriage, respectively. Ordination analysis revealed substantial variation among samples with respect to microbial community composition and showed that ordinations from SS-UP captured a greater amount of the total variation along the first two axes than did those from QIIME-CR. Separation along axis 1 occurred primarily by study, followed by variable region and sequencing platform. Given the large differences on those parameters, separation between cases and controls was not readily observed.

PERMANOVA indicated that microbiome composition differed significantly as a function of disease status, however the lack of homogeneity of variance between cases and controls is likely to have influenced this result. After confirming homogeneity of variance, microbiome composition was significantly different by PERMANOVA across BMI categories, sequencing platforms FOBT test results, and metastatic disease classification (denoted by M in TNM staging) (where information available) for either informatics pipeline or sometimes both. (Table 4).

TABLE 4
Comparison of microbiome composition groups across clinical, demographic and
technical variables using PERMANOVA.

| | SS- | | | | | |
|---|---|---|---|---|---|---|
| | UP p- | | QIIME-CR | | | |
| Variable | value | Betadisper p | p-value | Betadisper p | Classes | Sample Count^{#} |
| Disease | 0.001 | 0.0006381 | 0.001 | 1.9 * 10⁻⁹ | adenoma, | 79, 195, 235 |
| Status | | | | | carcinoma, control | |
| BMI | 0.001 | 0.3218 | 0.002 | 0.5618 | I, II, III | 128, 123, 66 |
| category | | | | | | |
| Target | 0.001 | 2.45 * 10⁻¹⁶ | 0.001 | 5.9 * 10⁻¹⁴ | V1_V3, V1_V4, V3, | 35, 42, 133, 67, |
| Gene | | | | | V3_V4, V4 | 232 |
| platform | 0.001 | 2.3 * 10⁻⁸ | 0.001 | 0.4705 | 454_FLX, | 169, 54, 286 |
| | | | | | 454_Titanium, | |
| | | | | | MiSeq | |
| Study | 0.001 | 8.8 * 10⁻⁹ | 0.001 | 2.2 * 10⁻¹⁶ | Brim_V13_454, | 12, 42, 67, 23, |
| | | | | | Chen_V13_454, | 102, 13, 31, 90, |
| | | | | | Flemer_V34_MiSeq, | 129 |
| | | | | | Pascual_V13_454, | |
| | | | | | Wang_V3_454, | |
| | | | | | Weir_V4_454, | |
| | | | | | WuZhu_V3_454, | |
| | | | | | Zack_V4_MiSeq, | |
| | | | | | Zeller_V4_MiSeq | |
| Sex | 0.022 | 5.15 * 10⁻⁵ | 0.063 | 0.01072 | F, M | 134, 214 |
| Age | 0.001 | 0.00262 | 0.001 | 0.1039 | ≤40, 41-55, 56-70, | 14, 162, 191, 87 |
| categories | | | | | >70 | |
| FOBT | 0.001 | 0.1026 | 0.003 | 0.01206 | N, P | 178, 53 |
| T* | 0.747 | | 0.469 | | T1, T2, T3, T4, Tis | 13, 38, 20.1 |
| N* | 0.076 | | 0.001 | | N0, N1, N1a, N1b, | 34, 32, 4, 3, 1, 2, |
| | | | | | N2, N2a, N2b, NX | 2, 1 |
| M* | 0.006 | 0.114 | 0.001 | 6.7 * 10⁻⁵ | M₀, M₁ | 59, 20 |
| Nationality | 0.001 | 8.7 * 10⁻¹² | 0.001 | 6.5 * 10⁻¹³ | Chinese, French, | 172, 129, 67, |
| | | | | | Irish, Spanish, | 23, 118 |
| | | | | | United_States | |
| Region | 0.001 | 1.24 * 10⁻¹¹ | 0.001 | 0.4031 | Asian, European, | 172, 219, 118 |
| | | | | | North_American | |

Abbreviations:
PERMANOVA: Permutational ANOVA,
SS-UP: Strain Select UPARSE,
QIIME-CR: QIIME closed reference
OTU picking,
BMI: Body Mass Index,
V1-V4: Variable regions 1 through 4 in the 16S rRNA gene,
FOBT: Fecal Occult Blood test
#: Sample count is in the order in which they occur in the ‘Classes’ column
*TNM: TNM is a cancer staging system where T stands for the size of the original tumor (T1-T4 ranging from smallest to largest respectively, Tis: carcinoma in situ),
N stands for lymph node involvement (N0 to N2 denoting less to high lymph node infiltration, Nx: lymph node involvement cannot be evaluated) and M denotes whether the cancer has metastasized to different parts of the body (M0: not metastasized, M1: Metastasized)

Global community properties measured by alpha diversity indices were similar between CRC cases and controls in SS-UP and CRA cases and controls in both the SS-UP and QIIMF-CR pipelines. The Shannon and inverse Simpson indices were significantly lesser in CRA cases relative to controls in the QIIMF-CR pipeline by Monte-Carlo permutation-based t-tests. (Table 5) The Firmicutes/Bacteroidetes ratio did not differ in either CRC or CRA cases relative to controls.

TABLE 5
Alpha diversity distribution in samples with different
disease states across both pipelines

| | Mean (SD) | p-value | Median |
|---|---|---|---|
| QIIME-CR | Shannon, | | Shannon |
| Control | 4.1(0.7) | 0.012 | 4.1 |
| CRC | 3.9(0.8) | | 3.9 |
| | InvSimpson, | | InvSimpson. |
| Control | 29.8 (22.9) | 0.05 | 23.1 |
| CRC | 25.5(20.4) | | 19.2 |
| QIIME-CR | Shannon | | Shannon |
| Control | 4.0 (0.9) | 0.6 | 4.2 |
| CRA | 4.1(0.7) | | 4.3 |
| | InvSimpson. | | InvSimpson. |
| Control | 25.9(17.7) | 0.8 | 20.5 |
| CRA | 25.0(13.1) | | 25.8 |
| SS-UP | Shannon | | Shannon |
| Control | 3.2 (0.6) | 0.4 | 3.2 |
| CRC | 3.1(0.6) | | 3.2 |
| | InvSimpson. | | InvSimpson. |
| Control | 14.6 (8.8) | 0.3 | 12.8 |
| CRC | 13.8 (7.9) | | 12.1 |
| SS-UP | Shannon | | Shannon |
| Control | 4.1 (0.7) | 0.7 | 4.1 |
| CRA | 3.9 (0.8) | | 3.9 |
| | InvSimpson. | | InvSimpson. |
| Control | 29.8 (22.9) | 0.5 | 23.1 |
| CRA | 25.5 (20.4) | | 19.2 |

Abbreviations: QIIME-CR, QIIME closed reference OTU picking, SD. Standard Deviation, CRC—Colorectal Cancer, CRA—Colorectal Adenoma, SS-UP: Strain Select - UPARSE, p-value: p-value for difference in mean across disease categories determined by t-test with Monte Carlo permutations.

Post-filtering, a total of 895 and 3511 OTUs were retained for the SS-UP and QIIME-CR pipelines, respectively, for the analysis of differential abundances between CRC cases and controls. Peptostreptococcus anerobius, Parvimonas, Porphyromonas, Akkermansia muciniphila, and Fusobacterium sp. were significantly enriched in CRC cases relative to controls across both pipelines. (Table 6)

TABLE 6
Differential abundance in CRC cases as compared to controls using SS-UP

| | base | log2 | | | | | |
|---|---|---|---|---|---|---|---|
| OTU | Mean | FC | lfc SE | stat | p | padj | Taxonomy |
| OTU1167 | 5.60 | 2.36 | 0.49 | 4.84 | 1.27E−06 | 5.76E−05 | Firmicutes; Parvimonas; 97otu 12932; 72331 |
| OTU1169 | 1.24 | 4.17 | 0.52 | 8.00 | 1.28E−15 | 2.0E−13 | Firmicutes; Parvimonas; 97otu 12932; unclassified |
| OTU1172 | 0.91 | 1.65 | 0.51 | 3.25 | 1.17E−03 | 1.64E−02 | Firmicutes; Parvimonas; unclassified; unclassified |
| OTU1345 | 8.29 | 1.88 | 0.39 | 4.86 | 1.17E−06 | 5.71E−05 | Firmicutes; 94otu24753; 97otu29453; unclassified |
| OTU1407 | 0.51 | 1.64 | 0.42 | 3.93 | 8.66E−05 | 1.96E−03 | Firmicutes; unclassified; unclassified; unclassified |
| OTU1622 | 1.03 | 1.76 | 0.60 | 2.93 | 3.35E−03 | 3.72E−02 | Firmicutes; 94otu 1007; unclassified; 19335 |
| OTU1750 | 10.33 | 2.17 | 0.41 | 5.34 | 9.47E−08 | 6.66E−06 | Firmicutes; 94otu41 928; 97otu5583; unclassified |
| OTU1978 | 12.49 | 2.88 | 0.40 | 7.16 | 8.26E−13 | 1.05E−10 | Firmicutes; unclassified; unclassified; 48865 |
| OTU1998 | 24.44 | 2.16 | 0.37 | 5.80 | 6.54E−09 | 5.17E−07 | Firmicutes; unclassified; unclassified; 89342 |
| OTU2045 | 11.07 | 2.52 | 0.53 | 4.76 | 1.96E−06 | 7.30E−05 | Firmicutes; Peptostreptococcus; 97otu2093; 84165 |
| OTU2049 | 1.59 | 4.51 | 0.51 | 8.77 | 1.79E−18 | 3.77E−16 | Firmicutes; Peptostreptococcus; anaerobius; |
| | | | | | | | unclassified |
| OTU2095 | 0.82 | 2.36 | 0.55 | 4.27 | 1.92E−05 | 5.51E−04 | Firmicutes; 94otu 13618; 97otu 15286; unclassified |
| OTU2389 | 9.96 | 2.41 | 0.48 | 5.03 | 4.91E−07 | 2.82E−05 | Firmicutes; Anacrotruncus; 97otu35713; unclassified |
| OTU2502 | 4.62 | 2.07 | 0.64 | 3.24 | 1.19E−03 | 1.64E−02 | Firmicutes; Ruminococcus; 97otu83887; unclassified |
| OTU2573 | 1.51 | 2.98 | 0.62 | 4.79 | 1.64E−06 | 6.91E−05 | Firmicutes; Dialister; 97otu23808; 82849 |
| OTU2589 | 11.26 | −1.62 | 0.42 | −3.91 | 9.21E−05 | 2.01E−03 | Firmicutes; Dialister; unclassified; unclassified |
| OTU2703 | 1.96 | −1.57 | 0.48 | −3.29 | 1.01E−03 | 1.55E−02 | Firmicutes; 94otu36460; 97otu 6478; 61378 |
| OTU2724 | 1.05 | 1.70 | 0.45 | 3.75 | 1.75E−04 | 3.35E−03 | Firmicutes; Bulleidia; moorei; unclassified |
| OTU2773 | 2.02 | 1.76 | 0.50 | 3.49 | 4.86E−04 | 8.80E−03 | Fusobacteria; Fusobacterium; 97out44835; unclassified |
| OTU2790 | 5.36 | 1.93 | 0.38 | 5.06 | 4.19E−07 | 2.65E−05 | Fusobacteria; Fusobacterium; unclassified; unclassified |
| OTU295 | 1.06 | 2.03 | 0.48 | 4.22 | 2.47E−05 | 6.81E−04 | Actinobacteria; unclassified; unclassified; unclassified |
| OTU3042 | 0.97 | 2.22 | 0.50 | 4.44 | 8.88E−06 | 2.91E−04 | Proteobacteria; Succinivibrio; unclassified; unclassified |
| OTU3069 | 442.41 | 1.65 | 0.36 | 4.61 | 4.05E−06 | 1.42E−04 | Proteobacteria; 94otu9652; 97otu 2810; unclassified |
| OTU3116 | 1.10 | 1.57 | 0.39 | 3.99 | 6.59E−05 | 1.61E−03 | Proteobacteria; unclassified; unclassified; 26180 |
| OTU3191 | 146.79 | 2.98 | 0.30 | 9.82 | 9.47E−23 | 5.99E−20 | Proteobacteria; unclassified; unclassified; unclassified |
| OTU3364 | 146.86 | 1.52 | 0.35 | 4.37 | 1.2A4E−05 | 3.75E−04 | Verrucomicrobia; Akkermansia; |
| | | | | | | | muciniphila; unclassified |
| OTU567 | 0.77 | 3.45 | 0.57 | 6.08 | 1.17E−09 | 1.23E−07 | Bacteroidetes; Porphyromonas; 97otu52506; 84846 |
| OTU569 | 8.32 | 5.10 | 0.56 | 9.04 | 1.63E−19 | 5.16E−17 | Bacteroidetes; Porphyromonas; 97otu52506; |
| | | | | | | | unclassified |
| OTU624 | 31.45 | 2.11 | 0.55 | 3.82 | 1.35E−04 | 2.75E−03 | Bacteroidetes; Prevotella; 97otu94784; unclassified |
| OTU910 | 15.80 | 1.91 | 0.38 | 4.99 | 5.92E−07 | 3.12E−05 | Firmicutes; Enterococcus; unclassified; unclassified |
| OTU954 | 2.65 | −2.09 | 0.52 | −4.03 | 5.69E−05 | 1.44E−03 | Firmicutes; Lactobacillus; ruminis; unclassified |
| OTU969 | 4.60 | 2.01 | 0.48 | 4.20 | 2.67E−05 | 7.03E−04 | Firmicutes; Lactobacillus; unclassified; unclassified |

Abbreviations:
CRC: Colorectal cancer,
SS-UP: Strain Select-UPARSE,
OTU: Operational Taxonomic Unit,
LogFC: Log2Fold Change,
lfcse: Log2Fold Change standard error,
stat: Wald test statistic,
p: p-value associated with Wald test,
padj: FDR adjusted p-value
Base Mean: average of the normalized count values, dividing by size factors Positive Log2Fold Change indicates enriched in CRC fecal samples as compared to controls and negative value indicates enriched in control samples as compared to CRC.
“97otu12932” describes a 97% (species-level) OTU cluster for which no standard taxonomic name has been assigned.
Taxonomy notation: phylum; genus; species; strain. For numeric strain annotations please refer to www.secondgenome.com/solutions/resources/data-analysis-tools/strainselect/ Positive Log2Fold Change indicates enriched in CRC fecal samples as compared to controls and negative value indicates enriched in control samples as compared to CRC

The SS-UP pipeline identified significant enrichment of specific strains in CRC cases, including Porphyromonas asaccharolytica ATCC 25260 and Parvimonas micra ATCC 33270. Significant enrichment of Pantoea agglomerans in CRC cases was also identified from QIIME-CR (Table 7).

TABLE 7
Differential abundance in CRC cases as compared to controls (QIIME-CR)

| | Base | log2 | | | | | |
|---|---|---|---|---|---|---|---|
| OTU | Mean | FC | IfcSE | stat | pvalue | padj | Taxonomy |
| OTU1105984 | 15.67 | −1.97 | 0.47 | −4.21 | 2.53E−05 | 2.64E−03 | Bacteroidetes; Bacteroidaceae; Bacteroides; |
| OTU114462 | 1.83 | 1.66 | 0.52 | 3.16 | 1.56E−03 | 4.01E−02 | Proteobacteria; Enterobacteriaceae; unc; |
| OTU114510 | 3.82 | 1.79 | 0.34 | 5.25 | 1.53E−07 | 1.10E−04 | Proteobacteria; Enterobacteriaceae; Escherichia; coli |
| OTU122049 | 1.22 | 1.69 | 0.52 | 3.22 | 1.27E−03 | 3.44E−02 | Proteobacteria; Enterobacteriaceae; unc; |
| OTU13986 | 1.87 | 2.01 | 0.45 | 4.44 | 9.00E−06 | 1.10E−03 | Firmicutes; Lachnospiraceae; unc; |
| OTU192963 | 15.00 | 1.71 | 0.41 | 4.20 | 2.70E−05 | 2.69E−03 | Verrucomicrobia; Verrucomicrobiaceae; Akkermansia; |
| | | | | | | | muciniphila |
| OTU2119418 | 38.42 | 2.10 | 0.46 | 4.56 | 5.18E−06 | 7.11E−04 | Proteobacteria; Enterobacteriaceae; Pantoea; agglom |
| OTU2438396 | 0.93 | 2.07 | 0.60 | 3.44 | 5.78E−04 | 2.00E−02 | Fusobacteria; Fusobacteriaceae; Fusobacterium; |
| OTU2730944 | 1.00 | −1.83 | 0.55 | −3.31 | 9.18E−04 | 2.79E−02 | Bacteroidetes; Bacteroidaceae; Bacteroides; |
| | | | | | | | coprophilus |
| OTU2986828 | 7.58 | 1.69 | 0.42 | 4.02 | 5.78E−05 | 4.23E−03 | Firmicutes; Lachnospiraceae; unc; |
| OTU299267 | 2.05 | 1.60 | 0.45 | 3.52 | 4.33E−04 | 1.71E−02 | Proteobacteria; Enterobacteriaceae; unc; |
| OTU315223 | 14.64 | 1.91 | 0.48 | 3.94 | 8.26E−05 | 5.33E−03 | Firmicutes; Ruminococcaceae; Anaerotruncus; |
| OTU3562626 | 4.87 | 2.47 | 0.51 | 4.82 | 1.47E−06 | 4.03E−04 | Bacteroidetes; Bacteroidaceae; Bacteroides; |
| OTU358939 | 0.33 | 1.67 | 0.48 | 3.46 | 5.41E−04 | 1.95E−02 | Firmicutes; Lachnospiraceae; unc; |
| OTU360890 | 9.91 | 1.86 | 0.41 | 4.48 | 7.42E−06 | 9.58E−04 | Firmicutes;; unc; |
| OTU3799784 | 3.31 | 1.50 | 0.38 | 3.91 | 9.29E−05 | 5.83E−03 | Proteobacteria; Enterobacteriaceae; unc; |
| OTU3851391 | 36.89 | 1.67 | 0.45 | 3.70 | 2.13E−04 | 1.07E−02 | Firm icutes; Lachnospiraceae; Blautia; |
| OTU4318284 | 2.90 | 2.58 | 0.56 | 4.60 | 4.26E−06 | 6.23E−04 | Firmicutes; Veillonellaceae; Dialister; |
| OTU4333897 | 8.20 | 1.52 | 0.35 | 4.31 | 1.65E−05 | 1.91E−03 | Proteobacteria; Enterobacteriaceae; unc; |
| OTU4370024 | 0.86 | 1.68 | 0.47 | 3.58 | 3.39E−04 | 1.52E−02 | Firmicutes; Lachnospiraceae; unc; |
| OTU4377418 | 0.96 | 2.44 | 0.47 | 5.20 | 2.00E−07 | 1.10E−04 | Firmicutes; [Tissierellaceae]; Parvimonas; |
| OTU4378683 | 12.52 | 1.91 | 0.40 | 4.76 | 1.95E−06 | 4.75E−04 | Firmicutes; Lachnospiraceae; unc; |
| OTU4391262 | 20.25 | 1.62 | 0.33 | 4.93 | 8.38E−07 | 2.77E−04 | Proteobacteria; Enterobacteriaceae; unc; |
| OTU4393532 | 2.65 | 1.68 | 0.36 | 4.66 | 3.23E−06 | 5.78E−04 | Actinobacteria; Coriobacteriaceae; Eggerthella; lenta |
| OTU4416025 | 3.03 | 1.77 | 0.46 | 3.89 | 9.98E−05 | 6.08E−03 | Firmicutes; Lachnospiraceae; [Ruminococcus]; gnavus |
| OTU4425571 | 38.50 | 1.57 | 0.34 | 4.62 | 3.89E−06 | 6.09E−04 | Proteobacteria; Enterobacteriaceae; unc; |
| OTU4429981 | 10.02 | −2.48 | 0.53 | −4.64 | 3.43E−06 | 5.78E−04 | Firmicutes;; unc; |
| OTU4433823 | 5.97 | 1.62 | 0.40 | 4.06 | 4.86E−05 | 3.81E−03 | Bacteroidetes; Bacteroidaceae; Bacteroides; fragilis |
| OTU4442899 | 26.28 | 1.63 | 0.48 | 3.40 | 6.63E−04 | 2.14E−02 | Firmicutes;; unc; |
| OTU4446669 | 2.59 | 2.18 | 0.44 | 4.92 | 8.55E−07 | 2.77E−04 | Firmicutes; Ruminococcaceae; unc; |
| OTU4455308 | 1.39 | 1.92 | 0.48 | 3.99 | 6.51E−05 | 4.45E−03 | Firmicutes; Lachnospiraceae; unc; |
| OTU4457268 | 1.92 | 1.73 | 0.42 | 4.12 | 3.76E−05 | 3.17E−03 | Proteobacteria; Enterobacteriaceae; unc; |
| OTU4473664 | 0.67 | 2.16 | 0.61 | 3.56 | 3.74E−04 | 1.59E−02 | Firmicutes; Peptostreptococcaceae; Peptostreptococcus; |
| | | | | | | | anaerobius |
| OTU4475469 | 0.65 | 1.87 | 0.47 | 4.00 | 6.29E−05 | 4.45E−03 | Firmicutes; Erysipelotrichaceae; |
| | | | | | | | [Eubacterium]; dolichum |
| OTU4476950 | 0.38 | 1.94 | 0.51 | 3.82 | 1.31E−04 | 7.38E−03 | Firmicutes; [Tissierellaceae]; Anaerococcus; |
| OTU495451 | 16.65 | 4.30 | 0.48 | 9.02 | 1.87E−19 | 4.11E−16 | Bacteroidetes; Porphyromonadaceae; Porphyromonas; |
| OTU656881 | 12.20 | 1.63 | 0.35 | 4.67 | 3.07E−06 | 5.78E−04 | Proteobacteria; Enterobacteriaceae; Escherichia; coli |
| OTU782953 | 457.94 | 1.60 | 0.33 | 4.92 | 8.85E−07 | 2.77E−04 | Proteobacteria; Enterobacteriaceae; unc; |
| OTU816702 | 3.40 | 2.70 | 0.51 | 5.28 | 1.30E−07 | 1.10E−04 | Proteobacteria; Enterobacteriaceae; unc; |
| OTU828676 | 0.40 | 1.70 | 0.53 | 3.19 | 1.43E−03 | 3.72E−02 | Fusobacteria; Fusobacteriaceae; Fusobacterium; |
| OTU851704 | 11.48 | 1.93 | 0.45 | 4.24 | 2.19E−05 | 2.40E−03 | Firmicutes; [Tissierellaceae]; Parvimonas; |
| OTU851938 | 1.56 | 1.70 | 0.43 | 3.99 | 6.69E−05 | 4.45E−03 | Firmicutes; Erysipelotrichaceae; Bulleidia; moorei |
| OTU91557 | 2.34 | 1.90 | 0.51 | 3.70 | 2.14E−04 | 1.07E−02 | Proteobacteria; Enterobacteriaceae; unc; |

Abbreviations:
OTU: Operational Taxonomic Unit,
LogFC: Log₂Fold Change,
lfcse: Log₂Fold Change standard error,
stat: Wald test statistic,
pval: p-value associated with Wald test,
padj: FDR adjusted p-value,
unc: unclassified.
Base Mean: average of the normalized count values, dividing by size factors.
Positive Log2Fold Change indicates enriched in CRC fecal samples as compared to controls and negative value indicates enriched in control samples as compared to CRC.

In the CRA versus control comparison, 710 and 2586 OTUs were analyzed from the SS-UP and QIIME-CR pipelines, respectively. OTUs within the genera Prevotella, Methanosphaera, and Succinovibrio and species Haemophilus parainfluenzae were significantly enriched in both pipelines. SS-UP identified unique strains such as Synergistes family DSM 25858, Methanasphaera stadtmanae DSM 3091 as significantly differential abundant by DESeq. Akkermansia muciniphila was less abundant in CRA cases relative to controls by the QIIME-CR (Tables 8 and 9).

TABLE 8
Differential abundance in CRA cases as compared to controls (SS-UP)
Positive Log2Fold Change indicates enriched in CRA fecal samples as
compared to controls and negative value indicates enriched in control
samples as compared to CRA

| | Base | | | | | | |
|---|---|---|---|---|---|---|---|
| OTU | Mean | log2FC | lfcS E | stat | pvalue | padj | Taxonomy |
| OTU1004 | 88.37 | −2.72 | 0.55 | −4.94 | 7.93E−07 | 1.01E−04 | Firmicutes; Lactococcus; 97otu27091; unclassified |
| OTU1145 | 8.41 | −1.96 | 0.46 | −4.25 | 2.18E−05 | 1.66E−03 | Firmicutes; unclassified; unclassified; unclassified |
| OTU1223 | 7.55 | −4.45 | 0.79 | −5.67 | 1.42E−08 | 3.62E−06 | Firmicutes; 94otu2512; 97otu2859; unclassified |
| OTU1610 | 1135.54 | −1.77 | 0.48 | −3.71 | 2.06E−04 | 1.05E−02 | Firmicutes; [Ruminococcus]; 97otu99006; unclassified |
| OTU1649 | 3.33 | 2.72 | 0.62 | 4.39 | 1.11E−05 | 1.21E−03 | Firmicutes; 94otu13321; 97otu22055; unclassified |
| OTU1682 | 6.51 | −2.18 | 0.66 | −3.32 | 9.01E−04 | 2.99E−02 | Firmicutes; 94otu 18960; unclassified; unclassified |
| OTU1699 | 0.96 | 2.31 | 0.69 | 3.35 | 8.06E−04 | 2.93E−02 | Firmicutes; 94otu21297; 97otu23365; unclassified |
| OTU1825 | 89.38 | −2.90 | 0.58 | −5.03 | 4.92E−07 | 7.52E−05 | Firmicutes; Blautia; 97otu84279; unclassified |
| OTU2087 | 3.39 | 1.85 | 0.57 | 3.22 | 1.27E−03 | 3.25E−02 | Firmicutes; 94otu 12622; 97otu64265; unclassified |
| OTU214 | 0.69 | −2.35 | 0.73 | −3.23 | 1.22E−03 | 3.25E−02 | Actinobacteria; 94otu 15175; 97otu 16848; unclassified |
| OTU2337 | 123.98 | 1.80 | 0.49 | 3.69 | 2.21E−04 | 1.05E−02 | Firmicutes; 94otu5555; unclassified; unclassified |
| OTU2460 | 2.54 | −2.88 | 0.66 | −4.37 | 1.26E−05 | 1.21E−03 | Firmicutes; Ruminococcus; 97otu20971; unclassified |
| OTU2510 | 2242.93 | −2.06 | 0.51 | −4.05 | 5.20E−05 | 3.06E−03 | Firmicutes; Ruminococcus; bromii; 23783 |
| OTU2514 | 3.52 | 4.32 | 1.34 | 3.22 | 1.28E−03 | 3.25E−02 | Firmicutes; Ruminococcus; flavefaciens; unclassified |
| OTU2610 | 20.28 | −5.10 | 0.72 | −7.07 | 1.53E−12 | 1.17E−09 | Firmicutes; Megasphaera; 97otu8385; 33536 |
| OTU2681 | 6.39 | 2.83 | 0.85 | 3.31 | 9.38E−04 | 2.99E−02 | Firmicutes; [Eubacterium]; 97otu61417; 37647 |
| OTU3009 | 4.06 | 2.38 | 0.72 | 3.28 | 1.03E−03 | 3.01E−02 | Proteobacteria; Desulfovibrio; 97otu8883; unclassified |
| OTU3100 | 25.65 | 2.58 | 0.70 | 3.66 | 2.51E−04 | 1.13E−02 | Proteobacteria; Serratia; unclassified; unclassified |
| OTU3191 | 562.33 | 2.96 | 0.53 | 5.60 | 2.18E−08 | 4.16E−06 | Proteobacteria; unclassified; unclassified; unclassified |
| OTU3300 | 0.50 | 4.25 | 1.29 | 3.29 | 9.86E−04 | 3.01E−02 | Tenericutes; 94otu23089; 97otu25308; unclassified |
| OTU355 | 12.68 | 3.23 | 0.75 | 4.33 | 1.50E−05 | 1.27E−03 | Bacteroidetes; [Prevotella]; 97otu85617; unclassified |
| OTU405 | 49.59 | −1.77 | 0.53 | −3.32 | 9.14E−04 | 2.99E−02 | Bacteroidetes; Bacteroides; 97otu19740; unclassified |
| OTU408 | 2.93 | 3.59 | 0.85 | 4.21 | 2.54E−05 | 1.76E−03 | Bacteroidetes; Bacteroides; 97otu21727; unclassified |
| OTU420 | 256.66 | 2.57 | 0.63 | 4.09 | 4.37E−05 | 2.78E−03 | Bacteroidetes; Bacteroides; 97otu4177; 24274 |
| OTU447 | 9.34 | −2.58 | 0.73 | −3.51 | 4.45E−04 | 1.79E−02 | Bacteroidetes; Bacteroides; 97otu85586; 58760 |
| OTU460 | 10.75 | 4.75 | 0.71 | 6.70 | 2.11E−11 | 8.06E−09 | Bacteroidetes; Bacteroides; 97otu98467; unclassified |
| OTU664 | 2.20 | 2.46 | 0.70 | 3.53 | 4.20E−04 | 1.78E−02 | Bacteroidetes; 94otu17906; unclassified; unclassified |
| OTU742 | 47.22 | 1.99 | 0.52 | 3.80 | 1.47E−04 | 8.03E−03 | Bacteroidetes; unclassified; unclassified; unclassified |

Abbreviations:
CRC: Colorectal cancer,
SS-UP: Strain Select-UPARSE,
OTU: Operational Taxonomic Unit,
LogFC: Log₂Fold Change,
lfcse; Log₂Fold Change standard error,
stat: Wald test statistic,
pval: p-value associated with Wald test,
padj: FDR adjusted p-value
Base Mean: average of the normalized count values, dividing by size factors
Positive Log₂Fold Change indicates enriched in CRC fecal samples as compared to controls and negative value indicates enriched in control samples as compared to CRC.
“97otu2791” describes a 97% (species-level) OTU cluster for which no standard taxonomic name has been assigned.
Taxonomy follows the phylum; genus; species; strain sequence. For numeric strain annotations please refer to www.secondgenome.com/solutions/resources/data-analysis-tools/strainselect/

TABLE 9
Differential abundance in CRA cases as compared to controls (QIIME-CR)

| | Base | | | | | | |
|---|---|---|---|---|---|---|---|
| OTU | Mean | log2FC | lfcSE | stat | pvalue | padj | Taxonomy |
| OTU1100972 | 69.17 | −1.93 | 0.49 | −3.92 | 8.69E−05 | 6.39E−03 | Firmicutes; Streptococcaceae; Lactococcus; |
| OTU13986 | 1.24 | 2.05 | 0.64 | 3.22 | 1.29E−03 | 3.37E−02 | Firmicutes; Lachnospiraceae; unc; |
| OTU147702 | 101.79 | 1.73 | 0.48 | 3.60 | 3.14E−04 | 1.33E−02 | Firmicutes; Ruminococcaceae; Faecalibacterium; |
| | | | | | | | prausnitzii |
| OTU158310 | 0.69 | 2.79 | 0.67 | 4.17 | 3.07E−05 | 2.93E−03 | Bacteroidetes; Prevotellaceae; Prevotella; |
| OTU1602805 | 9.66 | 1.79 | 0.40 | 4.46 | 8.34E−06 | 1.29E−03 | Firmicutes; Lachnospiraceae; unc; |
| OTU1607319 | 0.86 | 1.55 | 0.51 | 3.02 | 2.52E−03 | 4.91E−02 | Firmicutes; Lachnospiraceae; unc; |
| OTU174571 | 4.75 | 2.16 | 0.48 | 4.47 | 7.91E−06 | 1.29E−03 | Firmicutes;;unc; |
| OTU174654 | 1.74 | −1.76 | 0.47 | −3.72 | 2.01E−04 | 9.60E−03 | Firmicutes; Ruminococcaceae; Ruminococcus; bromii |
| OTU177663 | 240.06 | 1.71 | 0.53 | 3.23 | 1.25E−03 | 3.31E−02 | Firmicutes; Ruminococcaceae; unc; |
| OTU180037 | 52.95 | −1.80 | 0.45 | −3.97 | 7.25E−05 | 5.58E−03 | Firmicutes;;unc; |
| OTU180216 | 34.62 | −2.06 | 0.67 | −3.06 | 2.24E−03 | 4.66E−02 | Firmicutes; Lachnospiraceae; unc; |
| OTU180552 | 7.16 | −1.54 | 0.43 | −3.57 | 3.54E−04 | 1.44E−02 | Firmicutes; Clostridiaceae; unc; |
| OTU180826 | 71.97 | −1.68 | 0.44 | −3.84 | 1.23E−04 | 7.59E−03 | Firmicutes; Ruminococcaceae; Ruminococcus; |
| OTU181871 | 2.01 | 2.35 | 0.51 | 4.59 | 4.33E−06 | 1.18E−03 | Firmicutes; Lachnospiraceae; Dorea; |
| OTU182052 | 13.46 | 2.23 | 0.60 | 3.73 | 1.92E−04 | 9.60E−03 | Bacteroidetes; Bacteroidaceae; Bacteroides; |
| OTU1835779 | 1.72 | 1.91 | 0.40 | 4.83 | 1.39E−06 | 8.87E−04 | Firmicutes; Lachnospiraceae; unc; |
| OTU183579 | 1.30 | 2.04 | 0.57 | 3.56 | 3.74E−04 | 1.49E−02 | Bacteroidetes; Bacteroidaceae; Bacteroides; |
| OTU183686 | 8.25 | 2.13 | 0.57 | 3.77 | 1.60E−04 | 8.87E−03 | Firmicutes; Ruminococcaceae; unc; |
| OTU185864 | 5.22 | 2.31 | 0.50 | 4.62 | 3.78E−06 | 1.18E−03 | Firmicutes; Lachnospiraceae; unc; |
| OTUT86866 | 17.93 | 2.94 | 0.65 | 4.51 | 6.42E−06 | 1.29E−03 | Bacteroidetes; Bacteroidaceae; Bacteroides; |
| OTU1868703 | 3.17 | 2.33 | 0.53 | 4.40 | 1.10E−05 | 1.50E−03 | Firmicutes; Lachnospiraceae; unc; |
| OTU187034 | 1.13 | 1.53 | 0.49 | 3.10 | 1.92E−03 | 4.23E−02 | Firmicutes; Lachnospiraceae; unc; |
| OTU188079 | 25.66 | −1.69 | 0.51 | −3.30 | 9.81E−04 | 2.76E−02 | Firmicutes; Lachnospiraceae; Coprococcus; |
| OTU190058 | 13.75 | −1.65 | 0.43 | −3.81 | 1.40E−04 | 8.11E−03 | Firmicutes; Lachnospiraceae; unc; |
| OTU192963 | 6.27 | −1.56 | 0.47 | −3.33 | 8.58E−04 | 2.61E−02 | Verrucomicrobia; Verrucomicrobiaceae; |
| | | | | | | | Akkermansia; muciniphila |
| OTU193314 | 3.40 | −2.80 | 0.63 | −4.46 | 8.05E−06 | 1.29E−03 | Firmicutes; Ruminococcaceae; Ruminococcus; |
| OTU194151 | 15.82 | −1.53 | 0.44 | −3.44 | 5.85E−04 | 1.96E−02 | Firmicutes;;unc; |
| OTU194758 | 7.13 | −1.66 | 0.44 | −3.72 | 1.99E−04 | 9.60E−03 | Firmicutes; Lachnospiraceae; Coprococcus; |
| OTU194761 | 5.23 | −1.64 | 0.48 | −3.44 | 5.82E−04 | 1.96E−02 | Firmicutes; Lachnospiraceae; unc; |
| OTU1950496 | 5.19 | 2.41 | 0.63 | 3.86 | 1.13E−04 | 7.18E−03 | Bacteroidetes; Bacteroidaceae; Bacteroides; |
| OTU196100 | 12.84 | −1.65 | 0.45 | −3.69 | 2.26E−04 | 1.05E−02 | Firmicutes; Lachnospiraceae; unc; |
| OTU198209 | 4.04 | −1.51 | 0.48 | −3.14 | 1.68E−03 | 3.83E−02 | Firmicutes; Clostridiaceae; SMB53; |
| OTU2046330 | 1.14 | 2.21 | 0.62 | 3.54 | 3.94E−04 | 1.54E−02 | Firmicutes; Lachnospiraceae; unc; |
| OTU2123717 | 5.56 | 1.74 | 0.39 | 4.45 | 8.77E−06 | 1.29E−03 | Firmicutes; Lachnospiraceae; unc; |
| OTU2170530 | 4.22 | 1.58 | 0.48 | 3.30 | 9.67E−04 | 2.76E−02 | Firmicutes; Lachnospiraceae; unc; |
| OTU2250985 | 26.23 | 1.53 | 0.37 | 4.15 | 3.25E−05 | 2.96E−03 | Firmicutes; Lachnospiraceae; Roseburia; |
| OTU230421 | 2.45 | 1.97 | 0.53 | 3.74 | 1.82E−04 | 9.41E−03 | Firmicutes; Ruminococcaceae; unc; |
| OTU2438203 | 2.68 | 1.75 | 0.41 | 4.22 | 2.44E−05 | 2.74E−03 | Firmicutes; Lachnospiraceae; Roseburia; |
| OTU2876801 | 29.62 | −2.89 | 0.69 | −4.17 | 3.01E−05 | 2.93E−03 | Bacteroidetes; Bacteroidaceae; Bacteroides; uniformis |
| OTU290284 | 2.15 | −2.31 | 0.65 | −3.53 | 4.19E−04 | 1.60E−02 | Firmicutes; Ruminococcaceae; unc; |
| OTU3039313 | 21.92 | −4.21 | 0.62 | −6.76 | 1.34E−11 | 2.57E−08 | Firmicutes; Veillonellaceae; Megasphaera; |
| OTU3134492 | 259.86 | 1.71 | 0.37 | 4.61 | 4.01E−06 | 1.18E−03 | Firmicutes; Lachnospiraceae; unc; |
| OTU315223 | 9.03 | 2.16 | 0.66 | 3.29 | 9.91E−04 | 2.76E−02 | Firmicutes; Ruminococcaceae; Anaerotruncus; |
| OTU3186388 | 0.74 | 1.64 | 0.45 | 3.67 | 2.42E−04 | 1.09E−02 | Firmicutes;;unc; |
| OTU3265161 | 14.95 | 1.65 | 0.39 | 4.25 | 2.14E−05 | 2.55E−03 | Firmicutes; Lach nospi raceae; unc; |
| OTU339494 | 37.39 | −2.13 | 0.61 | −3.46 | 5.35E−04 | 1.86E−02 | Firmicutes; Ruminococcaceae; |
| | | | | | | | Faecalibacterium; prausnitzii |
| OTU347639 | 0.48 | −1.52 | 0.50 | −3.04 | 2.34E−03 | 4.67E−02 | Firmicutes; Lachnospiraceae; unc; |
| OTU357930 | 2.32 | 2.16 | 0.68 | 3.19 | 1.40E−03 | 3.44E−02 | Firmicutes; Veillonellaceae; Dialister; |
| OTU359314 | 1.53 | 2.68 | 0.60 | 4.48 | 7.36E−06 | 1.29E−03 | Firmicutes; Ruminococcaceae; Faecalibacterium; prausnitzii |
| OTU3910247 | 0.57 | 2.38 | 0.63 | 3.76 | 1.73E−04 | 9.18E−03 | Bacteroidetes; [Paraprevotellaceae]; [Prevotella]; |
| OTU4094259 | 5.95 | 1.92 | 0.46 | 4.20 | 2.65E−05 | 2.82E−03 | Firmicutes; Ruminococcaceae; unc; |
| OTU4321810 | 38.74 | −2.58 | 0.63 | −4.10 | 4.07E−05 | 3.54E−03 | Firmicutes; Lachnospiraceae; Blautia; |
| OTU4344371 | 2.29 | 1.64 | 0.48 | 3.40 | 6.70E−04 | 2.17E−02 | Proteobacteria; Sphingomonadaceae; Sphingomonas; |
| OTU4355379 | 3.52 | −1.80 | 0.59 | −3.04 | 2.35E−03 | 4.67E−02 | Firmicutes; Lachnospiraceae; [Ruminococcus]; |
| OTU4368484 | 24.06 | −2.48 | 0.64 | −3.88 | 1.05E−04 | 7.15E−03 | Firmicutes; Lachnospiraceae; unc; |
| OTU4372382 | 169.15 | 1.57 | 0.49 | 3.20 | 1.39E−03 | 3.44E−02 | Firmicutes; Lachnospiraceae; unc; |
| OTU4396688 | 349.14 | −1.67 | 0.50 | −3.33 | 8.60E−04 | 2.61E−02 | Firmicutes; Lachnospiraceae; [Ruminococcus]; |
| OTU4401580 | 39.81 | −1.60 | 0.53 | 3.04 | 2.33E−03 | 4.67E−02 | Bacteroidetes; Bacteroidaceae; Bacteroides; |
| OTU4403259 | 0.66 | −1.97 | 0.61 | −3.24 | 1.18E−03 | 3.17E−02 | Actinobacteria; Coriobacteriaceae; unc; |
| OTU4405146 | 8.04 | 2.76 | 0.60 | 4.61 | 3.97E−06 | 1.18E−03 | Firmicutes;;unc; |
| OTU4407515 | 23.48 | 2.04 | 0.67 | 3.04 | 2.33E−03 | 4.67E−02 | Bacteroidetes; Bacteroidaceae; Bacteroides; |
| OTU4415390 | 5.31 | 3.84 | 0.62 | 6.18 | 6.42E−10 | 6.13E−07 | Firmicutes; Lachnospiraceae; unc; |
| OTU4435784 | 3.79 | 2.28 | 0.66 | 3.47 | 5.25E−04 | 1.86E−02 | Bacteroidetes; Bacteroidaceae; Bacteroides; |
| OTU4442899 | 5.35 | 2.45 | 0.63 | 3.90 | 9.62E−05 | 6.81E−03 | Firmicutes;;unc; |
| OTU4447950 | 337.37 | 1.88 | 0.56 | 3.35 | 8.03E−04 | 2.56E−02 | Bacteroidetes; Bacteroidaceae; Bacteroides; |
| OTU4468805 | 1.97 | −2.32 | 0.60 | −3.87 | 1.11E−04 | 7.18E−03 | Firmicutes; Streptococcaceae; Lactococcus; |
| OTU4479443 | 1.65 | 1.66 | 0.45 | 3.66 | 2.55E−04 | 1.11E−02 | Firmicutes; Lachnospiraceae; unc; |
| OTU4483337 | 134.11 | 1.58 | 0.45 | 3.51 | 4.55E−04 | 1.71E−02 | Firmicutes; Lachnospiraceae; unc; |
| OTU518820 | 1.31 | 2.49 | 0.63 | 3.97 | 7.30E−05 | 5.58E−03 | Bacteroidetes; Prevotellaceae; Prevotella; copri |
| OTU54794 | 9.84 | −1.86 | 0.44 | −4.25 | 2.11E−05 | 2.55E−03 | Firmicutes; Streptococcaceae; Streptococcus; |
| OTU798581 | 81.26 | −1.56 | 0.44 | −3.59 | 3.35E−04 | 1.39E−02 | Firmicutes; Ruminococcaceae; Ruminococcus; bromii |
| OTU851733 | 2.94 | 2.11 | 0.66 | 3.20 | 1.38E−03 | 3.44E−02 | Firmicutes; Lactobacillaceae; Lactobacillus; |

Abbreviations:
OTU: Operational Taxonomic Unit,
LogFC: Log₂Fold Change,
lfcse: Log₂Fold Change standard error,
stat: Wald test statistic,
pval: p-value associated with Wald test,
padj: FDR adjusted p-value, unclassified
Base Mean: average of the normalized count values, dividing by size factors
Positive Log₂Fold Change indicates enriched in CRA fecal samples as compared to controls and negative value indicates enriched in control samples as compared to CRA.

OTUs within the genera Ruminococcus and Lactobacillus, and the family Enterobacteriaceae were consistently enriched in both CRC and CRA cases relative to controls. In particular, Fusobacterium sp. was enriched in CRC cases but not among CRA cases.

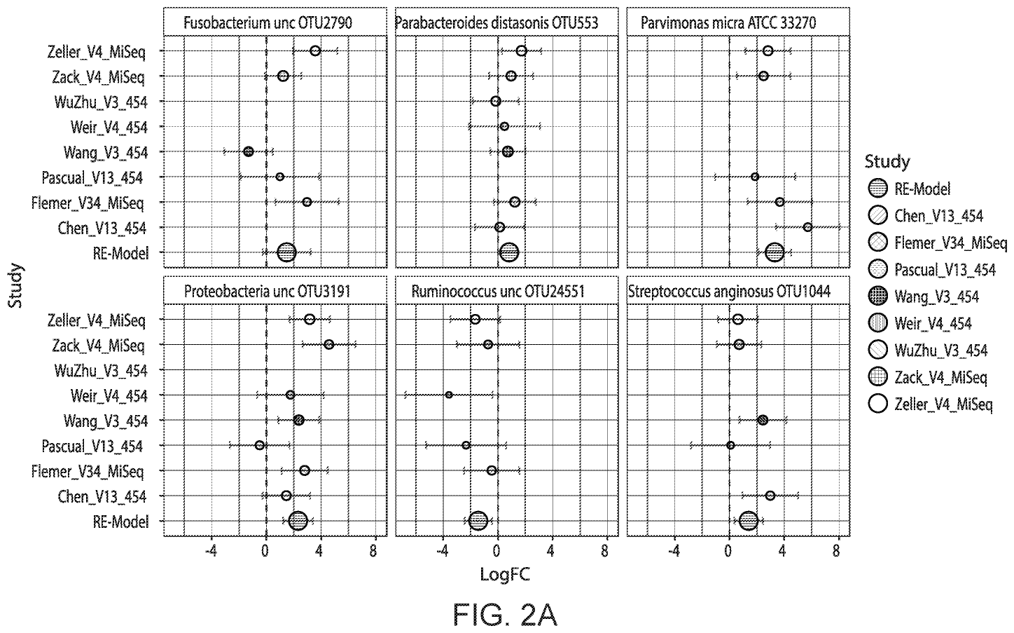

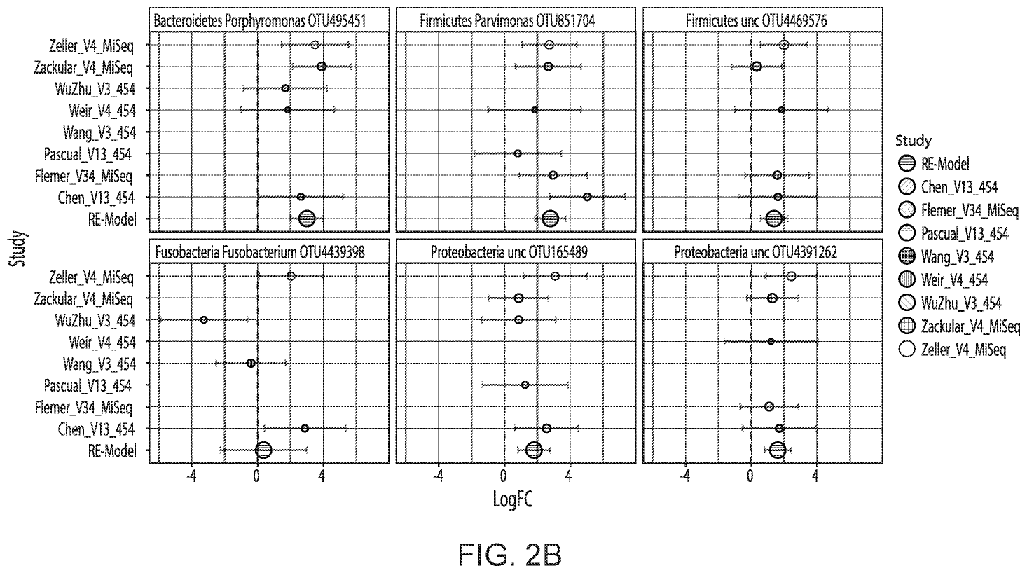

We built an REM to evaluate the degree to which microbial markers of disease were consistent across studies. A total of 142 OTUs from the SS-UP pipeline and 388 OTUs by the QIIMF-CR pipeline occurred in five or more studies. The strain Parvimonas micra ATCC 33270 was significantly elevated in CRC cases, relative to controls, in five out of the eight studies by SS-UP (adjusted REM log₂fold estimate: 3.3 95% CI:2.2-4.5, REM p<0.001, FDR adjusted p-value <0.001). Other examples from the SS-UP pipeline include OTUs within Proteobacteria (adjusted REM log₂fold estimate across 8 studies: 1.96, 95% CI: 0.8, 3.1, REM p 0.001. FDR p=0.07) and Streptococcus anginosus (adjusted REM log₂fold estimate across 5 studies: 1.4, 95% CI: 0.4, 2.4, REM p-value: 0.008, FDR p: 0.19). Despite the biological and technical heterogeneity associated with these studies, the above markers emerged as significant signals for CRC (FIG. 2A; Table 10)

TABLE 10
Differentially abundant OTUs in CRC cases as compared to controls identified by the
Random Effects Model (REM) for the SS-UP. Taxonomy follows the convention of phylum,
genus, species, strain sequence. For strain numeric annotations, please refer to
www.secondgenome.com/solutions/resources/data-amlysis-tools/strainselect/

| | | | | | | | | | | |
|---|---|---|---|---|---|---|---|---|---|---|
| Study | LogFC | 95% CI | p | τ² | SE τ² | QE | QEp | I² | H² | FDR |

Firmicutes; Parvimonas; 97otu12932; 72331

| | | | | | | | | | | |
|---|---|---|---|---|---|---|---|---|---|---|
| RE-Model | 3.31 | 2.12; 4.50 | 0.00 | 0.66 | 1.30 | 6.45 | 0.17 | 36.10 | 1.56 | 7.28E−06 |
| Zack_V4_MiSeq | 2.49 | 0.53; 4.45 | | | | | | | | |
| Chen_V13_454 | 5.73 | 3.40; 8.05 | | | | | | | | |
| Zeller_V4_MiSeq | 2.82 | 1.17; 4.47 | | | | | | | | |
| Flemer_V34_MiSeq | 3.68 | 1.33; 6.03 | | | | | | | | |
| Pascual_V13_454 | 1.87 | −1.06; 4.80 | | | | | | | | |

Proteobacteria; unclassified; unclassified; unclassified

| | | | | | | | | | | |
|---|---|---|---|---|---|---|---|---|---|---|
| RE-Model | 1.96 | 0.79; 3.13 | 0.00 | 2.00 | 1.52 | 22.92 | 0.00 | 71.35 | 3.49 | 7.34E−02 |
| Zack_V4_MiSeq | 4.58 | 2.65; 6.51 | | | | | | | | |
| WuZhu_V3_454 | −0.43 | −2.33; 1.47 | | | | | | | | |
| Wang_V3_454 | 2.37 | 0.87; 3.87 | | | | | | | | |
| Chen_V13_454 | 1.46 | −0.28; 3.19 | | | | | | | | |
| Zeller_V4_MiSeq | 3.17 | 1.70; 4.64 | | | | | | | | |
| Weir_V4_454 | 1.76 | −0.67; 4.19 | | | | | | | | |
| Flemer_V34_MiSeq | 2.80 | 1.12; 4.48 | | | | | | | | |
| Pascual_V13_454 | −0.49 | −2.67; 1.68 | | | | | | | | |

Firmicutes; Streptococcus; anginosus; unclassified

| | | | | | | | | | | |
|---|---|---|---|---|---|---|---|---|---|---|
| RE-Model | 1.40 | 0.37; 2.44 | 0.01 | 0.54 | 0.97 | 6.60 | 0.16 | 39.02 | 1.64 | 1.86E−01 |
| Zack_V4_MiSeq | 0.71 | −0.91; 2.33 | | | | | | | | |
| Wang_V3_454 | 2.44 | 0.73; 4.16 | | | | | | | | |
| Chen_V13_454 | 2.98 | 0.94; 5.02 | | | | | | | | |
| Zeller_V4_MiSeq | 0.62 | −0.83; 2.07 | | | | | | | | |
| Pascual_V13_454 | 0.08 | −2.80; 2.97 | | | | | | | | |

Firmicutes; 94otu3610; 97otu8133; unclassified

| | | | | | | | | | | |
|---|---|---|---|---|---|---|---|---|---|---|
| RE-Model | −1.21 | −2.13; −0.30 | 0.01 | 0.33 | 0.82 | 7.26 | 0.20 | 25.28 | 1.34 | 1.86E−01 |
| Zack_V4_MiSeq | −2.83 | −4.75; −0.91 | | | | | | | | |
| WuZhu_V3_454 | −0.40 | −2.78; 1.98 | | | | | | | | |
| Wang_V3_454 | −1.57 | −3.56; 0.43 | | | | | | | | |
| Zeller_V4_MiSeq | −1.33 | −2.87; 0.21 | | | | | | | | |
| Flemer_V34_MiSeq | −1.37 | −3.28; 0.55 | | | | | | | | |
| Pascual_V13_454 | 1.06 | −1.28; 3.40 | | | | | | | | |

Firmicutes; Ruminococcus; 97otu15279; unclassified

| | | | | | | | | | | |
|---|---|---|---|---|---|---|---|---|---|---|
| RE-Model | −1.44 | −2.44; −0.44 | 0.00 | 0.00 | 0.90 | 3.60 | 0.46 | 0.00 | 1.00 | 1.86E−01 |
| Zack_V4_MiSeq | −0.72 | −3.00; 1.56 | | | | | | | | |
| Zeller_V4_MiSeq | −1.66 | −3.47; 0.15 | | | | | | | | |
| Weir_V4_454 | −3.58 | −6.76; −0.40 | | | | | | | | |
| Flemer_V34_MiSeq | −0.46 | −2.47; 1.56 | | | | | | | | |
| Pascual_V13_454 | −2.33 | −5.25; 0.59 | | | | | | | | |

Firmicutes; [Eubacterium]; dolichum; unclassified

| | | | | | | | | | | |
|---|---|---|---|---|---|---|---|---|---|---|
| RE-Model | 1.00 | 0.28; 1.72 | 0.01 | 0.00 | 0.52 | 4.52 | 0.61 | 0.00 | 1.00 | 1.86E−01 |
| Zack_V4_MiSeq | 0.17 | −1.48; 1.82 | | | | | | | | |
| Wang_V3_454 | 1.94 | 0.28; 3.60 | | | | | | | | |
| Chen_V13_454 | 0.15 | −2.10; 2.40 | | | | | | | | |
| Zeller_V4_MiSeq | 1.79 | 0.23; 3.35 | | | | | | | | |
| Flemer_V34_MiSeq | 0.25 | −1.67; 2.16 | | | | | | | | |
| Pascual_V13_454 | 0.97 | −1.97; 3.91 | | | | | | | | |

Bacteroidetes; Parabacteroides; distasonis; unclassified

| | | | | | | | | | | |
|---|---|---|---|---|---|---|---|---|---|---|
| RE-Model | 0.82 | 0.23; 1.42 | 0.01 | 0.00 | 0.36 | 3.96 | 0.68 | 0.00 | 1.00 | 1.86E−01 |
| Zack_V4_MiSeq | 0.96 | −0.65; 2.57 | | | | | | | | |
| WuZhu_V3_454 | −0.16 | −1.83; 1.52 | | | | | | | | |
| Wang_V3_454 | 0.72 | −0.56; 2.00 | | | | | | | | |
| Chen_V13_454 | 0.13 | −1.67; 1.94 | | | | | | | | |
| Zeller_V4_MiSeq | 1.73 | 0.29; 3.16 | | | | | | | | |
| Weir_V4_454 | 0.48 | −2.12; 3.08 | | | | | | | | |
| Flemer_V34_MiSeq | 1.23 | −0.30; 2.76 | | | | | | | | |

Bacteroidetes; Prevotella; copri; unclassified

| | | | | | | | | | | |
|---|---|---|---|---|---|---|---|---|---|---|
| RE-Model | −1.52 | −2.76; −0.28 | 0.02 | 1.68 | 1.61 | 15.18 | 0.02 | 61.95 | 2.63 | 2.88E−01 |
| Zack_V4_MiSeq | −1.28 | −2.96; 0.39 | | | | | | | | |
| WuZhu_V3_454 | −1.83 | −4.41; 0.76 | | | | | | | | |
| Wang_V3_454 | −1.11 | −3.07; −0.85 | | | | | | | | |
| Zeller_V4_MiSeq | 0.34 | −1.20; 1.88 | | | | | | | | |
| Weir_V4_454 | −5.65 | −8.36; −2.94 | | | | | | | | |
| Flemer_V34_MiSeq | −0.93 | −2.86; 1.00 | | | | | | | | |
| Pascual_V13_454 | −1.63 | −4.21; 0.95 | | | | | | | | |

Firmicutes; Coprococcus; eutactus; 38993

| | | | | | | | | | | |
|---|---|---|---|---|---|---|---|---|---|---|
| RE-Model | −1.02 | −1.92; −0.13 | 0.03 | 0.00 | 0.74 | 3.17 | 0.53 | 0.00 | 1.00 | 2.92E−01 |
| Zack_V4_MiSeq | −0.60 | −2.94; 1.73 | | | | | | | | |
| Chen_V13_454 | −2.67 | −5.10; −0.25 | | | | | | | | |
| Zeller_V4_MiSeq | −1.24 | −2.84; 0.36 | | | | | | | | |
| Flemer_V34_MiSeq | −0.71 | −2.57; 1.15 | | | | | | | | |
| Pascual_V13_454 | 0.24 | −2.29; 2.77 | | | | | | | | |

Proteobacteria; Sutterella; 97otu21533; unclassified

| | | | | | | | | | | |
|---|---|---|---|---|---|---|---|---|---|---|
| RE-Model | 1.59 | 0.22; 2.95 | 0.02 | 1.04 | 1.71 | 7.08 | 0.13 | 43.20 | 1.76 | 2.92E−01 |
| Zack_V4_MiSeq | 4.45 | 1.83; 7.07 | | | | | | | | |
| WuZhu_V3_454 | 0.90 | −1.73; 3.54 | | | | | | | | |
| Zeller_V4_MiSeq | 0.32 | −1.56; 2.19 | | | | | | | | |
| Flemer_V34_MiSeq | 1.75 | −0.23; 3.73 | | | | | | | | |
| Pascual_V13_454 | 0.93 | −1.99; 3.84 | | | | | | | | |

Verrucomicrobia; Akkermansia; muciniphila; unclassified

| | | | | | | | | | | |
|---|---|---|---|---|---|---|---|---|---|---|
| RE-Model | 1.16 | 0.14; 2.18 | 0.03 | 0.65 | 1.02 | 8.26 | 0.14 | 40.43 | 1.68 | 2.92E−01 |
| Zack_V4_MiSeq | 0.84 | −0.91; 2.59 | | | | | | | | |
| Wang_V3_454 | 1.79 | −0.59; 4.17 | | | | | | | | |
| Chen_V13_454 | −0.29 | −2.97; 2.39 | | | | | | | | |
| Zeller_V4_MiSeq | 2.35 | 0.90; 3.80 | | | | | | | | |
| Weir_V4_454 | 2.36 | −0.04; 4.76 | | | | | | | | |
| Flemer_V34_MiSeq | −0.31 | −2.01; 1.39 | | | | | | | | |

Bacteroidetes; Bacteroides; 97otu85586; 58760

| | | | | | | | | | | |
|---|---|---|---|---|---|---|---|---|---|---|
| RE-Model | −1.81 | −3.41; −0.21 | 0.03 | 1.68 | 2.34 | 8.05 | 0.09 | 51.62 | 2.07 | 2.92E−01 |
| Zack_V4_MiSeq | −5.05 | −7.78; −2.33 | | | | | | | | |
| Zeller_V4_MiSeq | −1.75 | −3.52; 0.02 | | | | | | | | |
| Weir_V4_454 | −1.43 | −4.67; 1.82 | | | | | | | | |
| Flemer_V34_MiSeq | −0.84 | −3.25; 1.58 | | | | | | | | |
| Pascual_V13_454 | 0.07 | −2.83; 2.96 | | | | | | | | |

Bacteroidetes; Prevotella; unclassified; unclassified

| | | | | | | | | | | |
|---|---|---|---|---|---|---|---|---|---|---|
| RE-Model | −0.93 | −1.75; −0.12 | 0.02 | 0.10 | 0.67 | 8.50 | 0.20 | 8.01 | 1.09 | 2.92E−01 |
| Zack_V4_MiSeq | 0.03 | −1.87; 1.93 | | | | | | | | |
| WuZhu_V3_454 | −2.18 | −4.45; 0.09 | | | | | | | | |
| Wang_V3_454 | −1.31 | −3.07; 0.45 | | | | | | | | |
| Zeller_V4_MiSeq | −1.20 | −2.71; 0.31 | | | | | | | | |
| Weir_V4_454 | −3.37 | −6.37; −0.37 | | | | | | | | |
| Flemer_V34_MiSeq | 0.92 | −1.66; 3.50 | | | | | | | | |
| Pascual_V13_454 | 0.67 | | | | | | | | | |

Firmicutes; 94otu20757; 97otu25367; unclassified

| | | | | | | | | | | |
|---|---|---|---|---|---|---|---|---|---|---|
| RE-Model | 0.83 | 0.06; 1.61 | 0.04 | 0.00 | 0.58 | 4.03 | 0.55 | 0.00 | 1.00 | 3.62E−01 |
| Zack_V4_MiSeq | 0.22 | −1.75; 2.18 | | | | | | | | |
| WuZhu_V3_454 | −0.82 | −2.97; 1.34 | | | | | | | | |
| Chen_V13_454 | 0.67 | −1.73; 3.07 | | | | | | | | |
| Zeller_V4_MiSeq | 1.48 | −0.07; 3.03 | | | | | | | | |
| Flemer_V34_MiSeq | 1.26 | −0.45; 2.98 | | | | | | | | |
| Pascual_V13_454 | 1.48 | 0.81; 3.76 | | | | | | | | |

Bacteroidetes; Porphyromonas; 97otu52506; unclassified

| | | | | | | | | | | |
|---|---|---|---|---|---|---|---|---|---|---|
| RE-Model | 2.56 | 0.12; 5.00 | 0.04 | 6.40 | 5.48 | 20.28 | 0.00 | 83.09 | 5.91 | 3.79E−01 |
| Zack_V4_MiSeq | 4.57 | 2.55; 6.58 | | | | | | | | |
| WuZhu_V3_454 | −2.34 | −5.09; 0.41 | | | | | | | | |
| Chen_V13_454 | 4.84 | 2.33; 7.35 | | | | | | | | |
| Zeller_V4_MiSeq | 2.16 | 0.22; 4.10 | | | | | | | | |
| Flemer_V34_MiSeq | 2.24 | 0.85; 5.62 | | | | | | | | |

Fusobacteria; Fusobacterium; unclassified; unclassified

| | | | | | | | | | | |
|---|---|---|---|---|---|---|---|---|---|---|
| RE-Model | 1.61 | 0.04; 3.17 | 0.04 | 3.24 | 2.56 | 26.98 | 0.00 | 74.84 | 3.97 | 3.93E−01 |
| Zack_V4_MiSeq | 3.83 | 2.15; 5.50 | | | | | | | | |
| WuZhu_V3_454 | 0.56 | −1.98; 3.09 | | | | | | | | |
| Wang_V3_454 | −1.31 | −3.08; 0.46 | | | | | | | | |
| Chen_V13_454 | 0.04 | −2.65; 2.72 | | | | | | | | |
| Zeller_V4_MiSeq | 3.57 | 1.95; 5.19 | | | | | | | | |
| Flemer_V34_MiSeq | 2.97 | 0.65; 5.29 | | | | | | | | |
| Pascual_V13_454 | 0.09 | 1.99; 2.87 | | | | | | | | |

Bacteroidetes; Bacteroides; plebeius; 4836

| | | | | | | | | | | |
|---|---|---|---|---|---|---|---|---|---|---|
| RE-Model | −1.40 | −2.79; −0.02 | 0.05 | 2.23 | 2.01 | 19.47 | 0.00 | 65.46 | 2.89 | 3.96E−01 |
| Zack_V4_MiSeq | −4.84 | −6.65; −3.03 | | | | | | | | |
| WuZhu_V3_454 | −1.55 | −4.13; 1.03 | | | | | | | | |
| Wang_V3_454 | −0.53 | −2.46; 1.40 | | | | | | | | |
| Chen_V13_454 | −0.45 | −3.22; 2.32 | | | | | | | | |
| Zeller_V4_MiSeq | 0.17 | −1.50; 1.84 | | | | | | | | |
| Flemer_V34_MiSeq | −0.80 | −3.23; 1.63 | | | | | | | | |
| Pascual_V13_454 | 1.55 | 4.20; 1.10 | | | | | | | | |

Abbreviations:
LogFC: Log₂Fold Change,
τ2: The (total) amount of heterogeneity among the true effects,
SE: Standard error,
QE: Test statistic for the test of (residual) heterogeneity from the full model,
QEp: p-value associated with QE,
I2: For a random-effects model, I2 estimates (in percent) how much of the total variability in the effect size estimates (which is composed of heterogeneity plus sampling variability) can be attributed to heterogeneity among the true effects,
H2: estimates the ratio of the total amount of variability in the effect size estimates to the amount of sampling variability,
FDR: False Discovery Rate,
RE: Random Effects

Fusobacterium sp. was detected in seven of the eight CRC-microbiome association studies, but it did not differ consistently between cases and controls. In some studies, little difference was observed, and in others inverse relationships were detected (i.e., abundant in controls relative to cases). The enrichment of Fusobacterium sp in cases relative to controls was observed particularly in the MiSeq studies, leading to an adjusted REM estimate of 1.6 (95% CI: 0.04, 3.2, p: 0.04, FDR p: 0.4) (Table 10).

Taxa determined significant by the REM were concordant with box-plots of the relative abundance distribution of these taxa across studies however sparsely distributed in the comparison groups. The QIIME-CR pipeline also identified multiple OTUs that were consistently enriched or depleted in cases relative to controls, but only a few had high-confidence species-level taxonomic assignments. One such example was an OTU within the genus Porphyrmonas (adjusted REM log 2fold estimate across 5 studies: 2.9, 95% CI: 2.0, 3.9, REM p-value: 2.2*10-9. FDR p: 5.8*10-7) (FIG. 2B; Table 11).

TABLE 11
Differentially abundant OTUs in CRC cases as compared to controls identified by the
REM (QIIME-CR). Taxonomy follows the convention of: phylum, genus, species. Blanks are
given in cases of uncertain classification at a given taxonomic rank.

| | | | | | | | | | | |
|---|---|---|---|---|---|---|---|---|---|---|
| | | | | | τ² | | | | | |
| Study | LogFC | 95% CI | p | τ² | SE | QE | QEp | I² | H² | FDR |

Bacteroidetes; Porphyromonas;

| | | | | | | | | | | |
|---|---|---|---|---|---|---|---|---|---|---|
| RE-Model | 3.00 | 2.02; 3.98 | 2.28E−09 | 0.0 | 0.88 | 3.12 | 0.54 | 0.0 | 1.00 | 5.81E−07 |
| Zack_V4_MiSeq | 3.90 | 2.17; 5.64 | | | | | | | | |
| WuZhu_V3_454 | 1.68 | −0.80; 4.17 | | | | | | | | |
| Chen_V13_454 | 2.63 | 0.10; 5.15 | | | | | | | | |
| Zeller_V4_MiSeq | 3.49 | 1.50; 5.48 | | | | | | | | |
| Weir_V4_454 | 1.83 | −0.93; 4.59 | | | | | | | | |

Firmicutes; Parvimonas;

| | | | | | | | | | | |
|---|---|---|---|---|---|---|---|---|---|---|
| RE-Model | 2.79 | 1.87; 3.71 | 3.00E−09 | 0.15 | 0.82 | 6.64 | 0.25 | 11.65 | 1.13 | 5.81E−07 |
| Zack_V4_MiSeq | 2.66 | 0.71; 4.60 | | | | | | | | |
| Chen_V13_454 | 5.04 | 2.80; 7.27 | | | | | | | | |
| Zeller_V4_MiSeq | 2.72 | 1.09; 4.36 | | | | | | | | |
| Weir_V4_454 | 1.83 | −0.93; 4.59 | | | | | | | | |
| Flemer_V34_MiSeq | 2.95 | 0.90; 5.00 | | | | | | | | |
| Pascual_V13_454 | 0.81 | −1.78; 3.40 | | | | | | | | |

Proteobacteria;

| | | | | | | | | | | |
|---|---|---|---|---|---|---|---|---|---|---|
| RE-Model | 1.61 | 0.80; 2.41 | 8.74E−05 | 0.00 | 0.57 | 1.74 | 0.78 | 0.00 | 1.00 | 1.13E−02 |
| Zack_V4_MiSeq | 1.28 | −0.23; 2.78 | | | | | | | | |
| Chen_V13_454 | 1.70 | −0.49; 3.88 | | | | | | | | |
| Zeller_V4_MiSeq | 2.43 | 0.91; 3.95 | | | | | | | | |
| Weir_V4_454 | 1.20 | −1.57; 3.97 | | | | | | | | |
| Flemer_V34_MiSeq | 1.09 | −0.62; 2.80 | | | | | | | | |

Proteobacteria;

| | | | | | | | | | | |
|---|---|---|---|---|---|---|---|---|---|---|
| RE-Model | 1.79 | 0.82; 2.77 | 3.13E−04 | 0.19 | 0.87 | 4.36 | 0.36 | 15.05 | 1.18 | 3.04E−02 |
| Zack_V4_MiSeq | 0.87 | −0.90; 2.63 | | | | | | | | |
| WuZhu_V3_454 | 0.86 | −1.34; 3.07 | | | | | | | | |
| Chen_V13_454 | 2.56 | 0.69; 4.43 | | | | | | | | |
| Zeller_V4_MiSeq | 3.08 | 1.20; 4.97 | | | | | | | | |
| Pascual_V13_454 | 1.26 | −1.28; 3.80 | | | | | | | | |

OTU4469576, Firmicutes;

| | | | | | | | | | | |
|---|---|---|---|---|---|---|---|---|---|---|
| RE-Model | 1.38 | 0.56; 2.20 | 9.48E−04 | 0.02 | 0.60 | 2.77 | 0.59 | 2.79 | 1.03 | 7.36E−02 |
| Zack_V4_MiSeq | 0.33 | −1.17; 1.83 | | | | | | | | |
| Chen_V13_454 | 1.61 | −0.73; 3.96 | | | | | | | | |
| Zeller_V4_MiSeq | 1.98 | 0.59; 3.38 | | | | | | | | |
| Weir_V4_454 | 1.83 | −0.93; 4.59 | | | | | | | | |
| Flemer_V34_MiSeq | 1.57 | −0.33; 3.47 | | | | | | | | |

Firmicutes; Blautia;

| | | | | | | | | | | |
|---|---|---|---|---|---|---|---|---|---|---|
| RE-Model | −1.26 | −2.14; −0.38 | 4.89E−03 | 0.00 | 0.69 | 1.12 | 0.89 | 0.00 | 1.00 | 2.74E−01 |
| Zack_V4_MiSeq | −1.24 | −3.22; 0.74 | | | | | | | | |
| WuZhu_V3_454 | −0.28 | −2.86; 2.30 | | | | | | | | |
| Wang_V3_454 | −1.03 | −2.89; 0.84 | | | | | | | | |
| Zeller_V4_MiSeq | −1.78 | −3.23; −0.32 | | | | | | | | |
| Weir_V4_454 | −1.05 | −3.82; 1.72 | | | | | | | | |

Proteobacteria; Sutterella;

| | | | | | | | | | | |
|---|---|---|---|---|---|---|---|---|---|---|
| RE-Model | −1.33 | −2.32; −0.34 | 8.25E−03 | 0.00 | 0.89 | 1.81 | 0.77 | 0.00 | 1.00 | 2.91E−01 |
| Zack_V4_MiSeq | −1.19 | −3.76; 1.39 | | | | | | | | |
| WuZhu_V3_454 | −2.91 | −5.51; −0.31 | | | | | | | | |
| Wang_V3_454 | −1.03 | −2.97; 0.91 | | | | | | | | |
| Zeller_V4_MiSeq | −1.37 | −3.60; 0.87 | | | | | | | | |
| Flemer_V34_MiSeq | −0.80 | −2.78; 1.18 | | | | | | | | |

Bacteroidetes; Bacteroides;

| | | | | | | | | | | |
|---|---|---|---|---|---|---|---|---|---|---|
| RE-Model | −1.31 | −2.54; −0.08 | 3.71E−02 | 1.01 | 1.49 | 8.87 | 0.11 | 43.3 | 1.76 | 4.26E−01 |
| Zack_V4_MiSeq | −4.63 | −7.11; −2.14 | | | | | | | | |
| WuZhu_V3_454 | −0.05 | −2.34; 2.24 | | | | | | | | |
| Zeller_V4_MiSeq | −0.55 | −2.29; 1.18 | | | | | | | | |
| Weir_V4_454 | −1.05 | −3.82; 1.72 | | | | | | | | |
| Flemer_V34_MiSeq | −0.97 | −3.04; 1.09 | | | | | | | | |
| Pascual_V13_454 | −1.11 | −3.64; 1.42 | | | | | | | | |

Bacteroidetes; Paraprevotella;

| | | | | | | | | | | |
|---|---|---|---|---|---|---|---|---|---|---|
| RE-Model | −1.03 | −1.90; −0.16 | 2.00E−02 | 0.00 | 0.73 | 4.79 | 0.44 | 0.0 | 1.0 | 4.26E−01 |
| WuZhu_V3_454 | 0.38 | −2.12; 2.88 | | | | | | | | |
| Wang_V3_454 | −2.49 | −4.36; −0.61 | | | | | | | | |
| Chen_V13_454 | −0.17 | −2.69; 2.35 | | | | | | | | |
| Zeller_V4_MiSeq | −0.76 | −2.58; 1.07 | | | | | | | | |
| Flemer_V34_MiSeq | −0.61 | −2.50; 1.28 | | | | | | | | |
| Pascual_V13_454 | −1.99 | −4.57; 0.60 | | | | | | | | |

Firmicutes; Coprococcus;

| | | | | | | | | | | |
|---|---|---|---|---|---|---|---|---|---|---|
| RE-Model | −0.87 | −1.60; −0.13 | 2.05E−02 | 0.00 | 0.47 | 1.53 | 0.82 | 0.0 | 1.0 | 4.26E−01 |
| Zack_V4_MiSeq | −0.09 | −1.65; 1.47 | | | | | | | | |
| Zeller_V4_MiSeq | −1.37 | −2.71; −0.03 | | | | | | | | |
| Weir_V4_454 | −1.05 | −3.82; 1.72 | | | | | | | | |
| Flemer_V34_MiSeq | −0.92 | −2.21; 0.36 | | | | | | | | |
| Pascual_V13_454 | −0.75 | −3.34; 1.83 | | | | | | | | |

Firmicutes; Ruminococcus;

| | | | | | | | | | | |
|---|---|---|---|---|---|---|---|---|---|---|
| RE-Model | −1.11 | −2.12; −0.09 | 3.23E−02 | 0.00 | 0.94 | 2.86 | 0.58 | 0.0 | 1.0 | 4.26E−01 |
| WuZhu_V3_454 | −0.03 | −2.41; 2.34 | | | | | | | | |
| Wang_V3_454 | −0.65 | −2.95; 1.64 | | | | | | | | |
| Chen_V13_454 | −1.85 | −4.38; 0.68 | | | | | | | | |
| Zeller_V4_MiSeq | −2.33 | −4.42; −0.25 | | | | | | | | |
| Flemer_V34_MiSeq | −0.55 | −2.70; 1.60 | | | | | | | | |

Bacteroidetes; Bacteroides;

| | | | | | | | | | | |
|---|---|---|---|---|---|---|---|---|---|---|
| RE-Model | 1.70 | 0.07; 3.33 | 4.12E−02 | 2.89 | 2.62 | 15.19 | 0.01 | 70.74 | 3.42 | 4.26E−01 |
| Zack_V4_MiSeq | 2.99 | 1.08; 4.90 | | | | | | | | |
| WuZhu_V3_454 | −1.28 | −3.86; 1.29 | | | | | | | | |
| Chen_V13_454 | 0.54 | −1.94; 3.02 | | | | | | | | |
| Zeller_V4_MiSeq | 1.19 | −0.52; 2.91 | | | | | | | | |
| Weir_V4_454 | 5.31 | 2.65; 7.98 | | | | | | | | |
| Flemer_V34_MiSeq | 1.49 | −0.45; 3.43 | | | | | | | | |

Firmicutes; Blautia;

| | | | | | | | | | | |
|---|---|---|---|---|---|---|---|---|---|---|
| RE-Model | 1.22 | 0.13; 2.30 | 2.76E−02 | 0.26 | 1.08 | 4.52 | 0.34 | 17.12 | 1.21 | 4.26E−01 |
| Zack_V4_MiSeq | 2.79 | 0.71; 4.88 | | | | | | | | |
| aChen_V13_454 | 0.25 | −2.01; 2.52 | | | | | | | | |
| Zeller_V4_MiSeq | 1.71 | −0.28; 3.70 | | | | | | | | |
| Weir_V4_454 | 1.20 | −1.57; 3.97 | | | | | | | | |
| Flemer_V34_MiSeq | −0.05 | −2.16; 2.05 | | | | | | | | |

Bacteroidetes; Bacteroides; uniformis

| | | | | | | | | | | |
|---|---|---|---|---|---|---|---|---|---|---|
| RE-Model | −0.84 | −1.54; −0.15 | 1.75E−02 | 0.00 | 0.47 | 2.96 | 0.70 | 0.00 | 1.00 | 4.26E−01 |
| Zack_V4_MiSeq | −0.23 | −1.82; 1.37 | | | | | | | | |
| WuZhu_V3_454 | −1.09 | −2.74; 0.56 | | | | | | | | |
| Wang_V3_454 | −1.23 | −2.88; 0.43 | | | | | | | | |
| Chen_V13_454 | −1.33 | −3.31; 0.66 | | | | | | | | |
| Flemer_V34_MiSeq | −1.25 | −2.71; 0.21 | | | | | | | | |
| Pascual_V13_454 | 0.59 | −1.60; 2061 | | | | | | | | |

LogFC: Log₂Fold Ch
Abbreviations:
LogFC: Log₂Fold Change,
τ2: The (total) amount of heterogeneity among the true effects,
SE: Standard error,
QE: Test statistic for the test of (residual) heterogeneity from the full model,
QEp: p-value associated with QE,
I²: For a random-effects model,
I² estimates (in percent) how much of the total variability in the effect size estimates (which is composed of heterogeneity plus sampling variability) can be attributed to heterogeneity among the true effects,
H²: estimates the ratio of the total amount of variability in the effect size estimates to the amount of sampling variability,
FDR: False Discovery Rate,
RE: Random Effects

A similar REM was built for the four studies that had CRA and controls. The SS-UP pipeline identified 192 OTUs that were detected in either 3 or all 4 of the CRA-containing studies. OTUs within the family Lachnospiraceae (OTU1642 adjusted REM estimate: −1.96, 95% CI: −2.97, —0.94, p: 1.5*10-4, FDR: 0.03), and species Bacteroides plebius (adjusted REM estimate: 1.86, 95% CI: 0.5-3.2, p: 0.005, FDR: 0.48) were detected in three of the four CRA studies and had a high adjusted REM log 2fold change but were not statistically significant after FDR correction. Likewise, the QIIME-CR pipeline produced OTUs within the genera Bacteroides (adjusted REM estimate: −2.9, 95% CI: −4.1, −1.7, p: 2.9*10-6, FDR: 0.001) and Ruminococcus (adjusted REM estimate 1.8, 95% CI: 0.6, 2.9, p: 0.0.003, FDR: 0.5) (Tables 12 and 13).

TABLE 12
Differentially abundant OTUs in CRA cases as compared to controls identified by the
Random Effects model (SS-UP). Taxonomy follows the convention of phylum, genus, species,
strain sequence. For strain numeric annotations, please refer to
www.secondgenome.com/solutions/resources/data-analysis-tools/strainselect/

| | | Log | CILB; | | | SE_T | | | | |
|---|---|---|---|---|---|---|---|---|---|---|
| OTUID | Study | FC | CIUB | p | tau | au 2 | I² | H2 | FDR | Taxonomy |
| OTU1642 | RE-Model | −1.96 | −2.97; | 1.51E−04 | 0 | 0.81 | 0 | 1 | 0.027 | Firmicutes; 94otu12657; 97otu23541; |
| | | | −0.94 | | | | | | | unclassified |
| OTU1642 | Zackular_V4_MiSeq | −2.70 | −4.33; | 1.51E−04 | | | | | 0.027 | Firmicutes; 94otu12657; 97otu23541; |
| | | | −1.07 | | | | | | | unclassified |
| OTU1642 | Pascual_V13_454 | −1.58 | −4.61; | 1.51E−04 | | | | | 0.027 | Firmicutes; 94otu12657; 97otu23541; |
| | | | 1.44 | | | | | | | unclassified |
| OTU1642 | Zeller_V4_MiSeq | −1.43 | −2.92; | 1.51E−04 | | | | | 0.027 | Firmicutes; 94otu12657; 97otu23541; |
| | | | 0.06 | | | | | | | unclassified |
| OTU1375 | RE-Model | 1.95 | 0.73; | 1.66E−03 | 0 | 1.17 | 0 | 1 | 0.150 | Firmicutes; 94otu15016; 97otu26208; |
| | | | 3.16 | | | | | | | unclassified |
| OTU1375 | Zackular_V4_MiSeq | 2.43 | 0.31; | 1.66E−03 | | | | | 0.150 | Firmicutes; 94otu15016; 97otu26208; |
| | | | 4.55 | | | | | | | unclassified |
| OTU1375 | Pascual_V13_454 | 2.00 | −0.82; | 1.66E−03 | | | | | 0.150 | Firmicutes; 94otu15016; 97otu26208; |
| | | | 4.81 | | | | | | | unclassified |
| OTU1375 | Zeller_V4_MiSeq | 1.57 | −0.25; | 1.66E−03 | | | | | 0.150 | Firmicutes; 94otu15016; 97otu26208; |
| | | | 3.40 | | | | | | | unclassified |
| OTU3191 | RE-Model | 1.51 | 0.48; | 4.18E−03 | 7.58E−06 | 0.87 | <0.001 | 1 | 0.252 | Proteobacteria; unclassified; unclassified; |
| | | | 2.54 | | | | | | | unclassified |
| OTU3191 | Zackular_V4_MiSeq | 1.76 | −0.13; | 4.18E−03 | | | | | 0.252 | Proteobacteria; unclassified; unclassified; |
| | | | 3.64 | | | | | | | unclassified |
| OTU3191 | Zeller_V4_MiSeq | 1.84 | 0.39; | 3.18E−03 | | | | | 0.252 | Proteobacteria; unclassified; unclassified; |
| | | | 3.29 | | | | | | | unclassified |
| OTU3191 | Brim_V13_454 | −0.08 | −2.70; | 4.18E−03 | | | | | 0.252 | Proteobacteria; unclassified; unclassified; |
| | | | 2.54 | | | | | | | unclassified |

Abbreviations:
LogFC: Log₂Fold Change,
τ2: The (total) amount of heterogeneity among the true effects,
SE: Standard error,
QE: Test statistic for the test of (residual) heterogeneity from the full model,
QEp: p-value associated with QE,
I2: For a random-effects model, I2 estimates (in percent) how much of the total variability in the effect size estimates (which is composed of heterogeneity plus sampling variability) can be attributed to heterogeneity among the true effects,
H2: estimates the ratio of the total amount of variability in the effect size estimates to the amount of sampling variability,
FDR: False Discovery Rate,
RE: Random Effects

TABLE 13
Differentially abundant OTUs in CRA cases as compared to controls identified by the
Random Effects model (QIIME-CR). Taxonomy follows the convention of: phylum, genus,
species. Blanks are given in cases of uncertain classification at a given taxonomic rank.

| | | Log | CILB; | | | SE_T | | | | |
|---|---|---|---|---|---|---|---|---|---|---|
| OTUID | Study | FC | CIUB | p | tau | au 2 | I² | H2 | FDR | Taxonomy |
| OTU1105984 | RE-Model | −2.8 | −4, −1.6 | 6.87E−06 | 0.00E+00 | 1.23 | 0.00 | 1 | 0.002 | Bacteroidetes; Bacteroidaceae; |
| | | | | | | | | | | Bacteroides |
| OTU1105984 | Zack_V4_MiSeq | −3.6 | −5.9, −1.3 | 6.87E−06 | | | | | 0.002 | Bacteroidetes; Bacteroidaceae; |
| | | | | | | | | | | Bacteroides |
| OTU1105984 | Zeller_V4_MiSeq | −2.5 | −4.2, −0.8 | 6.87E−06 | | | | | 0.002 | Bacteroidetes; Bacteroidaceae; |
| | | | | | | | | | | Bacteroides |
| OTU1105984 | Pascual_V13_454 | −2.4 | −5.0, 0.2 | 6.87E−06 | | | | | 0.002 | Bacteroidetes; Bacteroidaceae; |
| | | | | | | | | | | Bacteroides |
| OTU1160847 | RE-Model | 2.6 | 1.4, 3.7 | 1.33E−05 | 0.00E+00 | 1.08 | 0.00 | 1 | 0.002 | Firmicutes; Ruminococcaceae; |
| | | | | | | | | | | Ruminococcus |
| OTU1160847 | Zack_V4_MiSeq | 1.9 | −0.1, 3.9 | 1.33E−05 | | | | | 0.002 | Firmicutes; Ruminococcaceae; |
| | | | | | | | | | | Ruminococcus |
| OTU1160847 | Zeller_V4_MiSeq | 3.1 | 1.4, 4.8 | 1.33E−05 | | | | | 0.002 | Firmicutes; Ruminococcaceae; |
| | | | | | | | | | | Ruminococcus |
| OTU1160847 | Brim_V13_454 | 2.6 | 0.03, 5.2 | 1.33E−05 | | | | | 0.002 | Firmicutes; Ruminococcaceae; |
| | | | | | | | | | | Ruminococcus |
| OTU181871 | RE-Model | 2.3 | 1.2, 3.5 | 3.88E−05 | 0.00E+00 | 1.07 | 0.00 | 1 | 0.005 | Firmicutes; Lachno spiraceae; Dorea |
| OTU181871 | Zack_V4_MiSeq | 1.6 | −0.7, 3.8 | 3.55E−05 | | | | | 0.005 | Firmicutes; Lachno spiraceae; Dorea |
| OTU181871 | Zeller_V4_MiSeq | 2.6 | 1.1, 4.1 | 3.88E−05 | | | | | 0.005 | Firmicutes; Lachno spiraceae; Dorea |
| OTU181874 | Brim_V13_454 | 2.6 | 0.1, 5.1 | 3.88E−05 | | | | | 0.005 | Firmicutes; Lachno spiraceae; Dorea |

Abbreviations:
LogFC: Log₂Fold Change,
τ2: The (total) amount of heterogeneity among the true effects,
SE: Standard error,
QE: Test statistic for the test of (residual) heterogeneity from the full model,
QEp: p-value associated with QE,
I2: For a random-effects model, I2 estimates (in percent) how much of the total variability in the effect size estimates (which is composed of heterogeneity plus sampling variability) can be attributed to heterogeneity among the true effects,
H2: estimates the ratio of the total amount of variability in the effect size estimates to the amount of sampling variability,
FDR: False Discovery
Rate, RE: Random Effects

As described above, in order to identify a composite microbial biomarker for the disease, we developed random forest classifiers for each bioinformatics pipeline. The optimal model was tuned for area under receptor operator characteristic curve (AUROC). For the SS-UP pipeline, microbial markers identified among the 8 studies had an AUROC of 80.4% (Sensitivity: 60.1%, Specificity 84.8%) which was similar to the clinical features-based classifier (AUROC: 79.6%, DeLongs test p=0.76). The SS-UP microbial classifier had improved sensitivity while the clinical classifier had better specificity. The AUROC for the QIIME-CR microbial classifier was 76.6% (Sensitivity: 55.3%, Specificity: 82.9%) (Table 14).

TABLE 14
Random forest classifier characteristics of both pipelines

| | ROC | Sensitivity | Specificity | | ROC | Sensitivity | Specificity | Studies in |
|---|---|---|---|---|---|---|---|---|
| QIIME-CR | Mean | Mean | Mean | SS-UP | Mean | Mean | Mean | the model |
| CRC Vs | | | | | | | | |
| Control | | | | | | | | |
| Clinical | 81.1% | 54.5% | 91.6% | Clinical | 81.1% | 54.5% | 91.6% | [1-3] |
| (n = 156) | | | | (n = 156) | | | | |
| Microbiome | 81.9% | 77.5% | 73.4% | Microbiome | 90.1% | 82.5% | 83.5% | [1-3] |
| subset | | | | subset | | | | |
| (n = 156) | | | | (n = 156) | | | | |
| Microbiome | 75.6% | 55.3% | 82.9% | Microbiome | 80.4% | 60.1% | 84.8% | [1-8] |
| (n = 430) | | | | (n = 430) | | | | |
| Clinical + | 82.4% | 70.6% | 78.5% | Clinical + | 91.8% | 86.2% | 85.4% | [1-3] |
| Microbiome | | | | Microbiome | | | | |
| (n = 156) | | | | (n = 156) | | | | |
| CRA Vs | | | | | | | | |
| Control | | | | | | | | |
| Microbiome | 67.4% | 78.3% | 38.8% | Microbiome | 63.6% | 80.5% | 34.4% | [1 2 5 9] |
| (n = 162) | | | | (n = 162) | | | | |
| CRA Vs | | | | | | | | |
| CRC | | | | | | | | |
| Microbiome | 80.8% | 66.8% | 80.3% | Microbiome | 73.7% | 62.1% | 76.0% | [1 2 5 9] |
| (n = 153) | | | | (n = 153) | | | | |

Abbreviations:
QIIME-CR: QIIME closed reference,
SS-UP: Strain Select UPARSE,
ROC: Receiver Operator Characteristic curve,
CRA: Colorectal adenoma
Mean indicates mean over cross validation folds, Clinical variables included in the Clinical and Clinical +
Microbial classifier were FOBT, Age, gender, BMI, nationality

For both SS-UP and QIIME-CR, 01 Us within Peptostrepococcus amerobius, Porphyrmonas and Dialister ranked high in variable importance. The top features included in the SS-UP microbial classifier were the previously mentioned Parvimonas micra, Dialister pneumosintes ATCC 33048, Peptostreptococcus stomatis DSM 17678, and Bacteroides vulgatus ATCC 84842, while the QIIME-CR approach identified Bulleida moctrei and Eubacterium dolichum as important. OTUs within genus Fusobacterium were also important in discriminating CRC cases from controls.

Using a subset of studies for which both clinical and demographic data was available (n=3 studies, 156 samples) [10-12], the microbial-only classifiers for these studies had AUROC values of 809% for QIIME-CR and 89.6% for SS-UP. As mentioned above, clinical features alone yielded an AUROC of 79.6%, and classifiers including both clinical and microbial features had AUROC values of 82.4% and 91.3% for QIIME-CR and SS-UP, respectively (Table 14).

To determine whether any particular study weighted classifier accuracy we performed an n−1 analysis and evaluated changes in the classifier performance, relative to performance based on the full set of studies (n=8 studies), as each study was excluded one at a time. Excluding Wang_V3_454 [14] reduced the accuracy of the classifier the most (from 80.1 to 75.8%), suggesting that it had important features to contribute. Excluding WuZhu_V3_454 improved the overall accuracy of the SS-UP pipeline (AUROC increased from 80.1 to 83.9%), indicating it contributed ‘noisy’ features that detracted from classifying disease outcome. Similar trends were observed for the QIIME-CR analysis (Table 15).

TABLE 15
Characteristics of the leave one study out and per study random forest classifier

| | | | | | | | | |
|---|---|---|---|---|---|---|---|---|
| Colorectal Cancer vs. | Sample | ROC | Mean | Mean | | | | |
| Control | Size | Mean | sensitivity | specificity | PPV | NPV | mtry | Features |

SS-UP

| | | | | | | | | |
|---|---|---|---|---|---|---|---|---|
| Total microbial cohort | 424 | 80.4% | 60.1% | 84.8% | 77.1% | 71.4% | 55 | 972 |
| Minus Wang_V3_454 | 322 | 75.7% | 54.5% | 83.2% | 73.7% | 68.0% | 65 | 1049 |
| Minus Chen_V13_454 | 382 | 79.4% | 60.3% | 84.6% | 76.9% | 71.6% | 48 | 993 |
| Minus WuZhu_V3_454 | 393 | 83.9% | 65.5% | 86.0% | 80.1% | 74.3% | 56 | 1001 |
| Minus Weir_V4_454 | 411 | 80.6% | 61.4% | 83.5% | 76.0% | 71.7% | 64 | 995 |
| Minus | 333 | 78.5% | 59.0% | 83.1% | 75.0% | 70.2% | 49 | 776 |
| Zeller_V4_MiS | | | | | | | | |
| Minus Zack_V4_MiSeq | 364 | 78.6% | 59.2% | 85.2% | 76.9% | 71.6% | 55 | 926 |
| Minus | 406 | 81.6% | 62.7% | 85.0% | 77.9% | 72.9% | 48 | 988 |
| Pascual_V13_45 | | | | | | | | |
| Minus | 357 | 83.1% | 64.4% | 85.1% | 78.8% | 73.5% | 63 | 990 |
| Flemer_V34_MiSe | | | | | | | | |
| Only Wang_V3_454 | 102 | 89.6% | 81.7% | 89.6% | 86.6% | 85.7% | 43 | 292 |
| Only Chen_V13_454 | 42 | 80.5% | 54.0% | 73.6% | 65.1% | 63.8% | 10 | 347 |
| Only WuZhu_V3_454 | 31 | 84.7% | 9.2% | 76.7% | 22.2% | 53.9% | 29 | 350 |
| Only Weir_V4_454 | 13 | 100.0% | 20.0% | 85.7% | 54.5% | 55.6% | 7 | 153 |
| Only Zeller_V4_MiSeq | 91 | 89.9% | 70.7% | 86.8% | 81.5% | 78.3% | 66 | 1073 |
| Only Zack_V4_MiSeq | 60 | 96.5% | 88.7% | 85.3% | 85.8% | 88.3% | 92 | 934 |
| Only | 18 | 100% | 46.7% | 80.0% | 70.0% | 60.0% | 11 | 460 |
| Pascual_V13_45 | | | | | | | | |
| Only | 67 | 77.6 | 76.7% | 60.0% | 69.0% | 68.9% | 41 | 715 |
| Flemer_V34_MiSe | | | | | | | | |

QIIME-CR

| | | | | | | | | |
|---|---|---|---|---|---|---|---|---|
| Total microbial cohort | 424 | 75.6% | 55.3% | 82.9% | 73.4% | 68.5% | 194 | 4160 |
| Minus Wang_V3_454 | 322 | 70.7% | 81.7% | 89.6% | 86.6% | 85.7% | 102 | 4542 |
| Minus Chen_V13_454 | 382 | 74.9% | 54.0% | 73.6% | 65.1% | 63.8% | 130 | 4212 |
| Minus WuZhu_V3_454 | 393 | 79.3% | 60.3% | 82.0% | 74.3% | 70.6% | 130 | 4206 |
| Minus Weir_V4_454 | 411 | 76.3% | 55.8% | 82.3% | 72.9% | 68.6% | 131 | 4271 |
| Minus | 333 | 73.9% | 54.9% | 82.0% | 72.4% | 67.9% | 114 | 3233 |
| Zeller_V4_MiS | | | | | | | | |
| Minus Zack_V4_MiSeq | 364 | 73.7% | 58.7% | 82.9% | 74.4% | 70.4% | 128 | 4068 |
| Minus | 406 | 76.9% | 56.1% | 83.2% | 74.1% | 69.0% | 115 | 4245 |
| Pascual_V13_45 | | | | | | | | |
| Minus | 357 | 78.6% | 59.1% | 85.4% | 75.2% | 71.1% | 128 | 4312 |
| Flemer_V34_MiSe | | | | | | | | |
| Only Wang_V3_454 | 102 | 84.1% | 70.0% | 85.4% | 79.7% | 77.6% | 51 | 818 |
| Only Chen_V13_454 | 42 | 77.3% | 52.0% | 74.5% | 65.0% | 63.1% | 130 | 1867 |
| Only WuZhu_V3_454 | 31 | 86.0% | 1.5% | 82.2% | 5.9% | 53.6% | 85 | 2355 |
| Only Weir_V4_454 | 13 | 100% | 43.3% | 68.6% | 54.2% | 58.5% | 18 | 1161 |
| Only Zeller_V4_MiSeq | 91 | 84.7% | 67.3% | 86.4% | 80.2% | 76.3% | 176 | 4915 |
| Only Zack_V4_MiSeq | 60 | 92.4% | 87.3% | 85.3% | 85.6% | 87.1% | 185 | 3556 |
| Only | 18 | 100.0% | 28.9% | 57.8% | 40.6% | 44.8% | 19 | 2673 |
| Pascual_V13_45 | | | | | | | | |
| Only | 67 | 71.50% | 43.3% | 81.1% | 65.0% | 63.8% | 156 | 3321 |
| Flemer_V34_MiSe | | | | | | | | |

Abbreviations:
QIIME-CR; QIIME closed reference,
SS-UP: Strain Select UPARSE,
ROC: Receiver Operator Characteristic curve,
PPV—Positive Predictive Value,
NPV—Negative Predictive Value,
mtry—tuning parameter to determine number of features subsampled at each node in random forest analysis,
Features: total number of microbial features used in the random forest analysis
Mean indicates mean over cross validation folds

We constructed an RF model for each study individually and observed that features identified within a single study with homogenously processed samples frequently had a better ROC, but the sensitivity of the individual study models was often lower than that obtained for the combined classifier (Table 15).

To test the generalizability of the classifier, we observed the degree to which an n−1 microbial classifier was able to predict disease outcome in the study that was left out. For example, we considered the (n-Chen_V13_454 cohort) as the training set and the Chen_V13_454 as the validation set and determined how well disease outcome in the Chen et al cohort was predicted by microbial features from the rest of the studies. We observed that microbial features from the rest of the cohort correctly predicted 36/42 samples (AUROC: 80.5%, accuracy: 84.6%) in Chen_V13_454. The predictive value varied among studies (Table 16).

TABLE 16
Prediction accuracy of the n study -1 cohort on the excluded study (SS-UP)

| | | Prediction | Correctly | Percent |
|---|---|---|---|---|
| Training Set | Validation set | AUROC | predicted | prediction |
| Minus Wang_V3_454 | Only Wang_V3_454 | 73.6% | 49/91 | 53.8% |
| Minus Chen_V13_454 | Only Chen_V13_454 | 80.5% | 36/42 | 85.7% |
| Minus WuZhu_V3_454 | Only WuZhu_V3_454 | 57.6% | 16/31 | 51.6% |
| Minus Weir_V4_454 | Only Weir_V4_454 | 76.2% | 8/13 | 61.5% |
| Minus Zeller_V4_MiSeq | Only Zeller_V4_MiSeq | 82.5% | 59/81 | 72.8% |
| Minus Zackular_V4_MiSeq | Only | 74.2% | 41/60 | 68.3% |
| | Zackular_V4_MiSeq | | | |
| Minus Pascual_V13_454 | Only Pascual_V13_454 | 62.3% | 48/66 | 72.7% |
| Minus Flemer_V34_MiSeq | Only | 63.5% | 11/17 | 64.7% |
| | Flemer_V34_MiSeq | | | |

Abbreviation: SS-UP: Strain Select UPARSE, AUROC: Area Under Receiver Operating Characteristic curve

TABLE 17
Top 25 OTUs across analyses (SS-UPJ

| | | Consistently | Important in |
|---|---|---|---|
| | Differentially | variable across | CRC |
| Microbial marker | abundant | studies | classification |
| Parvimonas micro ATCC 32770 | ↑ ✓ | ↑ ✓ | ✓ |
| Proteobacteria OTU 3191 | ↑ ✓ | ↑ ✓ | ✓ |
| Fusobacterium sp. OTU 2790 | ↑ ✓ | ↑ ✓ | ✓ |
| Dialister sp. OTU 2589 | ↑ ✓ | ↑ ✓ | ✓ |
| Enterococcus sp. OTU 910 | ↑ ✓ | ↑ ✓ | ✓ |
| Akkermansia muciniphila OTU 3364 | ↑ ✓ | ↑ ✓ | |
| Parvimonas sp OTU 1169 | ↑ ✓ | ✓ | |
| Peptostreptococcus stomatis DSM 17678 | ↑ ✓ | | ✓ |
| Peptostreptococcus anaerobius OTU2049 | ↑ ✓ | | ✓ |
| Dialister pneumosintes ATCC 33048 | ↑ ✓ | | ✓ |
| Clostridium spiroforme DSM 1552 | ↑ ✓ | | ✓ |
| Actinobacteria OTU 295 | ↑ ✓ | | ✓ |
| Porphyromonas asaccharolytica DSM | ↑ ✓ | | ✓ |
| 20707 | | | |
| Porphyromonas OTU 569 | ↑ ✓ | | ✓ |
| Lactobacillus OTU 969 | ↑ ✓ | | ✓ |
| Streptococcus anginosus OTU 1044 | | ↑ ✓ | ✓ |
| Firmicutes OTU 1255 | | ↑ ✓ | ✓ |
| Lachnospira OTU 1926 | | ↑ ✓ | ✓ |
| Oscillospora OTU 2405 | | ↑ ✓ | ✓ |
| Eubacterium dolichum OTU 2691 | | ↑ ✓ | ✓ |
| Bacteroides caccae OTU467 | | ↑ ✓ | ✓ |

Upward arrows indicate taxa were elevated m CRC cases as compared to controls. Downward arrows indicate that taxa were elevated in controls relative to cases
Abbreviation: SS-UP—Strain Select - UPARSE
Differentially abundant: Selected by DESeq2 Log2Fold change >1.5, <−1.5, FDR p < 0.05 Consistently variable across studies: Have an adjusted Random Effects Log2Fold change of >1 or <−1 or FDR adjusted RE-model p of <0.5. Important in Classification: >10% importance in microbial feature RF classifier. OTUs were picked that satisfied at least two of the three criteria mentioned above.

The CRA versus control SS-UP classifier, which combined microbial taxa from four studies, had lower accuracy than the CRC classifier (AUROC: 63.6%) but good sensitivity (80.5%) and low specificity (34.4%). The QIIME-GR CRA microbial classifier had similar metrics (AUROC: 67.4%, sensitivity: 78.3%, specificity: 38.8%). We also attempted to classify CRA versus CRC samples and obtained moderately good classification accuracy (SS-UP AUROC: 73.7%, QIIME AUROC: 80.7%).

Finally, we combined microbial markers from the analyses above for the CRC vs control comparison to identify a common set that was differentially abundant, consistent across studies, and important in classification. This list of 25 microbial OTUs from the SS-UP pipeline is highlighted in the Table 17.

### Discussion

Most previously reported microbiome meta-analyses have employed a closed-referenced strategy for processing 16S data [20, 22, 41], In the present study, we assembled a diverse collection of microbiome studies and evaluated both the closed-reference approach and an alternate method of combining open-reference OTU picking and reclassifying de nova OTUs against a reference database. By repositioning raw sequencing data from multiple fecal microbiome studies and analyzing it in a uniform manner, we identified microbial markers which were consistently enriched or depleted in CRC. Importantly, we identified novel and previously unreported strains associated with CRC and CRA without the use of shotgun metagenomic sequencing.

Despite the heterogeneity associated with each of the original microbiome studies, the RF classifiers we built were comparable to results reported by Zeller et al et al [10] (shotgun metagenomic classifier of 22 taxa with an AUROC of 84%), Zackular et al (six taxa with an AUROC of 79%), and Baxter et al (42) (microbial markers classifying colonic lesions with an AUROC of 84.7%). [42] The SS-UP-based classifiers consistently yielded greater sensitivity and specificity, while also producing fewer predictors (i.e., OTUs) and tuning variables (mtry) than the QIIME-CR approach. The SS-UP microbial classifier had an accuracy of 80.1%, and the exclusion of the Wu_V3_454 study (n=39) resulted in a similar AUROC to that of Baxter et al [42]. The results obtained from the SS-UP pipeline for models evaluating microbial features (AUROC 89.6%) or microbial features plus FOBT results, age, gender, and B1141 (AUROC 91.8%) from a subset of studies [10-12] were comparable to the combined metagenomic and FOBT classifiers reported by Zeller et al (AUROC of 87%) and Zackular et al (AUROC of 93.6%). Similarly, Baxter et al reported a combined classifier based on microbial markers and the fecal immunochemical test (FIT), an alternative screening method to FOBT, to have an AUROC of 95.2%. [42] Therefore this is the first report of a CRC stool classifier to achieve an AUROC >84% while simultaneously incorporating variation across 8 cohorts and multiple laboratory protocols.

Notably, the results of our leave-one-out analysis suggest that the SS-UP classifier was not drastically affected by features unique to any particular study. This demonstrates the stability of microbial markers as a reliable classification tool for CRC. To further establish the generalizability of the SS-UT microbial classifier, when the study that was excluded in the leave one out analysis was treated as an external validation cohort, the average prediction AUROC was 71.3% (Table 16).

We report an OTU bearing a high degree of similarity to Parvimonas micra ATCC 33270 to be consistently elevated in CRC cases, as well as ranked highly in the microbial and combined clinical-microbial classifier models. As suggested previously, [43] markers of periodontal disease, such as Peptostreptococcus, Porphyromonas and OTUs within Diallister sp, demonstrated high classification power for both pipelines. (Tables 6-7) Oral pathogens have been described in association with CRC and multiple mechanisms have been postulated to explain this relationship. [41, 44] The SS-UP pipeline also identified the enrichment of strains within the genus Blautia (e.g., Blautia luti DSM14534 and Blautia obeum ATCC 29174) which have been previously implicated in CRC cases [26, 45] and the depletion of potentially beneficial microbes, such as dietary carcinogen-transforming Eubacterium hallii [46] (strain DSM 3353) and butyrate-producing Faecalibacterium cf prausnitzii [12 27] (strain KLE1255) (Table 6).

Both the SS-UP and QIIME-CR pipelines found Fusobacterium sp., one of the most commonly reported bacterial taxa in CRC studies, to be enriched in CRC cases relative to controls. It was significantly enriched in CRC cases in our differential abundance analyses and ranked high in importance in the combined (clinical+microbial) RF model, both of which were pooled analyses and had the potential to be weighted by two large MiSeq studies. In a per-study analysis, we identified a Fusobacterium OTU with a significantly high log₂ fold change in those MiSeq studies which targeted the V3 and/or V4 regions, but its relative abundance and distribution was far more variable when compared across all studies. This suggests that the detection and reporting of Fusobacterium sp. in conjunction with CRC may be dependent on the 16S target region (e.g., V3/V4 amplicons) and/or sequencing platform utilized. Although Fusobacterium sp. was enriched in CRC samples, it was not found to be differentially abundant in CRA samples for either pipeline by univariate analysis, REM, or RF classification models, indicating that it may be a marker of late(r) stage disease.

CRA or pre-cancerous lesions were not sufficiently distinguished from controls by microbial markers by either bioinformatics pipeline. Although a previously published study reported a combination of five OTUs with an AUROC of 83.9% to differentiate adenoma from controls, another study utilizing a different cohort and twenty microbial taxa resulted in an ROC of 67.3% in the identification of CRA. The combination of microbial and clinical markers appears to provide greater diagnostic utility for CRA than microbial markers alone. Notably, the combination of FIT testing and phylum-level microbial abundances has been reported to have an AUROC of 76.7% to classify CRA. [30] Compared to previously published studies, the sensitivity of our microbial marker-only SS-UP classifier was relatively high (75.5%) and could be used to complement an FORT or FIT tests, which have greater specificity [24, 30].

Our CRA vs CRC classification yielded a better AUROC than the healthy vs CRA comparison in our analysis, or those from other studies. [11, 42] Thus, changes in microbial composition appear to be most apparent in the adenoma-carcinoma transition but not necessarily at polyp initiation. Differential abundance analysis identified some of the same OTUs within Succinovibrio and Clostridia in the comparison of both CRA and CRC cases to controls, and it is possible that these may serve as “driver” species in cancer progression. Whether driver or passenger, these observational studies confirm that microbial dysbiosis is a characteristic feature of CRC and presents a promising target for detection and intervention.

Despite best efforts, there were certain limitations. Information regarding cancer stage, tumor location, FOIST results, and patient demographics, including age, gender, and BMI was available for only three of the nine studies analyzed. Likewise, information regarding adenoma growth patterns (e.g., tubular or villous) and cancerous capacity (i.e., neoplastic or hyperplastic) was limited. Statistically, differential abundance analyses are sensitive to sparse microbial OTU data (which is a characteristic of microbial taxa distribution) and variation with respect to depth of coverage. We attempted to control for potentially artefactual results by adjusting for confounders and correcting for multiple comparisons.

Despite these limitations, our study assembled and uniformly analyzed a diverse set of fecal microbiome CRC data sets, identified key taxa that were consistently elevated in CRC cases, and determined a composite set of 165 rRNA gene-based fecal microbial biomarkers for CRC detection, representing a key step forward in the search for a sensitive, specific, and non-invasive diagnostic for CRC.

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

1. Cancer Facts and Figures 2016: American Cancer Society, 2016.
2. Parkin D M, Olsen A H, Sasieni P. The potential for prevention of colorectal cancer in the UK. European journal of cancer prevention: the official journal of the European Cancer Prevention Organisation (ECP) 2009; 18(3): 179-90 doi: 10.1097/CEJ.0b013e32830c8d83 [published Online First: Epub Date]
Giacosa A, Franceschi S, La Vecchia C, Favero A, Andreatta R. Energy intake, overweight, physical exercise and colorectal cancer risk. European journal of cancer prevention: the official journal of the European Cancer Prevention Organisation (ECP) 1999; 8 Suppl 1:S53-60.
4. Shah M S, Fogelman D R, Raghav K P, et al. Joint prognostic effect of obesity and chronic systemic inflammation in patients with metastatic colorectal cancer. Cancer 2015; 121(17):2968-75 doi: 10.1002/cncr. 29440 [published Online First: Epub Date]
5. Vital signs: Colorectal cancer screening, incidence, and mortality United States, 2002-2010. MMWR. Morbidity and mortality weekly report 2011; 60(26):884-9
6. Samadder N J, Curtin K, Tuohy T M, et al. Characteristics of missed or interval colorectal cancer and patient survival: a population-based study. Gastroenterology 2014; 146(4):950-60 doi: 10.1053/j.gastro.2014.01.013 [published Online First: Epub Date]
7. Hundt S, Haug U, Brenner H. Comparative evaluation of immunochemical fecal occult blood tests for colorectal adenoma detection. Ann Intern Med 2009; 150(3):162-9
8. Imperiale T F, Ransohoff D F, Itzkowitz S H, et al. Multitarget Stool DNA Testing for Colorectal-Cancer Screening. New England Journal of Medicine 2014; 370(14):1287-97 doi:doi: 10.105 6/NEJMoal 311194 [published Online First: Epub Date]
9. Chustecka Z, High Price Tag for Cologuard Confirmed, but Test Is Welcomed. Medscape Medical News 2014. www.medscape.com/viewarticle/835506.
10. Zeller G, Tap J, Voigt A Y, et al. Potential of fecal microbiota for early-stage detection of colorectal cancer. Molecular systems biology 2014; 10:766 doi: 10.15252/msb.20145645 [published Online First: Epub Date]
11. Zackular J P, Rogers M A, Ruffin M Tt, Schloss P D. The human gut microbiome as a screening tool for colorectal cancer. Cancer prevention research (Philadelphia, Pa.) 2014; 7(11):1112-21 doi: 10.1158/1940-6207.capr-14-01.29[published Online First: Epub Date]
12. Wu N, Yang X, Zhang R, et al. Dysbiosis signature of fecal microbiota in colorectal cancer patients. Microbial ecology 2013; 66(2):462-70 doi: 10.1007/s00248-013-0245-9[published Online First: Epub Date].
13. Weir T L, Manter D K, Sheflin A M, Barnett B A, Heuberger A L, Ryan E P. Stool microbiome and metabolome differences between colorectal cancer patients and healthy adults. PloS one 2013; 8(8):e70803 doi: 10.1371/journal.pone.0070803 [published Online First: Epub Date].
14. Wang T, Cai G, Qiu Y, et al. Structural segregation of gut microbiota between colorectal cancer patients and healthy volunteers. The ISMS journal 2012; 6(2):320-9 doi: 10.1038/ismej.2011.109 [published Online First: Epub Date].
15. Sobhani I, Tap J, Roudot-Thoraval F, et al. Microbial dysbiosis in colorectal cancer (CRC) patients. PloS one 2011; 6(1):e16393 doi: 10.1371.journal.pone.0016393 [published Online First: Epub Date].
16. Marchesi J R, Dutilh B E, Hall N, et al. Towards the human colorectal cancer microbiome. PloS one 2011; 6(5):e20447 doi: 10.137/journal.pone.0020447[published Online First: Epub Date].
17. Kostic A D, Gevers D, Pedamallu C S, et al. Genomic analysis identifies association of Fusobacterium with colorectal carcinoma. Genome research 2012; 22(2):292-98 doi: 10.1101/gr.126573.111 [published Online First: Epub Date],
18. Dingemanse C, Belzer C, van Hijum S A, et al. Akkermansia muciniphila and Helicobacter typhionius modulate intestinal tumor development in mice. Carcinogenesis 2015; 36(11):1388-96 doi: 10.1093/carcin/bgv120[published Online First: Epub Date].
19. Castellarin M, Warren R L, Freeman J D, et al. Fusobacterium nucleatum infection is prevalent in human colorectal carcinoma. Genome research 2012; 22(2):299-306 doi: 10.1101/gr.126516.111 [published Online First: Epub Date],
20. Lozupone C A, Stombaugh J, Gonzalez A, et al. Meta-analyses of studies of the human microbiota. Genome research 2013; 23(10):1704-14 doi: 10.1101/gr.151803.112[published Online First: Epub Date],
21. Adams R, Bateman A, Bik H, Meadow J. Microbiota of the indoor environment: a meta-analysis. Microbiome 2015; 3(1):49.
22. Walters W A, Xu Z, Knight R. Meta-analyses of human gut microbes associated with obesity and IBD. FEBS letters 2014; 588(22):4223-33 doi: 10.1016/j.febstet.201.4.09.039[published Online First: Epub Date].
23. Hewitson P, Glasziou P, Watson E, Towler B, Irwig L. Cochrane systematic review of colorectal cancer screening using the fecal occult blood test (hemoccult): an update. The American journal of gastroenterology 2008; 103(6).1541-9 doi: 10.111/j.1572-0241.2008.01875. x[published Online First: Epub Date].
24. Wong C K, Fedorak R N, Prosser C I, Stewart M E, van Zanten S V, Sadowski D C. The sensitivity and specificity of guaiac and immunochemical fecal occult blood tests for the detection of advanced colonic adenomas and cancer. International journal of colorectal disease 2012; 27(12):1657-64 doi: 10.1007/s00384-012-1518-3[published Online First: Epub Date].
25. Stroup D F, Berlin J A, Morton S C, et al. Meta-analysis of observational studies in epidemiology: A proposal for reporting. Jama 2000; 283(15):2008-12 doi: 10.1001/jama.283.15.2008[published Online First: Epub Date].
26. Chen W, Liu F, Ling Z, Tong X, Xiang C. Human intestinal lumen and mucosa-associated microbiota in patients with colorectal cancer. PloS one 2012; 7(6):e39743 doi: 10.1371/journal.pone.0039743 [published Online First: Epub Date].
27. Mira-Pascual L, Cabrera-Rubio R, Ocon S, et al. Microbial mucosal colonic shifts associated with the development of colorectal cancer reveal the presence of different bacterial and archaeal biomarkers. J Gastroenterol 2015; 50(2):167-79 doi: 10.1007/s00535-014-0963-x[published Online First: Epub Date].
28. Flemer B, Lynch D B, Brown J M, et al. Tumour-associated and non-tumour-associated microbiota in colorectal cancer. Gut 2016 doi: 10.1136/gutjnl-2015-309595[published Online First: Epub Date],
29. Brim H, Yooseph S, Zoetendal E G, et al. Microbiome analysis of stool samples from African Americans with colon polyps. PloS one 2013; 8(12):e81352 doi: 10.1 371/journal.pone.0081352[published Online First: Epub Date].
30. Goedert J J, Gong Y, Hua X, et al. Fecal Microbiota Characteristics of Patients with Colorectal Adenoma Detected by Screening: A Population-based Study. EBioMedicine 2015; 2(6):597-603 doi: 10.1016/j.ebiom.2015.04.010[published Online First: Epub Date].
31. Ahn J, Sinha R, Pei Z, et al. Human gut microbiome and risk for colorectal cancer. Journal of the National Cancer Institute 2013; 105(24).1907-11 doi: 10.1093/jnci/djt300[published Online First: Epub Date].
32. Chen H M, Yu Y N, Wang J L, et al. Decreased dietary fiber intake and structural alteration of gut microbiota in patients with advanced colorectal adenoma. The American journal of clinical nutrition 2013; 97(5):1044-52 doi: 10.3 945/ajcn. 112.046607 [published Online First: Epub Date]|.
33. Caporaso J G, Kuczynski J, Stombaugh J, et al. Q11ME allows analysis of high-throughput community sequencing data. Nature methods 2010; 7(5):335-6 doi: 10.1038/nmeth.f.303 [published Online First: Epub Date]|.
34. Edgar R C. UPARSE: highly accurate OTU sequences from microbial amplicon reads. Nat Meth 2013, 10(10): 996-98 doi: 10.1038/nmeth. 2604 www.nature.com/nmeth/journal/v10/n10/abs/nmeth. 2604. html#supplementary-information[published Online First: Epub Date]|.
35. McMurdie P J, Holmes S. Waste Not, Want Not: Why Rarefying Microbiome Data Is Inadmissible. PLoS Comput Biol 2014, 10(4):e1003531 doi: 10.1371/journal.pcbi.1003531[published Online First: Epub Date]|.
36. McMurdie P J, Holmes S. phyloseq: An R Package for Reproducible Interactive Analysis and Graphics of Microbiome Census Data. PloS one 2013; 8(4):e61217 doi: 10.1371/journal.pone.0061217[published Online First: Epub Date]|.
37. Oksanen F G B, Roeland Kindt, Pierre Legendre, Peter R. Minchin, R. B. O'Hara, Gavin L. Simpson, Peter, Solymos MHHSaHW. vegan: Community Ecology Package. 2015
38. Viechtbauer W. Conducting Meta-Analyses in R with the metafor Package. 2010 2010; 36(3):48 doi: 10,18637/jss.v036.i03 [published Online First: Epub Date]|.
39. Kuhn M. Building Predictive Models in R Using the caret Package. Journal of Statistical Software 2008; 28(5):1-26 doi:citeulike-article-id:6573927[published Online First: Epub Date]|.
40. Wiener ALaM, Classification and Regression by randomForest. R News 2002; 2(3):18-22
41. Adams R I, Bateman A C, Bik H M, Meadow J F. Microbiota of the indoor environment: a meta-analysis. Microbiome 2015; 3:49 doi: 10.1186/s40168-015-0108-3[published Online First: Epub Date]|.
42. Baxter N T, Ruffin M Tt, Rogers M A, Schloss P D. Microbiota-based model improves the sensitivity of fecal immunochemical test for detecting colonic lesions. Genome Med 2016; 8(1):37 doi: 10.1186/s13073-016-0290-3[published Online First: Epub Date]|.

What is claimed is:

1. A method for diagnosing colorectal cancer (CRC) or colorectal adenoma (CRA) in a subject, comprising:
obtaining an intestinal sample from the subject;
processing the intestinal sample to obtain 16S rRNA gene sequence data;
detecting the level of one or more microorganisms and or operational taxonomic units (OTUs) in the intestinal sample comprising analyzing the 16S rRNA gene sequence data; and
diagnosing the subject as having CRC or CRA or is at the risk of developing CRC or CRA when the level of two or more microorganisms and or OTUs in the intestinal sample is increased relative to a control sample;
wherein the two or more microorganisms and or OTUs are selected from the group of microorganisms and or OTUs listed in Table 1.

2. The method of claim 1, wherein the two or more microorganisms and/or OTUs are selected from the group consisting of OTU Identifiers: OTU1167, OTU3191, OTU2573, OTU1044, OTU567 and OTU1873.

3. The method of claim 1, wherein the two or more microorganisms and or OTUs are selected from the group consisting of OTU Identifiers: OTU1167, OTU2790, OTU3191 and OTU1044.

4. The method of claim 1, wherein the step of analyzing the 16S rRNA gene sequence data comprises extracting microbial polynucleotides from the intestinal sample, sequencing the 16S rRNA polynucleotides extracted from the intestinal sample, aligning 16S rRNA sequences from the intestinal sample of the subject against reference sequences in the StrainSelect database and performing a de novo clustering using SS-UP.

5. The method of claim 4, wherein the step of analyzing the 16S rRNA gene sequence data using SS-UP provides a strain-level resolution of microorganisms and/or OTUs.

6. The method of claim 4, wherein the step of analyzing the 16S rRNA gene sequence data using SS-UP provides an area under receiver operator characteristic (AUROC) curve of at least about 80%.

7. The method of claim 4, wherein the step of analyzing the 16S rRNA gene sequence data using SS-UP provides a strain-level resolution of OTUs compared to a species-level resolution provided by QIIME-CR.

8. The method of claim 1, wherein the step of detecting the level of one or more microorganisms and/or OTUs comprises performing an assay which comprises hybridizing a plurality of oligonucleotides to the OTU polynucleotides sequences in Table 1.

9. The method of claim 8, wherein the plurality of oligonucleotides comprises oligonucleotides which selectively hybridize to at least one of SEQ ID NOS:1-660.

10. The method of claim 8, wherein the one or more microorganisms and/or OTUs are selected from the group consisting of: OTU1167 (SEQ ID NOS:641-647), OTU3191 (SEQ ID NOS:291-513), OTU1873 (648-654), OTU2573 (SEQ ID NOS:8-14), OTU567 (SEQ ID NOS:655-660), and OTU1044 (SEQ ID NOS:15-25).

11. The method of claim 8, wherein the one or more microorganisms and/or OTUs are selected from the group consisting of: OTU1167 (SEQ ID NOS:641-647), OTU3191 (SEQ ID NOS:291-513), OTU2790 (SEQ ID NOS:191-248), and OTU1044 (SEQ ID NOS:15-25).

12. The method of claim 1, wherein the subject is diagnosed as having CRC or CRA or is at the risk of developing CRC or CRA when the level of the two or more microorganisms and/or OTUs in the intestinal sample is increased by at least about 5%, relative to the control sample.

13. The method of claim 1, wherein the subject is diagnosed as having CRC or CRA or is at the risk of developing CRC or CRA when the level of one or more microorganisms and/or OTUs in the intestinal sample is increased by at least about 1.2 fold on the log₂ fold-change scale, relative to the control sample.

14. The method of claim 1, wherein the subject is diagnosed as having CRC or CRA or is at the risk of developing CRC or CRA when the level of one or more microorganisms and/or OTUs in the intestinal sample is increased by at least about 2-fold relative to the control sample.

15. The method of claim 1, wherein the control sample is an intestinal sample collected from at least 5 healthy individuals.

16. The method of claim 1, wherein the intestinal sample is a stool sample.

17. The method of claim 1, wherein the method comprises diagnosing the subject as having CRC or is at the risk of developing CRC when the level of the two or more microorganisms in the stool sample is increased relative to a control sample.

18. A diagnostic tool for diagnosing CRC or CRA in a subject, comprising a plurality of oligonucleotides complementary to at least one OTU for each of OTU1167 (SEQ ID NOS:641-647), OTU3191 (SEQ ID NOS:291-513), OTU1873 (648-654), OTU2573 (SEQ ID NOS:8-14), OTU567 (SEQ ID NOS:655-660), and OTU1044 (SEQ ID NOS:15-25).


SEQUENCE LISTING (666 sequences)

SEQ ID NO: 1 - DNA, 252 bp, Clostridium spiroforme
tacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaatagg

SEQ ID NO: 2 - DNA, 252 bp, Clostridium spiroforme
tacgtaggtggcgagcgttatccggaattattgggcgtaaagagggagcaggcggcggcagaggtctatggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaatagg

SEQ ID NO: 3 - DNA, 161 bp, Clostridium spiroforme
tagggaattttcggcaatgggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgcc

SEQ ID NO: 4 - DNA, 160 bp, Clostridium spiroforme
tagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtg

SEQ ID NO: 5 - DNA, 464 bp, Clostridium spiroforme
cctacgggtggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtc

SEQ ID NO: 6 - DNA, 203 bp, Clostridium spiroforme
ccacactgggactgagacacggcccagactcctacgggaggcagcagtagggaattttcggcaatgggggaaccctgaccgagcaacgccgcgtgaaggaagaaggaattcgttctgtaaacttctgttataaaggaagaacggcggatatagggaatgatatccgagtgacggtactttatgagaaagccacggctaactac

SEQ ID NO: 7 - DNA, 230 bp, Clostridium spiroforme
ccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgcc

SEQ ID NO: 8 - DNA, 252 bp, Dialister pneumosintes
tacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagtgtagcggtgaaatgcgtagagattaggaagaacaccggtggcgaaggcgactttctagacgaaaactgacgctgaggcgcgaaagcgtggggagcaaacagg

SEQ ID NO: 9 - DNA, 252 bp, Dialister pneumosintes; identical to SEQ ID NO: 8

SEQ ID NO: 10 - DNA, 160 bp, Dialister pneumosintes
tggggaatcttccgcaatggacgaaagtctgacggagcaacgccgcgtgaacgaagaaggtcttcggattgtaaagttctgtgattcgggacgaaagggtttgtggtgaataatcatagacattgacggtaccgaaaagcaagccacggctaactacgtg

SEQ ID NO: 11 - DNA, 161 bp, Dialister pneumosintes
tggggaatcttccgcaatggacgaaagtctgacggagcaacgccgcgtgaacgaagaaggtcttcggattgtaaagttctgtgattcgggacgaaagggtttgtggtgaataatcatagacattgacggtaccgaaaaagcaagccacggctaactacgtg

SEQ ID NO: 12 - DNA, 465 bp, Dialister pneumosintes
cctacgggaggcagcagtggggaatcttccgcaatggacgaaagtctgacggagcaacgccgcgtgaacgaagaaggtcttcggattgtaaagttctgtgattcgggacgaaagggtttgtggtgaataatcatagacattgacggtaccgaaaaagcaagccacggctaactacgtgccagcagccgcggtaatacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagtgtagcggtgaaatgcgtagagattaggaagaacaccggtggcgaaggcgactttctagacgaaaactgacgctgaggcgcgaaagcgtggggagcaaacaggattagataccctggtagtc

SEQ ID NO: 13 - DNA, 502 bp, Dialister pneumosintes
ctggcggcgtgcttaacacatgcaagtcgaacggaaagagatgaaagagcttgctcttttattaatttcagtggcaaacgggtgagtaacacgtaaacaacctgccttaaggatgggataacagacggaaacgactgctaataccgaatacgttctaagcatcgcatggtgcatagaagaaagggtggcctctacaagaaagctatcgccttaagagggtttgcgactgattaggtagttggtgaggtaacggctcaccaagccgacgatcagtagccggtctgagaggatgaacggccacactggaactgagacacggtccagactcctacgggaggcagcagtggggaatcttccgcaatggacgaaagtctgacggagcaacgccgcgtgaacgaagaaggtcttcggattgtaaagttctgtgattcgggacgaaagggtttgtggtgaataatcatagacattgacggtaccgaaaaagcaagccacggctaactac

SEQ ID NO: 14 - DNA, 421 bp, Dialister pneumosintes
gtaacacgtaaacaacctgcttaaggatgggataacagacgaaacgactgctaataccgaatacgttctaagcatcgcatggtgcatagaagaaagggtggcctctacaagaaagctatcgccttaagaggggtttgcgactgattaggtagttggtgaggtaacggctcaccaagccgacgatcagtagccggtctgagaggatgaacggccacactggaactgagacacggtccagactcctacgggaggcagcagtggggaatcttccgcaatggacgaaagtctgacggagcaacgccgcgtgaacgaagaaggtcttcggattgtaaagttctgtgattcgggacgaaagggtttgtggtgaataatcatagacattgacggtaccgaaaaagcaagccacggctaactacgtgcc

SEQ ID NO: 15 - DNA, 472 bp, Streptococcus anginosus
agagtttgatcatggctcaggacgaacgctggcggcgtgcctaatacatgcaagtaggacgcacagtttataccgtagcttgctacaccatagactgtgagttgcgaacgggtgagtaacgcgtaggtaacctgcctattagagggggataactattggaaacgatagctaataccgcataacagtatgtaacacatgttagatgcttgaaagatgcaattgcatcgctagtagatggacctgcgttgtattagctagtaggtagggtaatggcctacctaggcgacgatacatagccgacctgagagggtgatcggccacactgggactgagacacggcccagactcctacgggaggcagcagtagggaatcttcggcaagtgggggaaccctgaccgagcaacgccgcgtgagtgaagtaaggttttcggatcgtaaagctctgttgttaaggaagaacgagtgtgagaa

SEQ ID NO: 16 - DNA, 229 bp, Streptococcus anginosus
agagtttgatcatggctcaggacgaacgctggcggcgtgcctaatacatgcaagtaggacgcacagtttataccgtagcttgctacaccatagactgtgagttgcgaacgggtgagtaacgcgtaggtaacctgcctattagagggggataactattggaaacgatagctaataccgcataacagtatgtaacacatgttagatgcttgaaagatgcaattgcatcgct

SEQ ID NO: 17 - DNA, 252 bp, Streptococcus anginosus
tacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacagg

SEQ ID NO: 18 - DNA, 251 bp, Streptococcus anginosus
tacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacag

SEQ ID NO: 19 - DNA, 120 bp, Streptococcus anginosus
tagggaatcttcggcaatggggggaaccctgaccgagcaacgccgcgtgagtgaagaaggttttcggatcgtaaagctctgttgttaaggaagaacgagtgtgagaatggaaagttcatg

SEQ ID NO: 20 - DNA, 465 bp, Streptococcus anginosus
cctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatgctgtgacgatacttaaccagaaagggacggctaactacgtgccagcagccgcggtaatacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggggaaatgcgtagatatatggaggaacaccggtggcgaaagcggctctctggtctgtaactgacgctgaggctcgaaagcgtggggagcgaacaggattagatacccgggtagtc

SEQ ID NO: 21 - DNA, 465 bp, Streptococcus anginosus
  misc_feature (2)..(2): n is a, c, g, or t
cntacgggtggcagcagtagggaatcttcggcaatggggggaaccctgaccgagcaacgccgcgtgagtgaagaaggttttcggatcgtaaagctctgttgttaaggaagaacgagtgtgaaaatggaaagttcatactgtgacggtacttaaccagaaagggacggctaactacgtgccagcagccgcggtaatacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagatacccgggtagtc

SEQ ID NO: 22 - DNA, 257 bp, Streptococcus anginosus
cgaacgctggcggcgtgcctaatacatgcaagtaggacgcacagtttataccgtagcttgctacaccataggctgtgagttgcgaacgggtgagtaacgcgtaggtaacctgcctattagagggggataactattggaaacgatagctaataccgcataacagtatgtaacacatgttagatgcttgaaagatgcaattgcatcgctagtagatggacctgcgttgtattagctagtaggtagggtaatggcttacc

SEQ ID NO: 23 - DNA, 463 bp, Streptococcus anginosus
gcacagtttataccgtagcttgctacaccatagactgtgagttgcgaacgggtgagtaacgcgtaggtaacctgcctattagagggggataactattggaaaacgatagctaataccgcataacagtatgtaacacatgttagatgcttgaaagatgcaattgcatcgctagtagatggacctgcgttgtattagctagtaggtagggtaaaggcctacctaggcaacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactac

SEQ ID NO: 24 - DNA, 330 bp, Streptococcus anginosus
agatgcttgaaagatgcaattgcatcgctagtagatgggacctgcgttgtattagctagtaggtagggtaaaggcctaccctaggcaacgatacatagccgacctgagagggtgatcggccacactgggactgagacacggcccaggactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgcc

SEQ ID NO: 25 - DNA, 413 bp, Streptococcus anginosus
gagtaacgcgtaggtaacctgctattagaggggataactattggaaacgatagctaataccgcataacagttatgtaacacatgttagatgcttgaaagatgcaattgcatcgctagtagatggacctgcgttgtattagctagtaggtagggtaatggcctacctaggcaacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgcc

SEQ ID NO: 26 - DNA, 397 bp, Parvimonas sp.
agagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatg

SEQ ID NO: 27 - DNA, 229 bp, Parvimonas sp.
agagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtg

SEQ ID NO: 28 - DNA, 252 bp, Parvimonas sp.
tacggaggatgcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacagg

SEQ ID NO: 29 - DNA, 252 bp, Parvimonas sp.
tacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacagg

SEQ ID NO: 30 - DNA, 252 bp, Parvimonas sp.
tacgtatggggcgagcgttgtccggaattattgggcgtaaagggtacgtaggcggccttttaagtcaggtgtgaaagcgtgaggcttaacctcattaagcacttgaaactggaaggcttgagtgaaggagaggaaagtggaattcctagtgtagcggtgaaatgcgtagatattaggaggaataccggtggcgaaggcgactttctggacttttactgacgctcaggtacgaaagcgtggggagcaaacagg

SEQ ID NO: 31 - DNA, 252 bp, Parvimonas sp.
tacgtatggggcgagcgttgtccggaattattgggcgtaaagggtacgtaggcggtctattaagtcaggtgtgaaagcgtgaggcttaacctcattaagcacttgaaactgatagacttgagtgaaggagaggaaagtggaattcctagtgtagcggtgaaatgcgtagatattaggaggaataccggtggcgaaggcgactttctggacttttactgacgctcaggtacgaaagcgtggggagcaaacagg

SEQ ID NO: 32 - DNA, 251 bp, Parvimonas sp.
tacgtatggggcgagcgttgtccggaattattgggcgtaaagggtacgtaggcggtctattaagtcaggtgtgaaagcgtgaggcttaacctcattaagcacttgaaactgatagacttgagtgaaggagaggaaagtggaattcctagtgtagcggtgaaatgcgtagatattaggaggaataccggtggcgaaggcgactttctggacttttactgacgctcaggtacgaaagcgtggggagcaaacag

SEQ ID NO: 33 - DNA, 252 bp, Parvimonas sp.; identical to SEQ ID NO: 29

SEQ ID NO: 34 - DNA, 252 bp, Parvimonas sp.; identical to SEQ ID NO: 30

SEQ ID NO: 35 - DNA, 120 bp, Parvimonas sp.
tggggaatattgcacaatggggggaaccctgatgcagcgacgccgcgtgagcgaagaaggttttcgaatcgtaaagctctgtcctatgagaagataatgacggtatcataggaggaagcc

SEQ ID NO: 36 - DNA, 438 bp, Parvimonas sp.
cctacgggaggcagcagtggggaatattgcacaatggggggaaccctgatgcagcgacgccgcgtgagcgaagaaggttttcgaatcgtaaagctctgtcctatgagaagataatgacggtatcatagaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtc

SEQ ID NO: 37 - DNA, 439 bp, Parvimonas sp.
cctacgggaggctgcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggccttttaagtcaggtgtgaaagcgtgaggcttaacctcattaagcacttgaaactggaaggcttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtc

SEQ ID NO: 38 - DNA, 363 bp, Parvimonas sp.
  misc_feature (2)..(2): n is a, c, g, or t
cntacgggtggctgcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaa

SEQ ID NO: 39 - DNA, 153 bp, Parvimonas sp.
aactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatac

SEQ ID NO: 40 - DNA, 460 bp, Parvimonas sp.
gcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaaggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatac

SEQ ID NO: 41 - DNA, 257 bp, Parvimonas sp.
ctggcggcgtgcttaacacatgcaagtcgaacgtgattttcatagaagttccttcgggagtggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcga

SEQ ID NO: 42 - DNA, 462 bp, Parvimonas sp.
cgtgcttaacacatgcaagtcgaacgtgatttttgtggaaatctttcgggaatggaaatgaaatgaaagtggcgaacggtgagtaacacgtgagcaacctacctacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgcc

SEQ ID NO: 43 - DNA, 229 bp, Firmicutes sp.
agagtttgatcatggctcaggacgaacgctggcggcgtgcctaacacatgcaagtcgagcggagacagtgagtagcttgctatgagctgttttagcggcggacgggtgagtaacgcgtgagcaacctttcccagacaggggaataacacaccgaaaggtgtactaataccgcataagaccacggaatcacatggttctgaggtaaaagatttatcggtttggggtgggc

SEQ ID NO: 44 - DNA, 252 bp, Firmicutes sp.
tacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaacag

SEQ ID NO: 45 - DNA, 251 bp, Firmicutes sp.
tacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaaca

SEQ ID NO: 46 - DNA, 120 bp, Firmicutes sp.
tggggaatattgggcaatggaggaaactctgacccagcaacgccgcgtggaggaagaaggttttcggatcgtaaactcctgtccttggagacgagtagaagacggtatccaaggaggaag

SEQ ID NO: 47 - DNA, 111 bp, Firmicutes sp.
tggggaatattgggcaatggaggaaactctgacccagcaacgccgcgtggaggaagaaggttttcggatcgtaaactcctgtccttggagacgagtagaagacggtatcca

SEQ ID NO: 48 - DNA, 111 bp, Firmicutes sp.
cggggaatattgggcaatggggggaaccctgacccagcaacgccgcgtggaggaagaaggttttcggatcgtaaactcctgtccttggagacgagtagaagacggtatcca

SEQ ID NO: 49 - DNA, 363 bp, Firmicutes sp.
cctacgggtggctgcagtggggaatattgggcaatggaggaaactctgacccagcaacgccgcgtggaggaagaaggttttcggatcgtaaactcctgtccttggagacgagtagaagacggtatccaaggaggaagccccggctaactacgtgccagcagccgcggtaatacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggc

SEQ ID NO: 50 - DNA, 414 bp, Lachnospira sp.
agagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgcgcgtg

SEQ ID NO: 51 - DNA, 229 bp, Lachnospira sp.
agagtttgatcatggctcaggatgaacgctggcggcgtgcttaacacatgcaagtcgaacgaagcatttaagacggattctttcgggatgaagacttttatgactgagtggcggacgggtgagtaacgcgtgggtaacctgcctcacacagggggatagcagttggaaacggctgataataccgcataagcgcacagtaccgcatggtacagtgtgaaaaactccggtg

SEQ ID NO: 52 - DNA, 229 bp, Lachnospira sp.
agagtttgatcatggctcaggatgaacgctggcggcgtgcttaacacatgcaagtcgaacgaagcatttagaacggattatttcggtatgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtg

SEQ ID NO: 53 - DNA, 229 bp, Lachnospira sp.
agagtttgatcctggctcaggatgaacgctggcggcgtgcttaacacatgcaagtcgaacgaagcatttaagacagattttttcggaatgaagttttttatgactgagtggcggacgggtgagtaacgcgtgggtaacctgcctcacacagggggatagcagttggaaacgactggtaataccgcataagcgcacagcttcgcatgaagcggtgtgaaaaactccggtg

SEQ ID NO: 54 - DNA, 229 bp, Lachnospira sp.
agagtttgatcatggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggagtagcagctggaaacggctggtaataccgcataagcgcacaatgttgcatgacatggtgtgaaaaactaccgg

SEQ ID NO: 55 - DNA, 229 bp, Lachnospira sp.
agagtttgatcatggctcaggatgaacgctggcggcgtgcttaacacatgcaagtcgaacgaagcatttgcgacagatttcttcgggatgaagttgcttatgactgagtggcggacgggtgagtaacgcgtgggtaacctgccttgtactgggggagtagcagctggaaacgggactggtaataccgcataagcgcacaatgttgcatgacatggtgtgaaaaactacc

SEQ ID NO: 56 - DNA, 130 bp, Lachnospira sp.
cgagggaggggtgagtggaattcctagtgtagcggtgaaatgcgtagatattaggaggaacaccagtggcgaaggcggctcactggactgtaactgacactgaggctcgaaagcgtggggagcaaacagg

SEQ ID NO: 57 - DNA, 177 bp, Lachnospira sp.
tatgcgccttgccagcccgctcaggtgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctagagtgcaggagg

SEQ ID NO: 58 - DNA, 177 bp, Lachnospira sp.
tatgcgccttgccagcccgctcaggtgtgccagcagccgcggtaatacgtatggagcaagcgttatccggatttactgggtgtaaagggagtgtaggtggcatcacaagtcagaagtgaaaagcccggggctcaaccccgggactgcttttgaaactgtggagctggagtgcaggag

SEQ ID NO: 59 - DNA, 177 bp, Lachnospira sp.
tatgcgccttgccagcccgctcaggtgtgccagcagccgcggtaatacgtatggagcaagcgttatccggatttactgggtgtaaagggagtgtaggtggccaggcaagtcagaagtgaaagcccggggctcaaccccgggactgcttttgaaactgcagggctagagtgcaggagg

SEQ ID NO: 60 - DNA, 252 bp, Lachnospira sp.
tacgtagggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaggcggcttgctggactgtaactgacactgaggctcgaaagtgtgggtatcaaacag

SEQ ID NO: 61 - DNA, 252 bp, Lachnospira sp.
tacgtatggagcaagcgttatccggatttactgggtgtaaagggagtgtaggtggtatcacaagtcagaagtgaaagcccggggctcaaccccgggactgcttttgaaactgtggaactggagtgcaggagaggtaagtggaattcctagtgtagcggtgaaatgcgtagatattaggaggaacaccagtggcgaaggcggcttactggactgtaactgacactgaggctcgaaagcgtggggagcaaacag

SEQ ID NO: 62 - DNA, 252 bp, Lachnospira sp.
tacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggcttactggacggtaactgacgttgaggctcgaaagcgtggggagcaaacag

SEQ ID NO: 63 - DNA, 251 bp, Lachnospira sp.
tacgtagggggcaagcgttatccggatttactgggtgtaaagggagtgtaggtggtatcacaagtcagaagtgaaagcccggggctcaaccccgggactgcttttgaaactgtggaactggagtgcaggagaggtaagtggaattcctagtgtagcggtgaaatgcgtagatattaggaggaacaccagtggcgaaggcggcctgctggactgttactgacactgaggcacgaaagcgtggggagcaaaca

SEQ ID NO: 64 - DNA, 251 bp, Lachnospira sp.
tacgtatggagcaagcgttatccggatttactgggtgtaaagggagtgtaggtggccaggcaagtcagaagtgaaagcccggggctcaaccccgggactgcttttgaaactgtcagactggagtgcaggagaggtaagcggaattcctagtgtagcggtgaaatgcgtagatattaggaggaacaccagtggcgaaggcggcttgctggactgtaactgacactgaggctcgaaagcgtggggagcaaaca

SEQ ID NO: 65 - DNA, 251 bp, Lachnospira sp.
tacgtatggagcaagcgttatccggatttactgggtgtaaagggagtgtaggtggccatgcaagtcagaagtgaaaatccggggctcaaccccggaactgcttttgaaactgtagagcttgagtgaagtagaggcaggcggaattccccgtgtagcggtgaaatgcgtagagatggggaggaacaccagtggcgaaggcggctcactggactgtaactgacactgaggctcgaaagcgtggggagcaaaca

SEQ ID NO: 66 - DNA, 251 bp, Lachnospira sp.
tacgtagggggcaagcgttatccggatttactgggtgtaaagggagtgtaggtggccatgcaagtcagaagtgaaaatccggggctcaaccctggaactgcttttgaaactgtaaggctggagtgcaggaggggtgagtggaattcctagtgtagcggtgaaatgcgtagagatcggtaggaacaccagtggcgaaggcggcttactggacggcaactgacgttgaggctcgaaagcgtggggagcaaaca

SEQ ID NO: 67 - DNA, 251 bp, Lachnospira sp.
tacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggcttactggacggtaactgacgttgaggctcgaaagcgtggggagcaaaca

SEQ ID NO: 68 - DNA, 251 bp, Lachnospira sp.
tacgggggatgcgagcgttatccggattcattgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaaca

SEQ ID NO: 69 - DNA, 251 bp, Lachnospira sp.
tacgtatggagcaagcgttatccggatttactgggtgtaaagggagtgtaggtggcatcacaagtcagaagtgaaagcccggggctcaaccccgggactgcttttgaaactgtaaggctggagtgcaggaggggtgagtggaattcctagtgtagcggtgaaatgcgtagatattaggaggaacaccagtggcgaaggcggcttgctggactgtaactgacactgaggctcgaaagtgtgggtagcaaaca

SEQ ID NO: 70 - DNA, 251 bp, Lachnospira sp.
tacggaggattcaagcgttatccggatttactgggtgtaaagggagtgtaggtggccatgcaagtcagaagtgaaaatccggggctaccccggaactgcttttgaaactgtgagactagagtgcaggaggggtgagtggaattcctagtgtagcggtgaaatgcgtagatattaggaggaacaccagtggcgaaggcggcttactggacgataactgacgctgaggctcgaaagcgtggggagcaaacagg

SEQ ID NO: 71 - DNA, 120 bp, Lachnospira sp.
tgggaatattgcacaatggaggaaactactgatgcagcgacgccgcgtgagtgaagaagtaattcgttatgtaaagctctatcagcagggaagatagtgacggtacctgactaagaagct

SEQ ID NO: 72 - DNA, 136 bp, Lachnospira sp.
tggggaatattgcacaatggaggaactctgatgcagcgacgccgcgtgagtgaagaagtaattcgttatgtaaagctctatcagcagggaagatagtgacggtacctgactaagaagctccggctaaatacgtgcc

SEQ ID NO: 73 - DNA, 120 bp, Lachnospira sp.
tggggaatattgcacaatggaggaaactctgatgcagcgacgccgcgtgagtgaagaagtagttcgctatgtaaagctctatcagcagggaagatagtgacggtacctgactaagaagct

SEQ ID NO: 74 - DNA, 111 bp, Lachnospira sp.
tgggaatattgcacaatggaggaaactctgatgcagcgacgccgcgtgagtgaagaagtagttcgctatgtaaagctctatcagcagggaagatagtgacggtacctgact

SEQ ID NO: 75 - DNA, 111 bp, Lachnospira sp.
atggaaggaaactctgatgcagcgacgccgcgtgagtgaagaagtaattcgttatagtaaagctctatcagcagggaagatagtgacggtacctgactaagaagctccggc

SEQ ID NO: 76 - DNA, 111 bp, Lachnospira sp.
tggggaaatattgcacaatggaggaaactctgatgcagcgacgccgcgtgagtgaagaagtaattcgttatgtaaagctctatcagcagggaagatagtgacggtacctga

SEQ ID NO: 77 - DNA, 439 bp, Lachnospira sp.
cctacgggggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtc

SEQ ID NO: 78 - DNA, 363 bp, Lachnospira sp.
cctacgggtggctgcagtggggaatattgcacaatggaggaaactctgatgcagcgacgccgcgtgagtgaagaagtagttcgctatgtaaagctctatcagcagggaagatagtgacggtacctgactaagaagctccggctaaatacgtgccagcagccgcggtaatacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcga

SEQ ID NO: 79 - DNA, 298 bp, Lachnospira sp.
cctacgggaggctgcagccgcggtaatacgtatggagcaagcgttatccggatttactgggtgtaaagggagtgtaggtggccatgcaagttagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccccggtagtc

SEQ ID NO: 80 - DNA, 363 bp, Lachnospira sp.
cctacggggggctgcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctagagtgcaggaggggtgagtggaattcctagtgtagcggtgaaatgcgtagatattaggaggaacaccagtggcga

SEQ ID NO: 81 - DNA, 257 bp, Lachnospira sp.
caggatgaacgctggcggcgtgcttaacacatgcaagtcgaacgaagcatttagaacggattacttcggtttgaagttctttatgactgagtggcggacgggtgagtaacgcgtgggtaacctgccttgtactgggggatagcagctggaaacggctggtaataccgcataagcgcacagtgctgcatggcacagtgtgaaaaactccggtggtataagatggacccgcgtctgattagctagttggtgagataaca

SEQ ID NO: 82 - DNA, 257 bp, Lachnospira sp.
ctcaggatgaacgctggcggcgtgcttaacacatgcaagtcgaacgaagcatttaagacagattttttcggaatgaagttttttatgactgagtggcggacgggtgagtaacgcgtgggtaacctgcctcacacagggggatagcagttggaaacgactggtaataccgcataagcgcacagcttcgcatgaagcggtgtgaaaaactccggtggtgtgagatggacccgcgtctgattaggtagttggtgaggtaa

SEQ ID NO: 83 - DNA, 153 bp, Lachnospira sp.
aactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatac

SEQ ID NO: 84 - DNA, 257 bp, Lachnospira sp.
ggctcaggatgaacgctggcggcgtgcttaacacatgcaagtcgaacgaagcatttaagacagattacttcggtttgaagtcttttatgactgagtggcggacgggtgagtaacgcgtgggtaacctgcctcatacagggggatagcagctggaaacggctggtaataccgcataagcgcacagtaccacatggtacagtgtgaaaaactccggtggtatgagatggacccgcgtctgattagctagttggcgggta

SEQ ID NO: 85 - DNA, 257 bp, Lachnospira sp.
ctcaggatgaacgctggcggcgtgcttaacacatgcaagtcgaacgaagcatttaagacggattctttcgggatgaagacttttatgactgagtggcggacgggtgagtaacgcgtgggtaacctgcctcacacagggggatagcagttggaaacggctgataataccgcataagcgcacagtaccgcatggtacagtgtgaaaaactccggtggtgtgagatggacccgcgtctgattagctggttggcggggtaa

SEQ ID NO: 86 - DNA, 478 bp, Lachnospira sp.
aggatgaacgctggcggcgtgcttaacacatgcaagtcgaacgaagcatttagacagattacttcggtttgaagtcttttatgactgagtggcggacgggtgagtaacgcgtggtaacctgcctcatacagggggatagcagctggaaacggctggtaataccgcataagcgcacagtaccacatggtacagtgtgaaaaactccggtggtatgagatggacccgcgtctgattagcttgttggcggggtaacggcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatac

SEQ ID NO: 87 - DNA, 249 bp, Lachnospira sp.
atgaacgctggcggcgtgcttaacacatgcaagtcgaacgaagcatttaagacagattttttcggaatgaagttttttatgactgagtggcggacgggtgagtaacgcgtgggtaacctgcctcacacagggggatagcagttggaaacgactggtaataccgcataagcgcacagcttcgcatgaagcggtgtgaaaaactccggtggtgtgagatggacccgcgtctgattaggtagttggtgaggt

SEQ ID NO: 88 - DNA, 220 bp, Lachnospira sp.
ggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatacgtgcc

SEQ ID NO: 89 - DNA, 204 bp, Lachnospira sp.
agccgactgagaggtgacggccacattgggactgagacacggcccagactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatacgtgcc

SEQ ID NO: 90 - DNA, 399 bp, Lachnospira sp.
gactgagtggcggacgggtgagtaacgcgtgggtaacctgccttgtactggggatagcagcggaaacggctggtaataccgcataagcgcacaatgttgcatgacatggtgtgaaaaactccggtggtatagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgcc

SEQ ID NO: 91 - DNA, 401 bp, Lachnospira sp.
gactgagtggcggacgggtgagtaacgcgtgggtaacctgcctcatacagggggatagcagctggaaacggctggtaataccgcataagcgcacagtaccacatggtacagtgtgaaaaactccggtggtatgagatggacccgcgtctgattagcttgttggcgggtaacggcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatacgtgcc

SEQ ID NO: 92 - DNA, 229 bp, Peptostreptococcus anaerobius
agagtttgatcatggctcaggatgaacgctggcggcgtgcctaacacatgcaagtcgagcgcgtctgatttgatgcttgcattgatgaaagatgagcggcggacgggtgagtaacgcgtgggtaacctgccctatacacatggataacatactgaaaagtttactaatacatgataatatatatttacggcatcgtagatatatcaaagtgttagcggtataggatgga

SEQ ID NO: 93 - DNA, 252 bp, Peptostreptococcus anaerobius
tacgtagggggctagcgttatccggatttactgggcgtaaagggtgcgtaggtggtctttcaagtcggtggttaaaggctacggctcaaccgtagttagcctccgaaactggaagacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacagg

SEQ ID NO: 94 - DNA, 251 bp, Peptostreptococcus anaerobius
tacgtagggggctagcgttatccggatttactgggcgtaaagggtgcgtaggtggtctttcaagtcggtggttaaaggctacggctcaaccgtagttagcctccgaaactggaagacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacag

SEQ ID NO: 95 - DNA, 120 bp, Peptostreptococcus anaerobius
tggggaatattgcacaatgggcgcaagcctgatgcagcaacgccgcgtgaacgatgaaggtcttcggatcgtaaaagttctgttgcaggggaagataatgacggtaccctgtgaggaagc

SEQ ID NO: 96 - DNA, 363 bp, Peptostreptococcus anaerobius
cctacgggtggctgcagtggggaatattgcacaatgggcgcaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtctttcaagtcggtggttaaaggctacggctcaaccgtagttagcctccgaaactggaagacttgagtgcaggagaggaaagtggaattcccagtgtagcggtgaaatgcgtagatattgggaggaacaccagtagcgaa

SEQ ID NO: 97 - DNA, 463 bp, Peptostreptococcus anaerobius
tgaacgctggcggcgtgcctaacacatgcaagtcgagcgcgtctgatttgatgcttgcattaatgaagatgagcggcggacgggtgagtaacgcgtgggtaacctgccctatacacatggataacatactgaaaagtttactaatacatgataatatatatttacggcatcgtagatatatcaaagtgttagcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgaacgatgaaggtcttcggatcgtaaagttctgttgcaggggaagataatgacggtaccctgtgaggaagccccggctaactac

SEQ ID NO: 98 - DNA, 257 bp, Peptostreptococcus anaerobius
cgtcaggatgaacgctggcggcgtgcctaacacatgcaagtcgagcgcgtctgatttgatgcttgcattaatgaaagatgagcggcggacgggtgagtaacgcgtgggtaacctgccctatacacatggataacatactgaaaagtttactaatacatgataatatatatttacggcatcgtagatatatcaaagtgttagcggtataggatggacccgcgtctgattagctagttggtgagataactgcccaccaa

SEQ ID NO: 99 - DNA, 229 bp, Oscillospora sp.
agagtttgatcatggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgc

SEQ ID NO: 100 - DNA, 518 bp, Oscillospora sp.
agagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgaagcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtt

SEQ ID NO: 101 - DNA, 252 bp, Oscillospora sp.
tacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacag

SEQ ID NO: 102 - DNA, 251 bp, Oscillospora sp.
tacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaaca

SEQ ID NO: 103 - DNA, 120 bp, Oscillospora sp.
tggggaatattgggcaatgggcgcaagcctgacccagcaacgccgcgtgaaggaagaaggctttcgggttgtaaacttcttttgtcggggacgaaacaaatgacggtacccgacgaataa

SEQ ID NO: 104 - DNA, 111 bp, Oscillospora sp.
tggggaatattgggcaatgggcgcaagcctgacccagcaacgccgcgtgaaggaagaaggctttcgggttgtaaacttcttttgtcggggacgaaacaaatgacggtaccc

SEQ ID NO: 105 - DNA, 363 bp, Oscillospora sp.
cctacggggggctgcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtgg

SEQ ID NO: 106 - DNA, 257 bp, Oscillospora sp.
tggctcaggatgaacgctggcggcgtgcttaacacatgcaagtcgaacgggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggta

SEQ ID NO: 107 - DNA, 206 bp, Oscillospora sp.
tagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactac

SEQ ID NO: 108 - DNA, 470 bp, Oscillospora sp.
gctggcggcgtgcttaacacatgcaagtcgaacgggtgctcatgacggaggattcgtcacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactac

SEQ ID NO: 109 - DNA, 498 bp, Oscillospora sp.
agtttgatcatggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgaagcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactac

SEQ ID NO: 110 - DNA, 249 bp, Oscillospora sp.
ctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagt

SEQ ID NO: 111 - DNA, 407 bp, Oscillospora sp.
gttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtagcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgcc

SEQ ID NO: 112 - DNA, 494 bp, Oscillospora sp.
ctggctcaggatgaacgctggcggcgtgcttaacacatgcaagtcgaacggggtgctcatgacggaggattcgtccaatggattgagttacctagtggcggacgggtgagtaacgcgtgaggaacctgccttggagagggggataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgcc

SEQ ID NO: 113 - DNA, 229 bp, Dialister sp.
agagtttgatcatggctcaggacgaacgctggcggcgtgcttaacacatgcaagtcgaacgaaaagagggaaagagcttgctctttccggaattgagtggcaaacgggtgagtaacacgtaaacaacctgccttcaggatggggacaacagacggaaacgactgctaataccgaatagcttccagagatcgcatgatccatggaagaaaaggtggcctctacctgtaag

SEQ ID NO: 114 - DNA, 177 bp, Dialister sp.
tatgcgccttgccagcccgctcaggtgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagag

SEQ ID NO: 115 - DNA, 177 bp, Dialister sp.
tatgcgccttgccagcccgctcaggtgtgccagcagccgcggtaatacgtaggtggcaagcgttgtccggaattattgggcgtaaagcgcgcgcaggcggcttcttaagtccatcttaaaagtgcggggcttaaccccgtgatgggatggaaactgagaggctggagtatcggagag

SEQ ID NO: 116 - DNA, 252 bp, Dialister sp.
tacgtaggtggcaagcgttgtccggaattattgggcgtaaagcgcgcgcaggcggcttactaagtccatcttaaaagtgcggggcttaaccccgtgatgggatggaaactggaaagctggagtatcggagaggaaagtggaattcctagtgtagcggtgaaatgcgtagagattaggaagaacaccggtggcgaaggcgactttctggacgaaaactgacgctgaggcgcgaaagcgtgggtatccaacagg

SEQ ID NO: 117 - DNA, 252 bp, Dialister sp.
tacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtggaatgcgtagatatcgggaggaacaccagtggcgaaggcgactttctggacgaaaactgacgctgaggcgcgaaagcgtggggagcaaacagg

SEQ ID NO: 118 - DNA, 252 bp, Dialister sp.
tacgtaggtggcaagcgttgtccggaattattgggcgtaaagcgcgcgcaggcggcttcctaagtccatcttaaaagtgcggggcttaaccccgtgatgggatggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacagg

SEQ ID NO: 119 - DNA, 252 bp, Dialister sp.
tacgtaggtggcaagcgttgtccggaattattgggcgtaaagcgcgcgcaggcggcttcttaagtccatcttaaaagtgcggggcttaaccccgtgatgggatggaaactgggaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacagg

SEQ ID NO: 120 - DNA, 252 bp, Dialister sp.
tacgtaggtggcaagcgttgtccggaattattgggcgtaaagcgcgcgcaggcggccctttaagtccatcttaaaagcgtggggcttaaccccatgatgggatggaaactgaagagctggagtatcggagaggaaagcggaattcctagtgtagcggtgaaatgcgtagagattaggaagaacaccggtggcgaaggcggctttctggacgacaactgacgctgaggcgcgaaagcgtggggagcaaacagg

SEQ ID NO: 121 - DNA, 252 bp, Dialister sp.
tacgtatggtgcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacagg

SEQ ID NO: 122 - DNA, 252 bp, Dialister sp.
tacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagtgcgggtatcgaacagg

SEQ ID NO: 123 - DNA, 251 bp, Dialister sp.
tacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctaatgtagcggtgaaatgcgtagagattaggaagaacaccggtggcgaaggcggcttactggacgataactgacgctgaggctcgaaagcgtggggagcaaacag

SEQ ID NO: 124 - DNA, 251 bp, Dialister sp.
tacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagtgtagcggtgaaatgcgtagatattaggaggaacaccagtggcgaaggcggcttactggacgataactgacgctgaggctcgaaagcgtggggagcaaacag

SEQ ID NO: 125 - DNA, 251 bp, Dialister sp.
tacgtaggtggcaagcgttgtccggaattattgggcgtaaagcgcgcgcaggcggccgtgcaagtccatcttaaaagtgcggggcttaaccccgtgatgggacggaaactgggaagctggagtatcggagaggaaagtggaattcctagtgtagcggtgaaatgcgtagagattaggaagaacaccggtggcgaaggcgactttctggacgacaactgacgctgaggcgcgaaagcgtggggagcaaacag

SEQ ID NO: 126 - DNA, 251 bp, Dialister sp.
tacgtaggtggcaagcgttgtccggaattattgggcgtaaagcgcgcgcaggcggccctttaagtccatcttaaaagcgtggggcttaaccccatgatgggatggaaactgaagagctggagtatcggagaggaaagcggaattcctagtgtagcggtgaaatgcgtagagattaggaagaacaccggtggcgaaggcggctttctggacgacaactgacgctgaggcgcgaaagcgtggggagcaaacag

SEQ ID NO: 127 - DNA, 251 bp, Dialister sp.
tacgtagggggcgagcgttgtccggaattactgggcgtaaagggcgagtaggcggattggcaagttgggagtgaaatgtcggggcttaaccccgtgatgggatggaaactgagaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacag

SEQ ID NO: 128 - DNA, 251 bp, Dialister sp.
tacgtatggtgcaagcgttatccggatttactgggtgtaaagggcgcgcaggcggcgtcgtaagtcggtcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacag

SEQ ID NO: 129 - DNA, 251 bp, Dialister sp.
tacgtaggtggcaagcgttgtccggaattattgggcgtaaagcgcgcgcaggcggcttcccaagtccctcttaaaagtgcggggcttaaccccgtgatgggaaggaaactgtttagctggagtgccggagaggaaagtggaattcctagtgtagcggtgaaatgcgtagagattaggaagaacaccggtggcgaaggcgactttctggacgaaaactgacgctgaggcgcgaaagcgtggggagcaaacag

SEQ ID NO: 130 - DNA, 252 bp, Dialister sp.; identical to SEQ ID NO: 118

SEQ ID NO: 131 - DNA, 251 bp, Dialister sp.
aacgtaggtcacaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacag

SEQ ID NO: 132 - DNA, 120 bp, Dialister sp.
tggggaatcttccgcaatgggcgaaagcctgacggagcaacgccgcgtgagtgatgacggccttcggttgtaaagctctgtgatcggggacgaacggtcagcagacgaataatctgctga

SEQ ID NO: 133 - DNA, 120 bp, Dialister sp.
tggggaatcttccgcaatgggcgaaagcctgacggagcaacgccgcgtgagtgatgacggccttggttgtaagctctgtgaccggggacgaacggtctgtaagctaataccttatagaag

SEQ ID NO: 134 - DNA, 120 bp, Dialister sp.
tggggaatcttccgcaatgggcgaaagcctgacggagcaacgccgcgtgagtgatgacggccttcggttgtaaactctgtgatccgggacgaaaaggcagagtgcgaagaacaaactgca

SEQ ID NO: 135 - DNA, 160 bp, Dialister sp.
tggggaatcttccgcaatgggcgaaagcctgacggagcaacgccgcgtgagtgatgacggccttggttgtaaactctgtgatccgggacgaaaaggcagagtgcgaagaacaaactgcattgacggtaccggaaaagcaagccacggctaactacgtgcc

SEQ ID NO: 136 - DNA, 96 bp, Dialister sp.
tggggaatcttccgcaatgggcgaaagcctgacggagcaacgccgcgtgagtgatgacggccttggttgtaaagctctgtgatcggggacgaacgg

SEQ ID NO: 137 - DNA, 111 bp, Dialister sp.
tggggaatcttccgcaatggggcgaaagcctgacggagcaacgccgcgtgagtgatgacggccttcgggttgtaaaactctgtgatccgggacgaaaaggcagggtgcgaa

SEQ ID NO: 138 - DNA, 111 bp, Dialister sp.
tgggggaatcttccgcaatgggcgaaagcctgacggagcaacgccgcgtgagtgatgacggccttcgggttgtaaaactctgtgatccgggacgaaaaggcagagtgcgaa

SEQ ID NO: 139 - DNA, 99 bp, Dialister sp.
cgggttagtaaagctctgtgagtcggggacgaatggctggtatgctaataccatatcagagtgacggtacccgaatagcaagccacggctaactacgtg

SEQ ID NO: 140 - DNA, 101 bp, Dialister sp.
ttcgggttagtaaaactctgtgatccgggaacgaaaaggcagagtgcgaagaacaaactgcattgacggtaccggaaaagcaagccacggctaactacgtg

SEQ ID NO: 141 - DNA, 465 bp, Dialister sp.
cctacggggggcagcagtggggaatcttccgcaatggacgaaagtctgacggagcaacgccgcgtgagtgaagacggccttcgggttgtaaagctctgtgatccgggacgaaagagcctgaggtaaatagcctaaggaagtgacggtaccggaaaagcaagccacggctaactacgtgccagcagccgcggtaatacgtaggtggcaagcgttgtccggaattattgggcgtaaagcgcgcgcaggcggcttcctaagtccatcttaaaagtgcggggcttaaccccgtgatgggatggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtc

SEQ ID NO: 142 - DNA, 363 bp, Dialister sp.
cctacgggtggctgcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtag

SEQ ID NO: 143 - DNA, 363 bp, Dialister sp.
cctacggggggctgcagtggggaatcttccgcaatgggcgaaagcctgacggagcaacgccgcgtgagtgatgacggccttcgggttgtaaagctctgtgatcggggacgaatgagcagcgtgccaataccacgctgaaatgacggtacccgaaaagcaagccacggctaactacgtgccagcagccgcggtaatacgtaggtggcaagcgttgtccggaattattgggcgtaaagcgcgcgcaggcggtttgttaagtccatcttaaaagtgcggggcttaaccccgtgaggggatggaaactgacagactggagtatcggagaggaaagcggaattcctagtgtagcggtgaaatgcgtag

SEQ ID NO: 144 - DNA, 363 bp, Dialister sp.
cctacgggtggctgcagtggggaatcttccgcaatggacgaaagtctgacggagcaacgccgcgtgagtgatgaaggccttcgggttgtaaagctctttgatccgggacgaacggtctgtgtgctaataccacatagaagtgacggtaccggaagaacaagccacggctaactacgtgccagcagccgcggtaatacgtaggtggcaagcgttgtccggaattattgggcgtaaagcgcgcgcaggcggccctttaagtccatcttaaaagcgtggggcttaaccccatgatgggatggaaactgaagagctggagtatcggagaggaaagcggaattcctagtgtagcggtgaaatgcgtag

SEQ ID NO: 145 - DNA, 429 bp, Dialister sp.
caaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggagggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactac

SEQ ID NO: 146 - DNA, 180 bp, Dialister sp.
cctacgggaggcagcagtggggaatcttccgcaatggacgaaagtctgacggagcaacgccgcgtgagtgaagacggccttcgggttgtaaagctctgtgatccgggacgaaagagcctaaggtaaatagcctaaggaagtgacggtaccggaaaagcaagccacggctaactacgtgcc

SEQ ID NO: 147 - DNA, 249 bp, Dialister sp.
ctggcggcgtgcttaacacatgcaagtcgaacgaaagaggaaagagcttgctcttccggaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggttgcgtctgattagctggttggaggggtaacgg

SEQ ID NO: 148 - DNA, 345 bp, Dialister sp.
agccgcatggcccatggagaaaaggtggcctctacctgtaagctatcgcctgaagagggttgcgtctgattagctggttgaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgcc

SEQ ID NO: 149 - DNA, 474 bp, Dialister sp.
acgggaagagatgaagagcttgctctttatcgaatccagtggcaaacgggtgagtaacacgtaacaacctgccttcaggatggggacaacagacggaacgactgctaataccgaatacgttccacgggccgcatgacctgtggaagaaagggtagcctctacctgtaagctatcgcctgaagaggggtttgcgtctgattaggcagttggtgggtaacggcccaccaaaccaacgatcagtagccggtctgagaggatgaacggccacactggaactgagacacggtccagactcctacgggaggcagcagtggggaatcttccgcaatggacgaaagtctgacggagcaacgccgcgtgagtgaagacggccttcgggttgtaaagctctgtgatccgggacgaaagagcctgaggtgaatagcctaaggaagtgacggtaccggaaaagcaagccacggctaactacgtgcc

SEQ ID NO: 150 - DNA, 538 bp, Eubacterium dolichum
agagtttgatcctggctcaggatgaacgctggcggcatgcctaatacatgcaagtcgaacgaagtcttcaggaagcttgcttccaaaaagacttagtggcgaacgggtgagtaacacgtaggtaacctgcccatgtgtccgggataactgctggaaacggtagctaaaaccggataggtatacggagcgcatgctctgtatattaaagcgcccttcaaggcgtgaacatggatggacctgcgacgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaag

SEQ ID NO: 151 - DNA, 533 bp, Eubacterium dolichum
agagtttgatcctggctcaggatgaacgctggcggcatgcctaatacatgcaagtcgaacgaagtttttaggaaagcttgctttccaaaaagacttagtggcgaacgggtgagtaacacgtagataacctgcccatgtgcccgggataactgctggaaacggtagctaaaaccggataggtggcttcgaggcatctcggagacattaaaatggctacggccatgaacatggatggatctgcggcgcattagctagttggtgaggtaacggcccaccaaggcgacgatgcgtagccgacctgagagggtgaacggccacattgggactgagacacggcccaaactcctacgggaggcagcagtagggaattttcgtcaatggggggaaccctgaacgagcaatgccgcgtgagtgaagaaggtcttcggatcgtaaagctctgttgtaagagaaaacggatcatgtagggaatgacatgatagtgatggtatcttaccagaaagccacggctaactacgtgccagcagccgcgg

SEQ ID NO: 152 - DNA, 472 bp, Eubacterium dolichum
agagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgcaatgggggaaaccctgaacgagcaatgccgcgtgagtgaagaaggtcttcggatcgtaaagctctgttgtaagtgaagaacggctcatagaggaaat

SEQ ID NO: 153 - DNA, 229 bp, Eubacterium dolichum
agagtttgatcatggctcaggatgaacgctggcggcatgcctaatacatgcaagtcgaacgaagtcttcaggaagcttgcttccaaaaagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacat

SEQ ID NO: 154 - DNA, 252 bp, Eubacterium dolichum
tacgtatggtgcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtagatatatggaggaacaccagtggcgaaggcggtcgcctggtctgtaactgacactgatgcacgaaagcgtggggagcaaatagg

SEQ ID NO: 155 - DNA, 252 bp, Eubacterium dolichum
tacgtaggtggcgagcgttatccggaatcattgggcgtaaagggtgcgtaggtggcggattaagtccgtagtaaaaggcattggctcaaccaatgtaagctatggaaactggtcggctggagtgcagaagagggcgatggaattccatgtgtagcggtaaaatgcgtagatatatggaggaacaccagtggcgaaggcggtcgcctggtctgcaactgacactgaggcacgaaagcgtggggagcaaatagg

SEQ ID NO: 156 - DNA, 252 bp, Eubacterium dolichum
tacgtaggtggcaagcgttatccggaatgattgggcgtaaagggtgcgtaggtggtacattaagtctgaagtaaaaggcagcagctcaactgctgttcgctttggaaactggtgaactagagtgcaggagagggcgatggaattccatgtgtagcggtaaaatgcgtagatatatggaggaacaccagtggcgaaggcggtcgcctggcctgtaactgacactgaggcacgaaagcgtggggagcaaatagg

SEQ ID NO: 157 - DNA, 252 bp, Eubacterium dolichum
tacgtaggtggcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtagatatatggaggaacaccagtggcgaaggcggtcgcctggtctgtaactgacactgatgcacgaaagcgtggggagcaaatagg

SEQ ID NO: 158 - DNA, 252 bp, Eubacterium dolichum
tacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaatagg

SEQ ID NO: 159 - DNA, 252 bp, Eubacterium dolichum
tacgtaggtggcgagcgttatccggaatcattgggcgtaaagggtgcgcaggtggtacattaagtccgaagtaaaaggcagcagctcaactgctgttggctttggaaactggtgaactggagtgcaggagagggcgatggaattccatgtgtagcggtaaaatgcgtagatatatggaggaacaccagtggcgaaggcggtcgcctggcctgcaactgacactgaggcacgaaagcgtggggagcaaatagg

SEQ ID NO: 160 - DNA, 251 bp, Eubacterium dolichum
tacgtaggtggcaagcgttatccggaatcattgggcgtaaagggtgcgtaggtggcggattaagtccgtagtaaaaggcattggctcaaccaatgtaagctatggaaactggtcggctggagtgcagaagagggcgatggaattccatgtgtagcggtaaaatgcgtagatatatggaggaacaccagtggcgaaggcggtcgcctggtctgcaactgacactgaggcacgaaagcgtggggagcaaatag

SEQ ID NO: 161 - DNA, 251 bp, Eubacterium dolichum
tacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaatag

SEQ ID NO: 162 - DNA, 252 bp, Eubacterium dolichum; identical to SEQ ID NO: 156

SEQ ID NO: 163 - DNA, 252 bp, Eubacterium dolichum; identical to SEQ ID NO: 157

SEQ ID NO: 164 - DNA, 251 bp, Eubacterium dolichum
tacgtaggtggcgagcgttatccggaatcattgggcgtaaagggtgcgcaggtggtacattaagtccgaagtaaaaggcagcagctcaactgctgttggctttggaaactggtgaactggagtgcaggagagggcgatggaattccatgtgtagcggtaaaatgcgtagatatatggaggaacaccagtggcgaaggcggtcgcctggcctgcaactgacactgaggcacgaaagcgtggggagcaaatag

SEQ ID NO: 165 - DNA, 120 bp, Eubacterium dolichum
tagggaattttcgtcaatggggggaaccctgaacgagcaatgccgcgtgagtgaagaaggtcttcggatcgtaaagctctgttgtaagtgaagaacggtcagtagaggaaatgatactga

SEQ ID NO: 166 - DNA, 120 bp, Eubacterium dolichum
tagggaattttcgtcaatggggggaaccctgaacgagcaatgccgcgtgtgtgaagaaggtcttcggatcgtaaagcactgttgtaagtgaagaatgccatatagaggaaatgctatgtg

SEQ ID NO: 167 - DNA, 120 bp, Eubacterium dolichum
tagggaattttcgtcaatgggggaaacccgtgaacgagcaatgccgcgtgagtgaagaaggtcttcggatcgtaaagctctgttgtaagtgaagaacggctcatagaggaaatgctatgg

SEQ ID NO: 168 - DNA, 120 bp, Eubacterium dolichum
tagggaattttcgtcaatgggggaaaccctgaacgagcaatgccgcgtgagtgaagaaggtcttcggatcgtaaagctctgttgtaagtgaagaacggctcatagaggaaatgctatggg

SEQ ID NO: 169 - DNA, 160 bp, Eubacterium dolichum
tagggaattttcgtcaatggggaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgcc

SEQ ID NO: 170 - DNA, 111 bp, Eubacterium dolichum
tagggaattttcgtcaatgggggaaccctgaacgagcaatgccgcgtgtgtgaagaaggtcttcggatcgtaaagcactgttgtaagtgaagaatgccatatagaggaaat

SEQ ID NO: 171 - DNA, 363 bp, Eubacterium dolichum
cctacggggggctgcagtagggaattttcgtcaatgggggcaaccctgaacgagcaatgccgcgtgagtgaagaacggatcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctg

SEQ ID NO: 172 - DNA, 363 bp, Eubacterium dolichum
cctacggggggctgcagtagggaattttcgtcaatggggggaaccctgaacgagcaatgccgcgtgagtgaagaaggtcttcggatcgtaaagctctgttgtaagtgaagaacggtcagtagaggaaatgatactgaagtgacggtagcttaccagaaagccacggctaactacgtgccagcagccgcggtaatacgtaggtggcgagcgttatccggaatcattgggcgtaaagggtgcgcaggtggtacattaagtccgaagtaaaaggcagcagctcaactgctgttggctttggaaactggtgaactggagtgcaggagagggcgatggaattccatgtgtagcggtaaaatgcgtaga

SEQ ID NO: 173 - DNA, 363 bp, Eubacterium dolichum
cctacggggggctgcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtaga

SEQ ID NO: 174 - DNA, 464 bp, Eubacterium dolichum
cctacgggaggcagcagtagggaattttcgtcaatggggggaaccctgaacgagcaatgccgcgtgagtgaagaaggtcttcggatcgtaaagctctgttgtaagagaaaaacggatcatgtagggaatgacatgatagtgatggtatcttaccagaaagccacggctaactacgtgccagcagccgcggtaatacgtaggtggcaagcgttatccggaatgattgggcgtaaagggtgcgtaggtggtacattaagtctgaagtaaaaggcagcagctcaactgctgttcgctttggaaactggtgaactagagtgcaggagagggcgatggaattccatgtgtagcggtaaaatgcgtagatatatggaggaacaccagtggcgaaggcggtcgcctggcctgtaactgacactgaggcacgaaagcgtggggagcaaataggattagataccccagtagtc

SEQ ID NO: 175 - DNA, 464 bp, Eubacterium dolichum
cctacgggaggcagcagtagggaattttcgtcaatggggggaaccctgaacgagcaatgccgcgtgtgtaaagaaggtcttcggatcgtaaagcactgttgtaagtgaagaatgccacatagaggaaatgctatgtgggtgacggtagcttaccagaaagccacggctaactacgtgccagcagccgcggtaatacgtaggtggcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtagatatatggaggaacaccagtggcgaaggcggtcgcctggtctgtaactgacactgatgcacgaaagcgtggggagcaaataggattagataccctagtagtc

SEQ ID NO: 176 - DNA, 464 bp, Eubacterium dolichum
cctacgggcggctgcagtagggaattttcgtcaatgggggaaaccctgaacgagcaatgccgcgtgagtgaagaaggtcttcggatcgtaaagctctgttgtaagtgaagaacggatcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtc

SEQ ID NO: 177 - DNA, 363 bp, Eubacterium dolichum
cctacggggggctgcagtagggaattttcgtcaatgggggaaaccctgaacgagcaatgccgcgtgagtgaagaaggtcttcggatcgtaaagctctgttgtaagcgaagaacggtccgcataggaaatgatgcgggagtgacggtagcttaccagaaagccacggctaactacgtgccagcagccgcggtaatacgtaggtggcgagcgttatccggaatcattgggcgtaaagggtgcgtaggtggcggattaagtccgtagtaaaaggcattggctcaaccaatgtaagctatggaaactggtcggctggagtgcagaagagggcgatggaattccatgtgtagcggtaaaatgcgtaga

SEQ ID NO: 178 - DNA, 363 bp, Eubacterium dolichum
cctacggggggctgcagtagggaattttcgtcaatggggggaaccctgaacgagcaatgccgcgtgtgtgaagaaggtcttcggatcgtaaagcactgttgtaagtgaagaacgccacatagaggaaatgctatgtgggtgacggtagcttaccagaaagccacggctaactacgtgccagcagccgcggtaatacgtaggtggcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtaga

SEQ ID NO: 179 - DNA, 257 bp, Eubacterium dolichum
tgaacgctggcggcatgcctaatacatgcaagtcgaacggagcgaatatggaagcttgcttccgtaagagctcagtggcgaacgggtgagtaacacgtaggtaacctgcccatgtgcccgggataactgctggaaacggtagctaaaaccggataggtgaataggaggcatctcttattcattaaaggacctgcaaaggtgcgaacatggatggacctgcggcgcattagctggttggagtggtaacggcacaccaa

SEQ ID NO: 180 - DNA, 257 bp, Eubacterium dolichum
gatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccacca

SEQ ID NO: 181 - DNA, 249 bp, Eubacterium dolichum
cgccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactac

SEQ ID NO: 182 - DNA, 496 bp, Eubacterium dolichum
atgaacgctggcgcatgcctaatacatgcaagtcgaacgaagtcttcaggaagcttgcttccaaaaagacttagtggcgaacgggtgagtaacacgtaggtaacctgcccatgtgtccgggataactgctggaaacggtagctaaaaccggataggtatacggagcgcatgctctgtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactac

SEQ ID NO: 183 - DNA, 500 bp, Eubacterium dolichum
aggatgaacgctggcggcatgcctaatacatgcaagtcgaacgaagtttttaggaaagcttgctttccaaaaagacttagtggcgaacgggtgagtaacacgtagataacctgcccatgtgcccgggataactgctggaaacggtagctaaaaccggataggtggcttcgaggcatctcggagacattaaaatggctacggccatgaacatggatggatctgcggcgcattagctagttggtgaggtaacggcccaccaaggcgacgatgcgtagccgacctgagagggtgaacggccacattgggactgagacacggcccaaactcctacgggaggcagcagtagggaattttcgtcaatggggggaaccctgaacgagcaatgccgcgtgagtgaagaaggtcttcggatcgtaaagctctgttgtaagagaaaaacggatcatgtagggaatgacatgatagtgatggtatcttaccagaaagccacggctaactac

SEQ ID NO: 184 - DNA, 422 bp, Eubacterium dolichum
agtggcgaacgggtgagtaacacgtaggtaacctgcccatgtgcccgggataactgctggaaacggtagctaaaaccggataggtatgagggaggcatcttcctcatattaaagcaccttcgggtgtgaacatggatggacctgcggcgcattagctggttggtgaggtaacggcccaccaaggcgatgatgcgtagccgacctgagagggtgaacggccacattgggactgagacacggcccaaactcctacgggaggcagcagtagggaattttcgtcaatggggggaaccctgaacgagcaatgccgcgtgtgtgaagaaggtcttcggatcgtaaagcactgttgtaagtgaagaatgccatatagaggaaatgctatgtgggtgacggtagcttaccagaaagccacggctaactac

SEQ ID NO: 185 - DNA, 497 bp, Eubacterium dolichum
atgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactac

SEQ ID NO: 186 - DNA, 239 bp, Eubacterium dolichum
gatgcgtagccgacctgagagggtgaacggccacattgggactgagacacggcccaaactcctacgggaggcagcagtagggaattttcgtcaatggggggaaccctgaacgagcaatgccgcgtgtgtgaagaaggtcttcggatcgtaaagcactgttgtaagtgaagaatgccatatagaggaaatgctatgtgggtgacggtagcttaccagaaagccacggctaactacgtgcc

SEQ ID NO: 187 - DNA, 495 bp, Eubacterium dolichum
tggcggcatgcctaatacatgacaagtcgaacgaagtcttcaggaagcttgcttccaaaaagacttagtggcgaacgggtgagtaacacgtaggtaacctgcccatgtgtccgggataactgctggaaacggtagctaaaaccggataggtatacggagcgcatgctctgtatattaaagcgcccttcaaggcgtgaacatggatggacctgcgacgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgcc

SEQ ID NO: 188 - DNA, 483 bp, Eubacterium dolichum
gcctaatacatgcaagtcgaacgaagtttttaggaaagcttgcttccaaaagacttagtggcgaacgggtgagtaacacgtagataacctgcccatgtgcccgggataactgctggaaacggtagctaaaaccggataggtggcttcgaggcatctcggagacattaaaatggctacggccatgaacatggatggatctgcggcgcattagctagttggtgaggtaacggcccaccaaggcgacgatgcgtagccgacctgagagggtgaacggccacattgggactgagacacggcccaaactcctacgggaggcagcagtagggaattttcgtcaatggggggaaccctgaacgagcaatgccgcgtgagtgaagaaggtcttcggatcgtaaagctctgttgtaagagaaaaacggatcatgtagggaatgacatgatagtgatggtatcttaccagaaagccacggctaactacgtgcc

SEQ ID NO: 189 - DNA, 400 bp, Eubacterium dolichum
ggtaacctgcccatgtgcccgggataactgctggaaacggtagctaaaaccggataggtatgagggaggcatcttcctcatattaaagcacttcgggtgtgaacatggatggacctgcggcgcattagctggttggtgaggtaacggcccaccaaggcgatgatgcgtagccgacctgagagggtgaacggccacattgggactgagacacggcccaaactcctacgggaggcagcagtagggaattttcgtcaatggggggaaccctgaacgagcaatgccgcgtgtgtgaagaaggtcttcggatcgtaaagcactgttgtaagtgaagaatgccacatagaggaaatgctatgtgggtgacggtagcttaccagaaagccacggctaactacgtgcc

SEQ ID NO: 190 - DNA, 403 bp, Eubacterium dolichum
aggtaacctgcccatgtgtccgggataactgctggaaacggtagctaaaaccggataggtatacagagcgcatgctcagtatattaaagcgcccatcaaggcgtgaacatggatggacctgcggcgcattagctagtggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgcc

SEQ ID NO: 191 - DNA, 229 bp, Fusobacerium sp.
agagtttgatcatggctcaggatgaacgctgacagaatgcttaacacatgcaagtcaacttgaatttgggtttttaacttagatttgggtggcggacgggtgagtaacgcgtaaagaacttgcctcacagctagggacaacatttagaaatgaatgctaatacctgatattatgattttaaggcatcttagaattatgaaagctataagcactgtgagagagctttgcg

SEQ ID NO: 192 - DNA, 401 bp, Fusobacerium sp.
agagtttgatcctggctcaggatgaacgctgacagaatgcttaacacatgcaagtcgactcgagtcttcggacttgggtggcgcacgggtgagtaacgcgtaaagaacttgcctcttagaccgggacaacatctggaaacggatgctaataccggatattatggttttttcgcatggaggaatcatgaaagctagatgcgctaagagagagctttgcgtcccattagctggttggtgaggtaacggcccaccaaggcaatgatgggtagccggcctgagagggtgaacggccacaaggggactgagacacggcccttactcctacgggaggcagcagtggggaatattggacaatggaccgcaagtctgatccagcaattctgtgtgcacgatgacgtttt

SEQ ID NO: 193 - DNA, 229 bp, Fusobacerium sp.
agagtttgatcatggctcaggatgaacgctgacagaatgcttaacacatgcaagtctacttgaatttgggtctttgacttagatttgggtggcggacgggtgagtaacgcgtaaagaacttgcctcacagttagggacaacatttggaaacgaatgctaatacctgatattatgattatacggcatcgtataattatgaaagctatatgcgctgtgagagagctttgcg

SEQ ID NO: 194 - DNA, 229 bp, Fusobacerium sp.
agagtttgatcatggctcaggatgaacgctgacagaatgcttaacacatgcaagtctacttgatccttcgggtgaaggtggcggacgggcgagtaacgcgtaaagaacttgccttacagactgggacaacatttggaaacgaatgctaataccggatattatgactgggttgcatgatctggttatgaaagctatatgcgccgtgagagagctttgcgtcccattagtt

SEQ ID NO: 195 - DNA, 229 bp, Fusobacerium sp.
agagtttgatcatggctcaggatgaacgctgacagaatgcttaacacatgcaagtcaacttgaatttgggtttttaacttaggtttgggtggcggacgggtgagtaacgcgtaaagaacttgcctcacagctagggacaacatttggaaacgagtgctaatacctaatattatgataatagggcatcctataattatgaaagctataagcgctgtgagagagctttgcg

SEQ ID NO: 196 - DNA, 252 bp, Fusobacerium sp.
aacgtaggtcacaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtatcaaacagg

SEQ ID NO: 197 - DNA, 252 bp, Fusobacerium sp.
tacgtatgtcacgagcgttatccggatttattgggcgtaaagcgcgtctaggtggttatataagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctaaagcgcgaaagcgtgggtagcaaacagg

SEQ ID NO: 198 - DNA, 252 bp, Fusobacerium sp.
tacgtatgtcgcaagcgttatccggatttattgggcgtaaagcgcgtctaggcggtttggtaagtctgatgtgaaaatgcggggctcaactccgtattgcgttggaaactgtcaaactagagtactggagaggtaggcggaactacaagtgtagaggtgaaattcgtagatatttgtaggaatgccgatggggaagccagcccactggacagatactgacgctaaagcgcgaaagcgtgggtagcaaacagg

SEQ ID NO: 199 - DNA, 252 bp, Fusobacerium sp.
tacgtatgtcacgagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctaaagcgcgaaagcgtgggtagcaaacagg

SEQ ID NO: 200 - DNA, 252 bp, Fusobacerium sp.
tacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacagg

SEQ ID NO: 201 - DNA, 252 bp, Fusobacerium sp.
tacgtatgtcacgagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgcgaaagcgcgaaagcgtgggtagcaaacagg

SEQ ID NO: 202 - DNA, 252 bp, Fusobacerium sp.
tacgtatgtcgcaagcgttatccggatttattgggcgtaaagcgcgtctaggcggtttggtaagtctgatgtgaaaatgcggggctcaactccgtattgcgttggaaactgccaaactagagtactggagaggtaggcggaactacaagtgtagaggtgaaattcgtagatatttgtaggaatgccgatggggaagccagcctactggacagatactgacgctaaagcgcgaaagcgtgggtagcaaacagg

SEQ ID NO: 203 - DNA, 251 bp, Fusobacerium sp.
tacgtatgtcgcaagcgttatccggatttattgggcgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccgatggggaagccagcccactggacagatactgacgctaaagcgcgaaagcgtgggtagcaaacag

SEQ ID NO: 204 - DNA, 251 bp, Fusobacerium sp.
tacgtagggggcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacag

SEQ ID NO: 205 - DNA, 251 bp, Fusobacerium sp.
tacgtatgtcacgagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctgaagcgcgaaagcgtgggtagcaaacag

SEQ ID NO: 206 - DNA, 252 bp, Fusobacerium sp.; identical to SEQ ID NO: 197

SEQ ID NO: 207 - DNA, 252 bp, Fusobacerium sp.
tacgtatgtcgcgagcgttatccggatttattgggcgtaaagcgcgtctagggggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctgaagcgcgaaagcgtgggtagcaaacagg

SEQ ID NO: 208 - DNA, 252 bp, Fusobacerium sp.
tacgtatgtagcaagcgttatccggatttattgggcgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccgatggggaagccagcccactggacagatactgacgctaaagcgcgaaagcgtgggtagcaaacagg

SEQ ID NO: 209 - DNA, 252 bp, Fusobacerium sp.
tacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctaaagcgcgaaagcgtgggtagcaaacggg

SEQ ID NO: 210 - DNA, 252 bp, Fusobacerium sp.; identical to SEQ ID NO: 200

SEQ ID NO: 211 - DNA, 252 bp, Fusobacerium sp.
tacgtatgtcacgagcgttatccggatttattgggcgtaaagcgcgtctaggtggttatgtaagtctgatgtgaaaatgcagggctcaactctgtattgcgttggaaactgcatgactagagtactggagaggtaagcggaactacaagtgtagaggtgaaattcgtagatatttgtaggaatgccgatggggaagccagcttactggacagatactgacgctaaagcgcgaaagcgtgggtagcaaacagg

SEQ ID NO: 212 - DNA, 252 bp, Fusobacerium sp.; identical to SEQ ID NO: 201

SEQ ID NO: 213 - DNA, 251 bp, Fusobacerium sp.
tacggaggatccgagcgttatccggatttattgggcgtaaagcgcgtctaggcggtttggtaagtctgatgtgaaaatgcggggctcaactccgtattgcgttggaaactgccaaactagagtactggagaggtaggcggaactacaagtgtagaggtgaaattcgtagatatttgtaggaatgccgatggggaagccagcctactggacagatactgacgctaaagcgcgaaagcgtgggtagcaaacag

SEQ ID NO: 214 - DNA, 120 bp, Fusobacerium sp.
tggggaatattggacaatggaccaaaagtctgatccagcaattctgtgtgcacgatgaagtttttcggaatgtaaagtgctttcagttgggaagaagaaagtgacggtaccaacagaaga

SEQ ID NO: 215 - DNA, 120 bp, Fusobacerium sp.
tggggaatattggacaatggaccaaaagtctgatccagcaattctgtgtgcacgatgaagtttttcggtaatgtaaagtagctttctagttgggacgaagtaagtgacggtaccaacaga

SEQ ID NO: 216 - DNA, 120 bp, Fusobacerium sp.
tggggaatattggacaatggaccaaagtctgatccagcaattctgtgtgcacgatgaagttttcggaatgtaaagtgctttcagttgggacgaagtaagtgacggtaccaacagaagaag

SEQ ID NO: 217 - DNA, 120 bp, Fusobacerium sp.
tgaggaatattggtcaatggaccaaaagtctgatccagcaattctgtgtgcacgatgaagtttttcggaatgtaaagtgctttcagttgggacgaagtaagtgacggtaccaacagaaga

SEQ ID NO: 218 - DNA, 120 bp, Fusobacerium sp.
tggggaatattggacaatggaccgagagtctgatccagcaattctgtgtgcacgatgaagtttttcggaatgtaaagtgctttcagttgggaagaaaaaatagacggtaccaacagaaga

SEQ ID NO: 219 - DNA, 120 bp, Fusobacerium sp.
tggggaatattggacaatggaccaaaagtctgatccagcaattctgtgtgcacgatgaagtttttcggaatgtaaaagtgcttttcagttgggacgaagtaagtgacggtaccaacagaa

SEQ ID NO: 220 - DNA, 120 bp, Fusobacerium sp.
tggggaatattggacaatggaccaaaagtctgatccagcaattctgtgtgcacgatgacgtttttcggaatgtaaagtgctttcagttgggaagaaaaatagacggtaccaacagaagaa

SEQ ID NO: 221 - DNA, 120 bp, Fusobacerium sp.
tggggaatattggacaatggaccaaaagtctgatccagcaattctgtgtgcacgtatgaagtttttacggaatgtaaagtgctttcagttgggacgaagaacgtgacggtaccaacagaa

SEQ ID NO: 222 - DNA, 120 bp, Fusobacerium sp.
tggggaatattggacaatggaccaaaagtctgatccagcaattctgtgtgcacgatgaagtttttcggtaatgtaaagtgctttcagttgggacggaagtaagtgacggtaccaacagaa

SEQ ID NO: 223 - DNA, 120 bp, Fusobacerium sp.
tggggaatattggacaatggaccaaaagtctgatccagcaattctgtgtgcacgaagaagtttttcggaatgtaaaagtgctttcagttgggaagaagtcagtgacggtaccaacagaag

SEQ ID NO: 224 - DNA, 139 bp, Fusobacerium sp.
tggggaatattggacaatggaccaagagtctgatccagcaattctgtgtgcacgatgaagtttttcggaatgtaaagtgctttcagttgggaagaaaatagacggtaccaacagaagaagtgacggctaaatacgtgcc

SEQ ID NO: 225 - DNA, 140 bp, Fusobacerium sp.
tggggaatattggacaatggaccaaagtctgatccagcaattctgtgtgcacgatgaagtttttcggaatgtaaagtgctttcagttgggacgaagtaagtgacggtaccaacagaagaagcgacggctaaatacgtgcc

SEQ ID NO: 226 - DNA, 140 bp, Fusobacerium sp.
tggggaatattggacaatggaccgagagtctgatccagcaattctgtgtgcacgatgaagtttttcggaatgtaaagtgctttcagttgggaagaaaaatagacggtaccaacagaagaagtgacggctaaatacgtgcc

SEQ ID NO: 227 - DNA, 111 bp, Fusobacerium sp.
tggggaatattggacaatggaccgagagtctgatccagcaattctgtgtgcacgatgaagtttttcggaatgtaaagtgctttcagttgggaagaaagaaatgacggtacc

SEQ ID NO: 228 - DNA, 111 bp, Fusobacerium sp.
tggggaatattggacaatggaccaaaagtctgatccagcaattctgtgtgcacgatgacgtttttcggaatgtaaagtgctttcagttgggaagaaaaaatgacggtacca

SEQ ID NO: 229 - DNA, 111 bp, Fusobacerium sp.
tgggggaatattggacaatggaccaaaagtctgatccagcaattctgtgtgcacgatgacgtttttcggaatgtaaagtgctttcagtcgggaagaagtaagtgacggtac

SEQ ID NO: 230 - DNA, 111 bp, Fusobacerium sp.
tgggggaatattggacaatggaccaaaagtctgatccagcaattctgtgtgcacgatgaagttttttcggaatgtaaagtgctttcagttgggacgaagtaagtgacggta

SEQ ID NO: 231 - DNA, 111 bp, Fusobacerium sp.
tggggaatattggacaatggaccaaaagtctgatccagcaattctgtgtgcacgatgacgttttcggaatgtaaaagtgctttcagtcgggaagaagcaagtgacggtacc

SEQ ID NO: 232 - DNA, 111 bp, Fusobacerium sp.
tgggggaatattggacaatggaccaaaagtctgatccagcaattctgtgtgcacgatgaagtttttcggaatgtaaaagtgctttcagttgggacgaagtaagtgacggta

SEQ ID NO: 233 - DNA, 111 bp, Fusobacerium sp.
tggggaatattggacaatggaccaaaagtctgatccagcaattctgtgtgcacgatgaagttttcggaatgtaaagtgcttcagttgggacgaagtaagtgacggtaccaa

SEQ ID NO: 234 - DNA, 363 bp, Fusobacerium sp.
cctacgggaggcagcagtggggaatattggacaatggaccgagagtctgatccagcaattctgtgtgcacgatgaagtttttcggaatgtaaagtgctttcagttgggaagaaaaaaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtagatatcacgaagaactccgattg

SEQ ID NO: 235 - DNA, 297 bp, Fusobacerium sp.
cctacgggaggcagcagccgcggtaatacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtc

SEQ ID NO: 236 - DNA, 363 bp, Fusobacerium sp.
  misc_feature (2)..(2): n is a, c, g, or t
cntacgggtggctgcagtggggaatattggacaatggaccaagagtctgatccagcaattctgtgtgcacgatgaagtttttcggaatgtaaagtgctttcagttgggaagaaaaaaatgacggtaccaacagaagaagtgacggctaaatacgtgccagcagccgcggtaatacgtatgtcacaagcgttatccggatttattgggcgtaaagcgtgtctaggtggttatgtaagtctgatgtgaaaatgcagggctcaactctgtattgcgttggaaactgtgtaactagagtactggagaggtaagcggaactacaagtgtagaggtgaaattcgtagatattaggaggaacaccagtgg

SEQ ID NO: 237 - DNA, 443 bp, Fusobacerium sp.
cctacggggggcagcagtggggaatattggacaatggaccgagagtctgatccagcaattctgtgtgcacgatgaagtttttcggaatgtaaagtgctttcagttgggaagaaagaaatgacggtaccaacagaagaagtgacggctaaatacgtgccagcagccgcggtaatacgtatgtcacgagcgttatccggatttattgggcgtaaagtgcgtctaggtggttatgtaagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtc

SEQ ID NO: 238 - DNA, 443 bp, Fusobacerium sp.
  misc_feature (8)..(8): n is a, c, g, or t
cctacggntggcagcagtggggaatattggacaatggaccgagagtctgatccagcaattctgtgtgcacgatgacgtttttcggaatgtaaagtgctttcagttgggaagaaaaaaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctgaagcgcgaaagcgtgggtagcaaacaggattagataccctggtagtc

SEQ ID NO: 239 - DNA, 443 bp, Fusobacerium sp.
cctacgggaggcagcagtggggaatattggacaatggaccaagagtctgatccagcaattctgtgtgcacgatgaagtttttcggaatgtaaagtgctttcagttgggaagaaaaaaatgacggtaccaacagaagaagtgacggctaaatacgtgccagcagccgcggtaatacgtatgtcacaagcgttatccggatttattgggcgtaaagcgcgtctaggtggttatataagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctgaagcgcgaaagcgtgggtagcaaacaggattagataccctggtagtc

SEQ ID NO: 240 - DNA, 442 bp, Fusobacerium sp.
cctacgggtggcagcagtggggaatattggacaatggaccaagagtctgatccagcaattctgtgtgcacgatgaagttttcggaatgtaaagtgctttcagttgggaagaaaaaaatgacggtaccaacagaagaagtgacggctaaatacgtgccagcagccgcggtaatacgtatgtcacgagcgttatccggatttattgggcgtaaagcgcgtctaggtggttatataagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctgaagcgcgaaagcgtgggtagcaaacaggattagatacccctgtagtc

SEQ ID NO: 241 - DNA, 443 bp, Fusobacerium sp.
cctacgggaggcagcagtggggaatattggacaatggaccgagagtctgatccagcaattctgtgtgcacgatgaagtttttcggaatgtaaagtgctttcagttgggaagaaataaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtc

SEQ ID NO: 242 - DNA, 443 bp, Fusobacerium sp.
cctacgggaggcagcagtggggaatattggacaatggaccaaaagtctgatccagcaattctgtgtgcacgatgaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagatacccgggtagtc

SEQ ID NO: 243 - DNA, 443 bp, Fusobacerium sp.
cctacgggaggcagcagtggggaatattggacaatggaccgcaagtctgatccagcaattctgtgtgcacgatgacgtttttcggaatgtaaagtgctttcagtcgggaagaagtcagtgatggtaccgacagaagaagcgacggctaaatacgtgccagcagccgcggtaatacgtatgtcgcaagcgttatccggatttattgggcgtaaagcgcgtctaggcggcaaggaaagtctgatgtgaaaatgcggagctcaactccgtatggcgttggaaactgccttactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtc

SEQ ID NO: 244 - DNA, 442 bp, Fusobacerium sp.
cctacgggggcagcagtggggaatattggacaatggaccgagagtctgatccagcaattctgtgtgcacgatgaagtttttcggaatgtaaagtgctttcagttgggaagaaaaaaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtataactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtc

SEQ ID NO: 245 - DNA, 363 bp, Fusobacerium sp.
cctacgggtggctgcagtggggaatattggacaatggaccaaaagtctgatccagcaattctgtgtgcacgatgaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatgg

SEQ ID NO: 246 - DNA, 363 bp, Fusobacerium sp.
cctacgggtggctgcagtggggaatattggacaatggaccgagagtctgatccagcaattctgtgtgcacgatgaagtttttcggaatgtaaagtgctttcagttgggaagaaataaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatgg

SEQ ID NO: 247 - DNA, 257 bp, Fusobacerium sp.
cagtcgactagagtttgattatggctcaggatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctgattatgaaagctatatgcgctgtgagagagctttgcgtcccattagttagttggtgaggtaacggct

SEQ ID NO: 248 - DNA, 455 bp, Fusobacerium sp.
gatgaacgctgacagaatgcttaacacatgcaagtcaacttgaactcggtttgggtggcggacgggtgagtaacgcgtaaagaacttgcctcacagatagggacaacatttggaaacgaatgctaatacctgatattatgattatatggcatcgtataattatgaaagctatatgcgctgtgagagagctttgcgtcccattagctagttggagaggtaacggctcaccaaggcgatgatgggtagccggcctgagagggtgatcggccacaaggggactgagacacggcccttactcctacgggaggcagcagtggggaatattggacaatggaccaagagtctgatccagcaattctgtgtgcacgatgtaagtttttcggaatgtaaagtgctttcagttgggaagaaaaaatgacggtaccaacagaagaagtgacggctaaatacgtgcc

SEQ ID NO: 249 - DNA, 229 bp, Enterococcus sp.
agagtttgatcatggctcagattgaacgctggcggcatgctttacacatgcaagtcgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatg

SEQ ID NO: 250 - DNA, 252 bp, Enterococcus sp.
tacgtaggtggcaagcgttaatcggaattactgggcgtaaagcgtgcgcaggcggttctgtaagacagatgtgaaatccccgggcttaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacag

SEQ ID NO: 251 - DNA, 253 bp, Enterococcus sp.
tacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacagg

SEQ ID NO: 252 - DNA, 253 bp, Enterococcus sp.; identical to SEQ ID NO: 251

SEQ ID NO: 253 - DNA, 120 bp, Enterococcus sp.
tggggaattttggacaatgggggcaaccctgatccagccatgccgcgtgcaggatgaaggccttcgggttgtaaactgcttttgtcagggacgaaaaggaccgtgttaataccatggtct

SEQ ID NO: 254 - DNA, 111 bp, Enterococcus sp.
tggggaattttggacaatgggggcaaccctgatccagccatgccgcgtgcaggatgaaggccttcgggttgtaaactgcttttgtcagggacgaaaaggaccgtgttaata

SEQ ID NO: 255 - DNA, 111 bp, Enterococcus sp.
tggggaattttggacaatggggggcaacccctgatccagccatgccgcgtgcaggatgaaggccttcgggttgtaaactgcttttgtcagggacgaaaaggaccgtgttaa

SEQ ID NO: 256 - DNA, 363 bp, Enterococcus sp.
cctacgggtggctgcagtggggaattttggacaatgggggcaaccctgatccagccatgccgcgtgcaggatgaaggccttcgggttgtaaactgcttttgtcagggacgaaaaggaccgtgttaataccatggtctgctgacggtacctgaagaataagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtag

SEQ ID NO: 257 - DNA, 298 bp, Enterococcus sp.
cctacggggggctgcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacaggattagataccctagtagtc

SEQ ID NO: 258 - DNA, 257 bp, Enterococcus sp.
gtcgactagagtttgattctggctcagattgaacgctggcggcatgctttacacatgcaagtcgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatgcccgattagctagttggtgag

SEQ ID NO: 259 - DNA, 249 bp, Enterococcus sp
attgaacgctggcggcatgctttacacatgcaagtcgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatgcccgattagctagttggtgaggtaaaggctcaccaaggcg

SEQ ID NO: 260 - DNA, 252 bp, Actinobacteria sp.
cacgtagggggcgagcgttatccggattcattgggcgtaaagcgcgcgcaggcgggcgcctaagcgggacctctaatcttggggctcaacctcaagccgggtcccgaactgggcgcctcgagtgcggtaggggaggtcggaattcccggtgtagcggtggaatgcgcagatatcgggaagaacaccgacggcgaaggcagacctctgggccgccactgacgctgaggcgcgaaagctaggggagcgaacagg

SEQ ID NO: 261 - DNA, 252 bp, Actinobacteria sp.
cacgtagggggcgagcgttatccggattcattgggcgtaaagcgcgcgcaggcggccgcgcaagcggaacctctaacccgagggctcaacccccggccgggttccggactgcgcggctcgggtgcggcagaggcaggcggaactcccggtgtagcggtggaatgcgcagatatcgggaggaacaccgatggcgaaggcagcctgccgggccgccaccgacgctcaggcgcgagagccgggggagcgaacagg

SEQ ID NO: 262 - DNA, 252 bp, Actinobacteria sp.
tacgtagggggcgagcgttatccggattcattgggcgtaaagcgcgcgcaggcggcgcgccaagcggagcttctaatccccgggctcaacccggggccgggctccgaactggcgcgctcgagtgaggtaggggaggtcggaattcccggtgtagcggtggaatgcgcagatatcgggaagaacaccgatggcgaaggcaggcctctgggccttcactgacgctgaggcgcgaaagctgggggagcgaacagg

SEQ ID NO: 263 - DNA, 252 bp, Actinobacteria sp.
tacgtagggggcgagcgttatccggattcattgggcgtaaagcgcgcgtaggcggcgcgccaagcgggacctctaaccccggggctcaaccccgggccgggtcccggactggcgcgctcgagtgcggtagaggcgagtggaattcccggtgtagcggtggaatgcgcagatatcgggaagaacaccgatggcgaaggcagctcgctgggccgccactgacgctgaggcgcgaaagctgggggagcgaacagg

SEQ ID NO: 264 - DNA, 252 bp, Actinobacteria sp.
cacgtggggggcgagcgttgtccggattcattgggcgtaaagcgcgcgcaggcggccgcctaagcgggacctctaaccccggggctcaaccccgggccgggtcccggactgggcggctcgggtgcggtagggggggacggaattcccggtgtagcggtggaatgcgcagatatcgggaggaacaccgacggcgaaggcagtcccctgggccgtcaccgacgctgaggcgcgaaagccgggggagcgaacagg

SEQ ID NO: 265 - DNA, 252 bp, Actinobacteria sp.
tacgtagggggcgagcgttatccggaatcattgggcgtaaagagcgcgtaggcggcccctcaagcgggatctctaatccgagggctcaacccccggccggatcccgaactggggggctcgggtgcggtagaggaggatggaattcccggtgtagcggtggaatgcgcagatatcgggaagaacaccgatggcgaaggcagtcctctgggccgccaccgacgctgaggcgcgaaagccgggggagcgaacagg

SEQ ID NO: 266 - DNA, 252 bp, Actinobacteria sp.
tacgtagggggcgagcgttgtccggattcattgggcgtaaagggcgcgtaggcggcccggaaggccgggggtgaaagcgcggggcccaaccccgcaagcgcccccgggacccccgggctcgggtcccgcagggggtggcggaactcccggtgtagcggtggaatgcgcagatatcgggaggaacaccggtggcgaaggcggccacctgggcggcgaccgacgctgaggcgcgagagccgggggagcgaacag

SEQ ID NO: 267 - DNA, 252 bp, Actinobacteria sp.
cacgtagggggcgagcgttatccggattcattgggcgtaaagcgcgcgtaggcggccgctcgagcgggacctctaacccgggggctcaacctccggccgggtcccggaccgtgcggctcgggtgcggtaggggcaggcggaactccaagtgtagcggtgaaatgcgcagatatttggaggaacaccgatggcgaaggcagcctgctgggccgccaccgacgctgaggcgcgaaagccgggggagcgaacagg

SEQ ID NO: 268 - DNA, 252 bp, Actinobacteria sp.
tacgtagggagcgagcgttatccggattcattgggcgtaaagcgcgcgtaggcgggcgtttaagcggaatctctaatccgagggctcaacccccggccggattccgaactggacgcctcgagttcggtagaggaagatggaattcccggtgtagcggtggaatgcgcagatatcgggaagaacaccgatggcgaaggcagtcttctgggccgcgactgacgctgaggcgcgaaagctgggggagcgaacagg

SEQ ID NO: 269 - DNA, 252 bp, Actinobacteria sp.
gacgtagggggcgagcgttgtccggattcattgggcgtaaagcgcgcgcaggcggcccggcaggccgggggtgaaaacgcggggctcaaccccgcgcctgcccccggaaccgccgggctcgggtcccgcaggggacggcggaactcccggtgtagcggtggaatgcgcagatatcgggaggaacaccggcggcgaaggcggccgtctgggcggagaccgacgctgaggcgcgagagccgggggagcgaacag

SEQ ID NO: 270 - DNA, 251 bp, Actinobacteria sp.
tacgtatggggcgagcgttatccggattcattgggcgtaaagcgcgcgtaggcggctgtgcaagcgggtgtcttaaatccgggggctcaacctccggctggaccccgaactgcacggctcgagttcggtaggggtggtcggaattcccagtgtagcggtgaaatgcgcagatattgggaagaacaccgatggcgaaggcagaccactgggccgcaactgacgctgaggtgcgaaagccgggggagcgaaca

SEQ ID NO: 271 - DNA, 251 bp, Actinobacteria sp.
tacgtagggggcgagcgttatccggattcattgggcgtaaagcgctcgtaggcggcctgctaggtcgggagtcaaatgcgggggcccaacccccggccgctcccgataccggcgggcttgagtttggtaggggaaggcggaattcccggtgtagcggtggaatgcgcagatatcgggaagaacaccggtggcgaaggcggccttctgggccacaactgacgctgaggagcgaaagctgggggagcgaacag

SEQ ID NO: 272 - DNA, 251 bp, Actinobacteria sp.
tacgtagggggcgagcgttatccggattcattgggcgtaaagcgcgcgcaggcggcctgccaagcgggatctccaatccgagggcccaacccccggccggatcccgaactgggaggctcgagtacggtagaggaggatggaattcccagtgtagcggtggaatgcgcagatattgggaagaacaccgatggcgaaggcagtcctctgggccggaactgacgctgaggcgcgaaagctgggggagcgaacag

SEQ ID NO: 273 - DNA, 251 bp, Actinobacteria sp.
tacgtagggggcgagcgttatccggattcattgggcgtaaagcgcgcgtaggcgggcctctaagcggaacctctaacccgagggctcaacccccggccgggttccgaactggaggcctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacag

SEQ ID NO: 274 - DNA, 251 bp, Actinobacteria sp.
tacgtagggggcgagcgttatccggaatcattgggcgtaaagagcgcgtaggcggcccctcaagcgggatctctaatccgagggctcaacccccggccggatcccgaactggggggctcgggtgcggtagaggaggatggaattcccggtgtagcggtggaatgcgcagatatcgggaagaacaccgatggcgaaggcagtcctctgggccgccaccgacgctgaggcgcgaaagccgggggagcgaacag

SEQ ID NO: 275 - DNA, 251 bp, Actinobacteria sp.
tacgtatggtgcgagcgttatccggattcattgggcgtaaagcgcgcgtaggcggcctgttaagcaaggtcttaaatcttggggctcaacctcaagccggactttgaactggcaggctcgagtgtggtagaggaaagtggaattcccagtgtagcggtgaaatgcgcagatattgggaagaacaccgatggcgaaggcagctttctgggccatcactgacgctgaggtgcgaaagctaggggagcaaacag

SEQ ID NO: 276 - DNA, 120 bp, Actinobacteria sp.
tggggaatcttgcgcaatgggcggaagcctgacgcagcgacgccgcgtgcgggaggaaggccctcgggtcgtaaaccgctttcagcagggacgaggccgcaaggtgacggtacctgcaga

SEQ ID NO: 277 - DNA, 120 bp, Actinobacteria sp.
tggggaattttgcgcaatgggggcaaccctgacgcagcaacgccgcgtgcgggacgacggccttcgggttgtaaaccgctttcagcagggaagaaattcgacggtacctgcagaagaagc

SEQ ID NO: 278 - DNA, 120 bp, Actinobacteria sp.
tggggaattttgcgcaatggggggaaccctgacgcagcaacgccgcgtgcgggacgaaggccttcgggttgtaaaccgctttcagcagggaagattcagacggtacctgcagaagaagct

SEQ ID NO: 279 - DNA, 120 bp, Actinobacteria sp.
tggggaattttgcgcaatgggggcaaccctgacgcagcaacgccgcgtgcgggacggaggccttcgggtcgtaaaccgctttcagcagggaagaactttgactgtacctgcagaagaagc

SEQ ID NO: 280 - DNA, 120 bp, Actinobacteria sp.
tggggaattttgcgcaatgggggaaaccctgacgcagcaacgccgcgtgcgggacgaaggccttcgggtcgtaaaccgctttcagcagggaagaacactgacggtacctgcagaagaagc

SEQ ID NO: 281 - DNA, 120 bp, Actinobacteria sp.
tggggaattttgcgcaatgggggaaaccctgacgcagcaacgccgcgtgcgggacgacggccttcgggttgtaaaccgctttcagcagggaagaacaacgacggtacctgcagaagaagc

SEQ ID NO: 282 - DNA, 137 bp, Actinobacteria sp.
tggggaattttgcgcaatgggggaaaccctgacgcagcaacgccgcgtgcgggatgacggccttcggttgtaaaccgctttcagcagggaagaaattcgacggtacctgcagaagaagctccggctaactacgtgcc

SEQ ID NO: 283 - DNA, 136 bp, Actinobacteria sp.
tggggaattttgcgcaatgggggaaaccctgacgcagcaacgccgcgtgcgggacgacggccttcggttgtaaaccgcttcagcagggaagaaattcgacggtacctgcagaagaagctccggctaactacgtgcc

SEQ ID NO: 284 - DNA, 136 bp, Actinobacteria sp.
tggggaattttgcgcaatggggggaaccctgacgcagcaacgccgcgtgcgggacgaaggccttcgggttgtaaccgctttcagcagggaagacatagacggtacctgcagaagaagctccggctaactacgtgcc

SEQ ID NO: 285 - DNA, 137 bp, Actinobacteria sp.
tggggaatcttgcgcaatgggggcaaccctgacgcagcgacgccgcgtgcgggacgaaggccttcgggtcgtaaaccgctttcagcagggacgagacaagacggtacctgcagaagaagcccggctaactacgtgcc

SEQ ID NO: 286 - DNA, 137 bp, Actinobacteria sp.
tggggaattttgcgcaatggggggaaccctgacgcagcaacgccgcgtgcgggatgaaggccttcgggttgtaaaccgctttcagcagggaagatagtgacggtacctgcagaagaagccccggctaactacgtgcc

SEQ ID NO: 287 - DNA, 111 bp, Actinobacteria sp.
tggggaattttgcgcaatgggggcaaccctgacgcagcaacgccgcgtgcgggacgacggccttcgggttgtaaaccgctttcagcagggaagaaattcgacggtacctgc

SEQ ID NO: 288 - DNA, 111 bp, Actinobacteria sp.
tggggaattttgcgcaatggggggaaccctgacgcagcaacgccgcgtgcgggacgaaggccttcgggttgtaaaccgctttcagcagggaagattcagacggtacctgca

SEQ ID NO: 289 - DNA, 363 bp, Actinobacteria sp.
cctacgggtggcagcagtggggaatcttgcgcaatgggggaaaccctgacgcagcaacgccgcgtgcgggatgacggccttcgggttgtaaaccgctttcagcagggaagaacctatgacggtacctgcagaagaagccccggctaactacgtgccagcagccgcggtaatacgtagggggcgagcgttatccggattcattgggcgtaaagcgcgcgcaggcggcctgccaagcgggatctccaatccgagggcccaacccccggccggatcccgaactgggaggctcgagtacggtagaggaggatggaattcccagtgtagcggtggaatgcgcagatattgggaagaacaccggtggcg

SEQ ID NO: 290 - DNA, 257 bp, Actinobacteria sp.
cctatcccctgtgtgccttggcagtcgactagagtttgattctggctcaggatgaacgctggcggcgtgcttaacacatgcaagtcgaacgaataacccacctccgggtggttatagagtggcgaacgggtgagtaacacgtgaccaacctacctctcactccgggataacccagagaaatctgcgctaataccggatactccgggcacctcgcatggggagcccgggaaagccccgacggtgggagatggggtcgc

SEQ ID NO: 291 - DNA, 407 bp, Proteobacteria sp.
agagtttgatcatggctcagattgaacgctggcggcaggcctaacacatgcaagtcgaacggtaacaggaatcagcttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaaga

SEQ ID NO: 292 - DNA, 461 bp, Proteobacteria sp.
agagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcctcgggttgtaagtactttcagcgaggaggaaggcattaaggttaataacct

SEQ ID NO: 293 - DNA, 390 bp, Proteobacteria sp.
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataatgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagcca

SEQ ID NO: 294 - DNA, 406 bp, Proteobacteria sp.
agagtttgatcctggctcagattgaacgctggcggcaggcctaacacatgcaagtcgaacggtaacaggaagcagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaag

SEQ ID NO: 295 - DNA, 411 bp, Proteobacteria sp.
agagtttgatcctggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcct

SEQ ID NO: 296 - DNA, 481 bp, Proteobacteria sp.
agagtttgatcctggctcagattgaacgccggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaaggtactttcagcgggaggaagggagtaaagttaataacctttgctcattgacgtta

SEQ ID NO: 297 - DNA, 413 bp, Proteobacteria sp.
agagtttgatcatggctcagattgaacgctggcggcaggcctaacacatgcaagtcgaacggtaacaggaatcagcttgctgattcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcct

SEQ ID NO: 298 - DNA, 406 bp, Proteobacteria sp.
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaag

SEQ ID NO: 299 - DNA, 412 bp, Proteobacteria sp.
agagtttgatcctggctcagattgagcgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttagggcctcttgccatcggatgtgcccaaatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcc

SEQ ID NO: 300 - DNA, 412 bp, Proteobacteria sp.
agagtttgatcctggctcagattgaacgctggcggcaggcctaacacatgcaagtcgaacggtaacaggaatcagcttgctgattcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcc

SEQ ID NO: 301 - DNA, 451 bp, Proteobacteria sp.
agagtttgatcctggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcctcggttgtaagtacttctagcgggaggaaggtgttgaggtt

SEQ ID NO: 302 - DNA, 321 bp, Proteobacteria sp.
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagac

SEQ ID NO: 303 - DNA, 411 bp, Proteobacteria sp.
agagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaacggtagctaataccgcataacgtcgcaagaccaaagagggggaccttcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcc

SEQ ID NO: 304 - DNA, 410 bp, Proteobacteria sp.
agagtttgatcatggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcc

SEQ ID NO: 305 - DNA, 407 bp, Proteobacteria sp.
cagagtttgatcaggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaaga

SEQ ID NO: 306 - DNA, 358 bp, Proteobacteria sp.
agagtttgatcctggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaat

SEQ ID NO: 307 - DNA, 405 bp, Proteobacteria sp.
agagtttgatcatggctcagattgaacgctggcggcaggcctaacacatgcaagtcgaacggtaacaggaagcagcttgctgcttcgctgacgagtggcggacgggtgagtaatgtctgggaagctgcctgatggagggggataactactggaaacggtagctaataccgcataatgtcgcaagaccaaagagggggaccttcgggcctcttgcatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaag

SEQ ID NO: 308 - DNA, 413 bp, Proteobacteria sp.
agagtttgatcctggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagttagtaggcggggtaatggcccacctaggcgacgatccctagctggtctgagaggatgaccagccacactggaactgagacacggtccagactcctacgggaggcagcagtggggaatatgcacaatgggcgcaagcctgatgcagccatgccgcgtgtatgaagaaggcctcgg

SEQ ID NO: 309 - DNA, 403 bp, Proteobacteria sp.
agagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtgggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatga

SEQ ID NO: 310 - DNA, 479 bp, Proteobacteria sp.
agagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcggttgtaagtacttcagcgggaggaagggagtaaagttaatatctttgctcattgacgttaccc

SEQ ID NO: 311 - DNA, 412 bp, Proteobacteria sp.
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagcttgttggtgggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcct

SEQ ID NO: 312 - DNA, 229 bp, Proteobacteria sp.
agagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgccca

SEQ ID NO: 313 - DNA, 229 bp, Proteobacteria sp.
agagtttgatcctggctcagattgaacgctggcggcaggcctaacacatgcaagtcgaacggtaacaggaagcagcttgctgctttgctgacgagtggcggacgggtgagtaatgtctgggaaactgcctgatggagggggataacgtactggaaacggtggctaataccgcataacgtcgcaagaccaaagagggggaccttcggggcctcttgccatcagatgtgcc

SEQ ID NO: 314 - DNA, 229 bp, Proteobacteria sp.
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggacccgtcgggccgtcttgccatcggatgtgcc

SEQ ID NO: 315 - DNA, 229 bp, Proteobacteria sp.
agagtttgatcctggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataatgtcgcaagatcaaagaggggggaccttcgggcctcttgccatcggatgtgccc

SEQ ID NO: 316 - DNA, 229 bp, Proteobacteria sp.
agagtttgatcatggctcaggttgaacgctggcggcaggcctaacacatgcaagtcgaacggtaacagggatcagcttgctgattcgctgacgagtggcggacgggtgagtaatgtctgggaaactgcctgatggagggggataactactggaaacggtagctaataccgcataacgtcgcaagaccaaagagggggaccttcgggcctcttgccatcggatgtgccca

SEQ ID NO: 317 - DNA, 229 bp, Proteobacteria sp.
agagtttgatcctggctcagattgaacgctggcggcaggcctaacacatgcaagtcgaacggtagcacaaaggagcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcggatgtgccca

SEQ ID NO: 318 - DNA, 229 bp, Proteobacteria sp.
agagtttgatcctggctcagattgaacgctggcggcaggcctaacacatgcaagtcgaacggtaacaggaaacagcttgctgtttcgctgacgagtggcggacgggtgagtaatgtctgggaaactgcctgatggaggggataacgtactggaaacggtagctaataccgcataacgtcgcaagaccaaagagggggaccttcgggcctcttgccatcggatgtgccca

SEQ ID NO: 319 - DNA, 177 bp, Proteobacteria sp.
tatgcgccttgccagcccgctcaggtgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtaga

SEQ ID NO: 320 - DNA, 130 bp, Proteobacteria sp.
gtctcgtagaggggggtagaattccaggcgtagcggtgaaatgcgtagagatctggaggaataccggtggcgaaggcggcccctggacgaagactgacgctcaggtgcgaaagcgtggggagcaaacagg

SEQ ID NO: 321 - DNA, 118 bp, Proteobacteria sp.
gggtagaattccaggtgtagcggtggaatgcgtagagatctggaggaataccggtggcgaaggcggccccctggacgaagactgacgctcaggtgcgaaagcgtggggagcaaacagg

SEQ ID NO: 322 - DNA, 177 bp, Proteobacteria sp.
tatgcgccttgccagcccgctcaggtgtgccagcagccgcggtaatacggagggtgcaagcgttaatcggaattactgggcgtaaagcgcacgcaggcggtttgttaagtcagatgtgaaaatccccgggctcaacctgggaactgcatctgatactggcaagcttgagtctcgtag

SEQ ID NO: 323 - DNA, 114 bp, Proteobacteria sp.
tagaattccaggtgtagcggtgaaatgcgtagagatctggaggaataccggtggcgaaggcggcccctggacgaagactgacgctcaggtgcgaaagcgtggggagcaaacagg

SEQ ID NO: 324 - DNA, 252 bp, Proteobacteria sp.
tacgtagggggcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacag

SEQ ID NO: 325 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaagcttgagtctcgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 326 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcctccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 327 - DNA, 253 bp, Proteobacteria sp.
tacggagggtgcaagcgttaatcggaattactgggcgtaaagcgcacgcaggcggtctgtcaagtcggatgtgaaatccccgggcttaacctgggaactgcattcgaaactggcaggctggagtcttgtagaggtgggtagaattccaggtgtagcggtgaaatgcgtagagatctggaggaataccggtggcgaaggcggccccctggacaaagactgacgctcaggtgcgaaagcgtggggagcaaacagg

SEQ ID NO: 328 - DNA, 253 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaaaactgacgctcaggtgcgaaagcgtggggagcaaacagg

SEQ ID NO: 329 - DNA, 253 bp, Proteobacteria sp.
tacggagggtgcaagcgttaatcggaattactgggcgtaaagcgcacgcaggcggtctgtcaagtcttatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 330 - DNA, 253 bp, Proteobacteria sp.
tacggagggtgcaagcgttaatcggaattactgggcgtaaagcgcacgcaggcggtctgtcaagtcagatgtgaaatccccgggctcaacctgggaactgcatccgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 331 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtggcggtgaaatgcgtagagatctggaggaataccggtggcgaaggcggccccctggacaaagactgacgctcaggtgcgaaagcgtggggagcaaacagg

SEQ ID NO: 332 - DNA, 253 bp, Proteobacteria sp.
tacggagggtgcaagcgttaatcggaattactgggcgtaaagcgcacgcaggcggtttgtcaagtcggatgtgaaatccccgggctcaacctgggaactgcattcgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 333 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatccgtagagatctggaggaataccggtggcgaaggcggccccctggacaaagactgacgctcaggtgcgaaagcgtggggagcaaacagg

SEQ ID NO: 334 - DNA, 253 bp, Proteobacteria sp.
tacggagggtgcaagcgttaatcggaattactgggcgtaaagcgcacgcaggcggtttgttaagtcagatgtgaaatccccgagctcaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 335 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcagactagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 336 - DNA, 253 bp, Proteobacteria sp.
tacggagggtgcaagcgttaatcggaattactgggcgtaaagcgcacgcaggcggtctgtcaagtcggatgtgaaatccccgggctcaacccgggaactgcatccgaaactggcaggcttgagtcttgtagaggggggtagaattccaggtgtagcggtgaaatgcgtagagatctggaggaataccggtggcgaaggcggccccctggacaacgactgacgctcaggtgcgaaagcgtggggagcaaacagg

SEQ ID NO: 337 - DNA, 253 bp, Proteobacteria sp.
tacggagggtgcaagcgttaatcggaattactgggcgtaaagcgcacgcaggcggtctgttaagtcagatgtgaaatccccgggctcaacctgggaactgcatctgaaactggcaggctagagtctcgtagaggggggtagaattccaggtgtagcggtgaaatgcgtagagatctggaggaataccggtggcgaaggcggccccctggaccaagactgacgctcaggtgcgaaagcgtggggagcaaacagg

SEQ ID NO: 338 - DNA, 253 bp, Proteobacteria sp.
tacggagggtgcaagcgttaatcggaattactgggcgtaaagcgcacgcaggcggtttgttaagtcagatgtgaaatccccgggctcaacctgggagctgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 339 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactgacaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 340 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcgatgaaatgcgtagagatctggaggaataccggtggcgaaggcggccccctggacaaagactgacgctcaggtgcgaaagcgtggggagcaaacagg

SEQ ID NO: 341 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcatctgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 342 - DNA, 253 bp, Proteobacteria sp.
tacggagggtgcaagcgttaatcggaattactgggcgtaaagcgcacgcaggcggtttgttaagtcagatgtgaaatccccgggctcaacctgggaactgcatttgatactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 343 - DNA, 253 bp, Proteobacteria sp.
tacggagggtgcaagcgttaatcggaattactgggcgtaaagcgcacgcaggcggtttgttaagtcagatgtgaaatcccggggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 344 - DNA, 253 bp, Proteobacteria sp.
tacggagggtgcaagcgttaatcggaattactgggcgtaaagcgcacgcacgcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 345 - DNA, 252 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacag

SEQ ID NO: 346 - DNA, 251 bp, Proteobacteria sp.
tacgtagggggcaagcgttatccggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 347 - DNA, 251 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagatattaggaggaacaccagtggcgaaggcggcttactggacgaagactgacgctcaggtgcgaaagcgtggggagcaaaca

SEQ ID NO: 348 - DNA, 251 bp, Proteobacteria sp.
aacgtagggtgcaagcgttgtccggaattactgggcgtaaagcgcacgcaggcggtctgtcaagtcggatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 349 - DNA, 251 bp, Proteobacteria sp.
aacgtaggtcacaagcgttgtccggaattactgggcgtaaagcgcacgcaggcggtctgtcaagtcggatgtgaaatccccgggctcaacctgggaactgcattcgaaactggcaggctggagtctcgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 350 - DNA, 251 bp, Proteobacteria sp.
tacggagggtgcaagcgttaatcggaattactgggcgtaaagcgcacgcaggcggaccggaaagttgggggtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 351 - DNA, 251 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggtaggcggaattcccggtgtagcggtgaaatgcgtagagatcgggaggaacaccagtggcgaaggcggcctactggacgaagactgacgctcaggtgcgaaagcgtggggagcaaaca

SEQ ID NO: 352 - DNA, 251 bp, Proteobacteria sp.
tacggagggtgcaagcgttaatcggaatcactgggcgtaaagcgcacgtaggctgtatatcaagtcaagggtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 353 - DNA, 251 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaagaacaccaaaggcgaaggcagtctcctggacgaagactgacgctcaggtgcgaaagcgtggggagcaaaca

SEQ ID NO: 354 - DNA, 251 bp, Proteobacteria sp.
tacggagggggctagcgttgttcggaattactgggcgtaaagcgcacgcaggcggtctgtcaagtcggatgtgaaatccccgggctcaacctgggaactgcatccgaaactggcaggctagagtctcgtagaggggggtagaattccaggtgtagcggtgaaatgcgtagagatctggaggaataccggtggcgaaggcggccccctggacgaagactgacgctcaggtgcgaaagcatggggagcaaaca

SEQ ID NO: 355 - DNA, 251 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattctcggtgtagcagtgaaatgcgtagatatatggaagaacatcagtggcgaaggcggctgtctggaccggtattgacgctgaggcgcgaaagcgtggggagcaaaca

SEQ ID NO: 356 - DNA, 251 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagatatcgggaggaacaccagtggcgaaggcggcctactgggcactaactgacgctcaggtgcgaaagcgtggggagcaaaca

SEQ ID NO: 357 - DNA, 251 bp, Proteobacteria sp.
tccggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagatatcgagaggaacactcgtggcgaaggcgggttcctggacgaagactgacgctcaggtgcgaaagcgtggggagcaaaca

SEQ ID NO: 358 - DNA, 251 bp, Proteobacteria sp.
tacggaggatccgagcgttatccggatttattgggtttaaagggagcgtaggcggctttgcaagtcagatgtgaaatctatgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtgggtatccaaca

SEQ ID NO: 359 - DNA, 251 bp, Proteobacteria sp.
tacgtaggtcccgagcgttgtccggatttattgggcgtaaagggagcgtaggcggatgattaagtgggatgtgaaatacccgggctcaacttgggaactgcatctgatactggcaagcttgagtctcgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 360 - DNA, 251 bp, Proteobacteria sp.
tacggagggtgcaagcgttaatcggaattactgggtgtaaagggagcgcaggcggaaggctaagtctgatgtgaaagcccggggctcaacctgggaactgcattcgaaactggcaggctggagtcttgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 361 - DNA, 253 bp, Proteobacteria sp.; identical to SEQ ID NO: 325

SEQ ID NO: 362 - DNA, 253 bp, Proteobacteria sp.
tacggagggtgcaagcgttactcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 363 - DNA, 253 bp, Proteobacteria sp.; identical to SEQ ID NO: 326

SEQ ID NO: 364 - DNA, 253 bp, Proteobacteria sp.
tacggagggtgcaagcgttaatcggaattactgggcgtaaagcgcacgcaggcggtttattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 365 - DNA, 253 bp, Proteobacteria sp.
tacggagggtgcaagcgttaatcggaattactgggcgtaaagcgcacgcaggcggtctgtcaagtcggatgtgaaatccccgggcttaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 366 - DNA, 253 bp, Proteobacteria sp.; identical to SEQ ID NO: 328

SEQ ID NO: 367 - DNA, 253 bp, Proteobacteria sp.
  misc_feature (3)..(3): n is a, c, g, or t
tanggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcacgtgcgaaagcgtggggagcaaacagg

SEQ ID NO: 368 - DNA, 253 bp, Proteobacteria sp.
tacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcaggcccctggacgaagactgacgctcaggtgcgaaagcgtggggagcaaacagg

SEQ ID NO: 369 - DNA, 253 bp, Proteobacteria sp.
tacggagggtgcaagcgttaatcggaattactgggcgtaaagcgcacgcaggcggtttgttaagtcagatgtgaaatccccgagcttaacttgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 370 - DNA, 253 bp, Proteobacteria sp.
tacggagggtgcaagcgttaatcggaattactgggcgtaaagcgcacgcaggcggtttgttaagtcagatgtgaaatccccgggctcaacctgggaactgcattcgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 371 - DNA, 253 bp, Proteobacteria sp.
tacggagggtgcaagcgttaatcggaattactgggcgtaaagcgcacgcaggcggtctgtcaagtcagatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 372 - DNA, 253 bp, Proteobacteria sp.
tacggagggtgcaagcgttactcggaattactgggcgtaaagcgcacgcaggcggtctgtcaagtcggatgtgaaatccccgggctcaacctgggaactgccttcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 373 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgcaactggcaggctggagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 374 - DNA, 253 bp, Proteobacteria sp.; identical to SEQ ID NO: 331

SEQ ID NO: 375 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgatactggcaagcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 376 - DNA, 253 bp, Proteobacteria sp.; identical to SEQ ID NO: 332

SEQ ID NO: 377 - DNA, 253 bp, Proteobacteria sp.
tacggagggtgcaagcgttaatcggaattactgggcgtaaagcgcacgcaggcggtttgttaagtcagatgtgaaatccccgggctcaacctgggaactgcatttgagactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 378 - DNA, 253 bp, Proteobacteria sp.; identical to SEQ ID NO: 334

SEQ ID NO: 379 - DNA, 253 bp, Proteobacteria sp.; identical to SEQ ID NO: 335

SEQ ID NO: 380 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctagagtctcgtagaggggggtggaattccaggtgtagcggtgaaatgcgtagagatctggaggaataccggtggcgaaggcggccccctggacgaagactgacgctcaggtgcgaaagcgtggggagcaaacagg

SEQ ID NO: 381 - DNA, 253 bp, Proteobacteria sp.
tacggagggtgcaagcgttaatcggaattactgggcgtaaagcgcacgcaggcggtctgttaagtcagatgtgaaatccccgggctcaacctgggaactgcatttgaaactggcaggctagagtcttgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 382 - DNA, 253 bp, Proteobacteria sp.; identical to SEQ ID NO: 338

SEQ ID NO: 383 - DNA, 253 bp, Proteobacteria sp.; identical to SEQ ID NO: 339

SEQ ID NO: 384 - DNA, 253 bp, Proteobacteria sp.; identical to SEQ ID NO: 340

SEQ ID NO: 385 - DNA, 253 bp, Proteobacteria sp.
tacggagggtgcaagcgttaatcggaattactgggcgtaaagcgcacgcaggcggtctgtcaagtcggatgtgaaatccccgggctcaacctgggaactgcatccgatactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 386 - DNA, 253 bp, Proteobacteria sp.
tacggagggtgcaagcgttaatcggaattactgggcgtaaagcgcacgcaggcggtctgtcaagtcagatgtgaaatccccgggctcaacctgggaactgcatttgaaactggcagactggagtcttgtagaggggggtagaattccaagtgtagcggtgaaatgcgtagagatctggaggaataccggtggcgaaggcggccccctggacaaagactgacgctcaggtgcgaaagcgtggggagcaaacagg

SEQ ID NO: 387 - DNA, 253 bp, Proteobacteria sp.
tacggagggtgcaagcgttaatcggaattactgggcgtaaagcgcacgcaggcggtctgtcaagtcggatgtgaaatccccgggctcaacccgggaactgcattcgaaactggcaggctagagtctcgtagaggggggtagaattccaggtgtagcggtgaaatgcgtagagatctggaggaataccagtggcgaaggcggccccctggacgaaaactgacgctcaggtgcgaaagcgtggggagcaaacagg

SEQ ID NO: 388 - DNA, 253 bp, Proteobacteria sp.
tacggagggtgcaagcgttaatcggaattactgggcgtaaagcgcacgcaggcggtctgtcaagtcggatgttaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 389 - DNA, 253 bp, Proteobacteria sp.
  misc_feature (3)..(3): n is a, c, g, or t
tanggagggtgcaagcgttaatcggaattactgggcgtaaagcgcacgcaggcggtctgtcaagtcggatgtgaaatccccgggctcaacctgggaactgcatccgaaactggcaggctagagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 390 - DNA, 254 bp, Proteobacteria sp.
tacggagggtgcaagcgttaatcggaattactgggcgtaaagcgcacgcaggcggtttgttaagtcagatgtgaaatccccggggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 391 - DNA, 253 bp, Proteobacteria sp.
tacggagggtgcaagcgttaatcggaattactgggcgtaaagcgcacgcaggcggttgattaagtcagatgtgaaatccccgggcttaacctgggaactgcatttgaaactggtcagcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 392 - DNA, 253 bp, Proteobacteria sp.; identical to SEQ ID NO: 344

SEQ ID NO: 393 - DNA, 251 bp, Proteobacteria sp.
tacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 394 - DNA, 251 bp, Proteobacteria sp.
tacagaggtctcaagcgttgttcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 395 - DNA, 120 bp, Proteobacteria sp.
tggggaatattgcacaatgggcgcaagcctgatgcagccatgccgcgtgtgtgaagaaggccttcgggttgtaaagcactttcagcggggaggaaggcgttaaggttaataaccttggcg

SEQ ID NO: 396 - DNA, 120 bp, Proteobacteria sp.
tggggaatattgcacaatgggcgcaagcctgatgcagccatgccgcgtgtatgaagaaggccttcgggttgtaaagtactttcagcggggaggaaggcgataaggttaataaccttgtcg

SEQ ID NO: 397 - DNA, 120 bp, Proteobacteria sp.
tggggaatattgcacaatgggcgcaagcctgatgcagccatgccgcgtgtatgaagaaggccttcgggttgtaaagtactttcagcggggaggaaggtgttgtggttaataaccgcagca

SEQ ID NO: 398 - DNA, 120 bp, Proteobacteria sp.
tggggaatattgcacaatgggcgcaagcctgatgcagccatgccgcgtgtatgaagaaggccctcgggttgtaaagtactttcagtcgggaggaaggtggtaaggttaataaccttatca

SEQ ID NO: 399 - DNA, 90 bp, Proteobacteria sp.
tggggaatattgcacaatgggcgcaagcctgatgcagccatgccgcgtgtatgaagaaggccttcgggttgtaaagtactttcagcggga

SEQ ID NO: 400 - DNA, 163 bp, Proteobacteria sp.
tggggaatattgcacaatgggcgcaagcctgatgcagccatgccgcgtgtatgaagaaggccttagggttgtaaagtacttttcagcggggaggaagggagtaaagttaatacctttgctcattgacgttacccgcagaagaagcaccggctaactccgtgcc

SEQ ID NO: 401 - DNA, 99 bp, Proteobacteria sp.
tggggaatattgcacaatgggcgcaagcctgatgcagccatgccgcgtgtatgaagaaggccttcgggttgtaaagtactttctagcggggaggaaggg

SEQ ID NO: 402 - DNA, 96 bp, Proteobacteria sp.
tggggaatattgcacaatgggcgcaagcctgatgcagccatgccgcgtgtatgaagaaggccttcggttgtaagtactttcagcgggaggaaggga

SEQ ID NO: 403 - DNA, 111 bp, Proteobacteria sp.
tggggaatattgcacaatgggcgcaagcctgatgcagccatgccgcgtgtatgaagaaggccttcgggttgtaaagtactttcagcgaggaggaaggcattgtggttaata

SEQ ID NO: 404 - DNA, 111 bp, Proteobacteria sp.
tgggggaatattgcacaatgggcgcaagcctgatgcagccatgccgcgtgtgtgaagaaggccttcgggttgtaaagcactttcagcggggaggaaggcattaaggttaat

SEQ ID NO: 405 - DNA, 99 bp, Proteobacteria sp.
ttcgggttagtaaagttacttcaggcgggaggaagggagtaaagttaatacctttgctcattgacgttacccgcagaagaagcaccggctaactccgtg

SEQ ID NO: 406 - DNA, 99 bp, Proteobacteria sp.
ttcgggttagtaaagttacttcagcggggaggaagggagtaaagttaatacctttactcattgacgttacccgcagaagaagcaccggctaactccgtg

SEQ ID NO: 407 - DNA, 81 bp, Proteobacteria sp.
ttcgaggcgggaggaagggagtaaagttaatacctttgctcattgacgttacccgcagaagaagcaccggctaactccgtg

SEQ ID NO: 408 - DNA, 111 bp, Proteobacteria sp.
tggggaatattgcacaatgggcgcaagcctgatgcagccatgccgcgtgtgtgaagaaggccttcgggttgtaaagcactttcagcggggaggaaggcggtgaggttaata

SEQ ID NO: 409 - DNA, 111 bp, Proteobacteria sp.
tgggggaaatattgcacaatgggcgcaagcctgatgcagccatgccgcgtgtgtgaagaaggccttcgggttgtaaagcactttcagcggggaggaaggcgataaggttaa

SEQ ID NO: 410 - DNA, 99 bp, Proteobacteria sp.
ttcgggttagtaaagctacttcagcggggaggaaggcgataaggttaataaccttgtcgattgacgttacccgcagaagaagcaccggctaactccgtg

SEQ ID NO: 411 - DNA, 83 bp, Proteobacteria sp.
actttcagcggggaggaaggcggtgaggttaataacctcatcgattgacgttacccgcagaagaagcaccggctaactccgtg

SEQ ID NO: 412 - DNA, 111 bp, Proteobacteria sp.
tggggaatattgcacaatggcgcaagcctgatgcagccatgccgcgtgtatgaagaaggccttcgggttgtaaagtactttcagcgaggaggaaggggatgtggttaataa

SEQ ID NO: 413 - DNA, 111 bp, Proteobacteria sp.
tggggaatattgcacaatgggcgcaagcctgatgcagccatgccgcgtgtatgaagaaggccttcgggttgtaaagtactttcagcggggaggaaggcgataaggttaata

SEQ ID NO: 414 - DNA, 102 bp, Proteobacteria sp.
ccgttcgggttagtaaagctacttcagcggggaggaaggcggtgaggttaataacctcaccgattgacgttacccgcagaagaagcaccggctaactccgtg

SEQ ID NO: 415 - DNA, 89 bp, Proteobacteria sp.
agtaagctacttcagcgggaggaaggcggtgaggttaataacctcaccgattgacgttacccgcagaagaagcaccggctaactccgtg

SEQ ID NO: 416 - DNA, 83 bp, Proteobacteria sp.
actttcagcggggaggaaggcgttaaggttaataaccttggcgattgacgttacccgcagaagaagcaccggctaactccgtg

SEQ ID NO: 417 - DNA, 102 bp, Proteobacteria sp.
ccgttcgggttagtaaagtactttcagcgaggaggaaggcattgtggttaataaccgcagtgattgacgttactcgcagaagaagcaccggctaactccgtg

SEQ ID NO: 418 - DNA, 82 bp, Proteobacteria sp.
cttcgaggcgggaggaaggcgataaggttaataaccttgtcgattgacgttacccgcagaagaagcaccggctaactccgtg

SEQ ID NO: 419 - DNA, 111 bp, Proteobacteria sp.
tgggggaatattgcacaatgggcgcaagcctgatgcagccatgccgcgtgtatgaagaaggccttcgggttgtaaagtactttcagcggggaggaaggtgttgaggttaat

SEQ ID NO: 420 - DNA, 363 bp, Proteobacteria sp.
  misc_feature (2)..(2): n is a, c, g, or t
cntacgggtggctgcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcagggatgctggaattcgtggtgtagcggtgaaatgcttagatatcacgaagaactccgatcgcgaaggcatgtgtccggagtgcaactgacgctgaggctcgaaagtgtgggtatcaaaca

SEQ ID NO: 421 - DNA, 363 bp, Proteobacteria sp.
cctacgggaggcagcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgttttgctagagtgtcggagaggtaagtggaattcctagtgtagcggtgaaatgcgtag

SEQ ID NO: 422 - DNA, 465 bp, Proteobacteria sp.
cctacgggcggctgcagtggggaatattgcacaatgggcgcaagcctgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaatacggagggtgcaagcgttaatcggaattactgggcgtaaagcgcacgcaggcggtctgtcaagacggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtc

SEQ ID NO: 423 - DNA, 465 bp, Proteobacteria sp.
  misc_feature (2)..(2): n is a, c, g, or t
cntacgggaggcagcagtggggaatattgcacaatgggcgcaagcctgatgcagccatgccgcgtgtatgaagaaggccttcgggttgtaaagtactttcagcgaggaggaaggcgataaggttaataaccttgtcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctagtagtc

SEQ ID NO: 424 - DNA, 465 bp, Proteobacteria sp.
cctacgggaggcagcagtggggaatattgcacaatgggcgcaagcctgatgcagccatgccgcgtgtatgaagaaggccttcgggttgtaaagtactttcagcgaggaggaaggtgttgtgattaataaccgcagcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaagtccaggtgtagcggtgaaatgcgtagagatctggaggaataccggtggcgaaggcggccccctggacgaagactgacgctcaggtgcgaaagcgtggggagcaaacaggattagataccctggtagtc

SEQ ID NO: 425 - DNA, 465 bp, Proteobacteria sp.
cctacgggaggctgcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaaaactgacgctcaggtgcgaaagcgtggggagcaaacaggattagataccccagtagtc

SEQ ID NO: 426 - DNA, 465 bp, Proteobacteria sp.
cctacggggggcagcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccccggtagtc

SEQ ID NO: 427 - DNA, 465 bp, Proteobacteria sp.
cctacgggaggctgcagtggggaatattgcacaatgggcgcaagcctgatgcagccatgccgcgtgtatgaagaaggccttcgggttgtaaagtactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaaccgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtc

SEQ ID NO: 428 - DNA, 464 bp, Proteobacteria sp.
cctacgggagcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagaggtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggactagataccctagtagtc

SEQ ID NO: 429 - DNA, 465 bp, Proteobacteria sp.
cctacgggtggcagcagtggggaatattgtacaatgggcgcaagcctgatgcagccatgccgcgtgtgtgaagaaggccttcgggttgtaaagcactttcagcggggaggaaggcgttaaggttaataaccttggcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggcttaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagatacccttgtagtc

SEQ ID NO: 430 - DNA, 465 bp, Proteobacteria sp.
  misc_feature (2)..(2): n is a, c, g, or t
cntacgggcggctgcagtggggaatattgcacaatgggcgcaagcctgatgcagccatgccgcgtgtatgaagaaggccttcgggttgtaaagtactttcagcggggaggaagggagtgaggttaataaccttattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagatacccttgtagtc

SEQ ID NO: 431 - DNA, 465 bp, Proteobacteria sp.
cctacgggcggctgcagtggggaatattgcacaatgggcgcaagcctgatgcagccatgccgcgtgtatgaagaaggccttcgggttgtaaagtactttcagcgaggaggaaggtgttgtggttaataaccacagcaattgacgttactcgcagaagaagcaccggctaactccgtgccagcagccgcggtaatacggagggtgcaagcgttaatcggaattactgggcgtaaagcacacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtc

SEQ ID NO: 432 - DNA, 465 bp, Proteobacteria sp.
cctacgggaggctgcagtggggaatattgcacaatgggcgcaagcctgatgcagccatgccgcgtgtatgaagaaggccttcgggttgtaaagtactttcagcgaggaggaaggcgttaaggttaataaccttagtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagatacccgagtagtc

SEQ ID NO: 433 - DNA, 465 bp, Proteobacteria sp.
cctacgggaggcagcagtggggaatattgcacaatgggcgcaagcctgatgcagccatgccgcgtgtatgaagaaggccttcgggttgtaaagtactttcagcgaggaggaaggtgttgtggttaataactacagcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtc

SEQ ID NO: 434 - DNA, 464 bp, Proteobacteria sp.
cctacgggggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagatacccctgtagtc

SEQ ID NO: 435 - DNA, 465 bp, Proteobacteria sp.
cctacggggggctgcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagatacccttgtagtc

SEQ ID NO: 436 - DNA, 465 bp, Proteobacteria sp.
cctacgggaggcagcagtggggaatattgcacaatgggcgcaagcctgatgcagccatgccgcgtgtatgaagaaggccttcgggttgtaaagtactttcagcgaggaggaaggtgttgaggttaataaccgcagcaattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccccggtagtc

SEQ ID NO: 437 - DNA, 465 bp, Proteobacteria sp.
tctacgggaggctgcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccccagtagtc

SEQ ID NO: 438 - DNA, 465 bp, Proteobacteria sp.
cctacgggaggctgcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaatacggagggtgcaagcgttaatcggaattactgggcgtaaagcgcacgcaggcggtctgttaagtcagatgtgaaatccccgggctcaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtc

SEQ ID NO: 439 - DNA, 463 bp, Proteobacteria sp.
ctacgggggctgcagtggggaatattgcacaatgggcgcaagcctgatgcagccatgccgcgtgtgtgaagaaggccttcgggttgtaaagcactttcagcggggaggaaggcgttaaggttaataaccttggcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtc

SEQ ID NO: 440 - DNA, 464 bp, Proteobacteria sp.
cctacgggtggctgcagtggggaatattgcacaatgggcgcaagcctgatgcagccatgccgcgtgtatgaagaaggccttcgggttgtaaagtactttcagcgaggaggaaggtgttgaggttaataacctcagcaattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagatacccggtagtc

SEQ ID NO: 441 - DNA, 465 bp, Proteobacteria sp.
cctacgggaggcagcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtc

SEQ ID NO: 442 - DNA, 465 bp, Proteobacteria sp.
cctacgggcggctgcagtggggaatattgcacaatgggcgcaagcctgatgcagccatgccgcgtgtatgaagaaggccttcgggttgtaaagtactttcagcgaggaggaaggtgttgtggttaataaccgcagcaattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctagtagtc

SEQ ID NO: 443 - DNA, 465 bp, Proteobacteria sp.
cctacgggtggctgcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccggggcaaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtc

SEQ ID NO: 444 - DNA, 464 bp, Proteobacteria sp.
cctacggggggctgcagtggggaatattgcacaatgggcgcaagcctgatgcagccatgccgcgtgtatgaagaaggccttcgggttgtaaagtactttcagcggggaggaaggcgataaggttaataaccttgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctgtagtc

SEQ ID NO: 445 - DNA, 465 bp, Proteobacteria sp.
cctacgggcggctgcagtggggaatattgcacaatgggcgcaagcctgatgcagccatgccgcgtgtatgaagaaggccttcgggttgtaaagtactttcagcgaggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctagtagtc

SEQ ID NO: 446 - DNA, 363 bp, Proteobacteria sp.
cctacgggcggcagcagtggggaatattgcacaatgggcgcaagcctgatgcagccatgccgcgtgtatgaagaaggccttcgggttgtaaagtactttcagcggggaggaaggcgataaggttaataaccttgtcgattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtag

SEQ ID NO: 447 - DNA, 363 bp, Proteobacteria sp.
cctacgggtggctgcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtag

SEQ ID NO: 448 - DNA, 298 bp, Proteobacteria sp.
  misc_feature (2)..(2): n is a, c, g, or t
cntacgggtggctgcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtc

SEQ ID NO: 449 - DNA, 363 bp, Proteobacteria sp.
  misc_feature (2)..(2): n is a, c, g, or t
cntacgggtggctgcagtggggaatattgcacaatgggcgcaagcctgatgcagccatgccgcgtgtatgaataaggatcggctaactccgtgccagcagccgcggtaatacggagggtgctagcgttaatcggaattactgggcgtaaagcgcacgcaggcggtttgttaagtcagatgtgaaatccccgggctcaacctgggaactgcatctgatactggcaagcttgagtctcgtagaggtaagcggaattcctggtgtagcggtgaaatgcgtagagatcaggaggaacatcggtggcgaaggcggcttactgggcttttactgacgctgaggctcgaaagcgtggggagcaaacagga

SEQ ID NO: 450 - DNA, 248 bp, Proteobacteria sp.
ctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 451 - DNA, 191 bp, Proteobacteria sp.
tgagacacggccagactcctacgggaggcagcagtggggaatattgcacaatgggcgcaagcctgatgcagccatgccgcgtgtatgaagaaggccttcgggttgtaaagtactttcagcgaggaggaaggcgttaaggttaataaccttagcgattgacgttactcgcagaagaagcaccggctaactcc

SEQ ID NO: 452 - DNA, 257 bp, Proteobacteria sp.
aacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataatgtcgcaagaccaaagtgggggaccttcgggcctcacaccatcggatgtgcccagatgggattagctagtaggtggggtaacggctcacctaggcgacgatcccctag

SEQ ID NO: 453 - DNA, 473 bp, Proteobacteria sp.
caggcctaacacatgcaagtcgaacggtaacagaaatcagctttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 454 - DNA, 484 bp, Proteobacteria sp.
aacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatcccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagcactttcagcggggaggaaggcggtgaggttaataacctcatcgattgacgttacccgcagaagaagcaccggctaactcc

SEQ ID NO: 455 - DNA, 495 bp, Proteobacteria sp.
tggctcagattgaacgctgcggcaggcctaacacatgcaagtcgaacggtaacaggaagaagcttgcttcgtttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 456 - DNA, 479 bp, Proteobacteria sp.
ctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgccccagatggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 457 - DNA, 468 bp, Proteobacteria sp.
caggcctaacacatgcaagtcgagcggtagcacagagagcttgctctcgggtgacgagcggcgacgggtgagtaatgtctgggaaactgcctgatgagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgttaaggttaataaccttggcgattgacgttacccgcagaagaagcaccggctaactcc

SEQ ID NO: 458 - DNA, 483 bp, Proteobacteria sp.
gattgaacgctggcggcaggcctaacacatgcaagtcgaacggtaacagaatcagcttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatggattagctagtaggtgggtaacggctcacctaggcgacgatccctagctggtctgagaggatgaccagccacactggaactgagacacggtccagactcctacggaggcagcagtgggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 459 - DNA, 310 bp, Proteobacteria sp.
accttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 460 - DNA, 475 bp, Proteobacteria sp.
gcaggcctaacacgatgcaagtcgaacggtaacaggaagagcttgcttcgttgctgacgagtggcggacgggtgagtaatgtctgggaaactgcctgatggagggggataactactggaaacggtagctaataccgcagtaacgtcgcaagaccaaagaggggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 461 - DNA, 209 bp, Proteobacteria sp.
ccagccacactggaactgagacacggtccagactcctacgggaggcagcagtggggaatattgcacaatgggcgcaagcctgatgcagccatgccgcgtgtatgaagaaggccttcgggttgtaaagtacttttcagcggggaggaagggagtaaagttaatacctttgctcattgacgttacccgcagaagaagcaccggctaactcc

SEQ ID NO: 462 - DNA, 319 bp, Proteobacteria sp.
ccaaagagggggaccttcgggcctcttgccatcggatgtgcccagatgggattagctgttggtgggtaacggctcaccaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 463 - DNA, 310 bp, Proteobacteria sp.
accctcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 464 - DNA, 486 bp, Proteobacteria sp.
agattgaacgctggcggcaggcctaacacatgcaagtcgaacggtaacaggaatcagcttgctgatttgctgacgagtggcggacgggtgagtaatgtctgggaaactgcctgatgagggggataactactggaaacggtagctaataccgcataacgtcgcaagaccaaagagggggaccttcgggcctcttgccatcggatgtgcccagatggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 465 - DNA, 331 bp, Proteobacteria sp.
tcgcaagaccaaagagggggaccttcgggcctcttgccatcagatgtgcccagatgggattagctagtaggtggggtaacggctcacctaggcaacgatcccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgttgaggttaataacctcagcgattgacgttacccgcagaagaagcaccggctaactcc

SEQ ID NO: 466 - DNA, 485 bp, Proteobacteria sp.
attgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagaggggggaccttcgggcctcttgccatcagatgtgcccagatggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataaccttgtcgattgacgttacccgcagaagaagcaccggctaactcc

SEQ ID NO: 467 - DNA, 291 bp, Proteobacteria sp.
ccatcggatgtgcccagatgggattagcttgttgtggggtaacggctcaccaaggcgacgatccctagctggtctgagaggatgaccagccacactggaactgagacacggtccagactcctacggaggcagcatggggaatattgcacaatgggcgcaagcctgatgcagccatgccgcgtgtatgaagaaggccttcgggttgtaaagtactttcagcggggaggaagggagtaaagttaatacctttactcattgacgttacccgcagaagaagcaccggctaactcc

SEQ ID NO: 468 - DNA, 490 bp, Proteobacteria sp.
ctcagattgaacgctggcggcaggcctaacacatgcaagtcgaacggtaacaggaaacagcttgctgtttcgctgacgagtggcggacgggtgagtaatgtctgggaaactgcctgatgagggggataactactggaaacggtagctaataccgcataacgtcgcaagaccaaagagggggaccttcgggcctcttgccatcggatgtgcccagatggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactcc

SEQ ID NO: 469 - DNA, 249 bp, Proteobacteria sp.
attgaacgctggcggcaggcctaacacatgcaagtcgaacggtaacaggaagcagcttgctgctttgctgacgagtggcggacgggtgagtaatgtctgggaaactgcctgatggaggggataactactggaaacggtagctaataccgcataacgtcgcaagaccaaagagggggaccttagggcctcttgccatcggatgtgcccagatgggattagctagtaggtgggtaaaggctcacctaggcg

SEQ ID NO: 470 - DNA, 249 bp, Proteobacteria sp.
ctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtagggtggggtaatggctcacctaggcgacgatcc

SEQ ID NO: 471 - DNA, 249 bp, Proteobacteria sp.
attgaacgctggcggcaggcctaacacatgcaagtcgaacggtaacaggaaacagcttgctgtttcgctgacgagtggcggacgggtgagtaatgtctgggaagctgcctgatggagggggataactactggaaacggtagctaataccgcataatgtcgcaagaccaaagagggggaccttcgggcctcttgccatcggatgtgcccagatgggattagctagtaggtgggtaacggctcacctaggc

SEQ ID NO: 472 - DNA, 151 bp, Proteobacteria sp.
tagggcggcaagacctcgatgcagccactgctcgcgtgtatgaagaaggccttcgggttgtaaagtactttcagcggggaggaagggagtaaagttaatacctttgctcattgacgttacccgcagaagaagcaccggctaactccgtgcc

SEQ ID NO: 473 - DNA, 249 bp, Proteobacteria sp.
cggcaggcctaacacatgcaagtcgaacggtacagaagcagcttgctgctttgctgacgagtggcgacgggtgagtaatgtctgggaaactgcctgatggagggggataactactggaaacggtagctaataccgcataatgtcgcaagaccaaagagggggaccttcgggcctcttgccatcggatgtgcccagatgggattagctagtaggtgggtaaaggctcacctaggcgacgatccctagctg

SEQ ID NO: 474 - DNA, 249 bp, Proteobacteria sp.
attgaacgctggcggcaggcctaacacatgcaagtcgaacggtaacagaaagcagcttgctgctttgctgacgagtggcggacgggtgagtaatgtctgggaaactgcccgagtgaggggggataactactggaaacggtagctaataccgcataacgtcgcaagaccaaagagggggaccttagggccttttgccatcggatgtgcccagatgggattagctagtaggtgggtaacggctcacctagg

SEQ ID NO: 475 - DNA, 249 bp, Proteobacteria sp.
attgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtgggtaacggctcacctaggcga

SEQ ID NO: 476 - DNA, 437 bp, Proteobacteria sp.
cagcttgctgattcgctggacgagtggcggacgggtgagtaatgtctgggaaactgcctgatgagggggatactactggaacggtagctaataccgcataacgtcgcaagaccaaagagggggaccttcgggcctcttgccatcggatgtgcccagatgggattagctagtaggtgggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 477 - DNA, 249 bp, Proteobacteria sp.
ctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccg

SEQ ID NO: 478 - DNA, 340 bp, Proteobacteria sp.
ctaatacgtcgcaagaccaaagagggggaccttgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggcaactccgtgcc

SEQ ID NO: 479 - DNA, 491 bp, Proteobacteria sp.
attgaacgctggcggcaggcctaacacatgcaagtcgaacggtagcacagaggagcttgctcttgggtgacgagtggcggacgggtgagtaatgtctgggaaactgcccgatgagggggataactactggaaacggtagctaataccgcataacgtcgcaagaccaaagagggggaccttcgggcctcttgccatcggatgtgcccagatgggattagctagtaggtgggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggtgttgtggttaataaccgcagcaattgacgttactcgcagaagaagcaccggctaactccgtgcc

SEQ ID NO: 480 - DNA, 475 bp, Proteobacteria sp.
gcaggcctaacacatgcaagtcgagcggtagcacagagagcttgctctcgggtgacgagcggcgacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgcc

SEQ ID NO: 481 - DNA, 490 bp, Proteobacteria sp.
attgaacgctggcggcaggcctaacacatgcaagtcgaacgggtaacaggaacagcttgctgtttcgctgacgagtggcggacgggtgagtaatgtctgggaaactgcctgatggaggggataactactggaaacggtagctaataccgcataacgtcgcaagaccaaagagggggaccttcgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 482 - DNA, 431 bp, Proteobacteria sp.
tgctgctttgctgacgagtggcggacgggtgagtaatgtctgggaaactgcctgatggaggggataactactggaaacggtagctaataccgcataacgtcgcaagaccaaagagggggaccttcgggcctcttgccatcagatgtgcccagatgggattagctagttggtgggtaacggctcaccaaggcgacgatccctagctggtctgagaggatgaccagccacactggaactgagacacggtccagactcctacgggaggcagcagtgggaatattgcacaatgggcgcaagcctgatgcagccatgccgcgtgtatgaagaaggccttcgggttgtaaagtactttcagcgggaggaaggtgttgtggttaataaccgcagcaattgacgttacccgcagaagaagcaccggctaactccgtgcc

SEQ ID NO: 483 - DNA, 485 bp, Proteobacteria sp.
ttgaacgctggcggcaggcctaacacatgcaagtcgaacggtacaggaacagcttgctgtttgctgacgagtggcggacgggtgagtaatgtctgggaaactgcctgatggaggggataactactggaaacggtagctaataccgcataacgtcgcaagaccaaagagggggaccttcgggcctcttgccatcggatgtgcccagatggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 484 - DNA, 229 bp, Proteobacteria sp.
tggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataacctcatcgattgacgttacccgcagaagaagcaccggctaactccgtgcc

SEQ ID NO: 485 - DNA, 503 bp, Proteobacteria sp.
ttatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactaagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttactcattgacgttacccgcagaagaagcaccggctaactccgtgcc

SEQ ID NO: 486 - DNA, 464 bp, Proteobacteria sp.
acacatgcaagtcgaacggtaacaggaagcagcttgctgcttcgctgacgagtggcggacgggtgagtaatgtctgggaaactgcctgatggaggggataactactggaaacggtagctaataccgcataacgtcgcaagaccaagaggggaccttcggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 487 - DNA, 486 bp, Proteobacteria sp.
ctggcggcaggcctaacacatggcaagtcgaacggtaacagaaagcagcttgctgctttgctgacgagtggcggacgggtgagtaatgtctgggaaactgcctgatggagggggagtaactactggaaacggtagctaataccgcataacgtcgcgagaccaaagagggggaccttcgggcctcttgccatcggatgtgcccagatgggattagctagtaggtgggtaaagggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 488 - DNA, 363 bp, Proteobacteria sp.
tactggaaacggtagctaataccgcataacgtcgcaagaccaaatgggggaccttcgggcctcatgccatcagatgtgccagatgggattagctagtaggtgggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgttaaggttaataaccttggcgattgacgttacccgcagaagaagcaccggctaactccgtgcc

SEQ ID NO: 489 - DNA, 446 bp, Proteobacteria sp.
acaggaatcagcttgctgattcgctgacgagtggcggacgggtgagtaatgtctgggaaactgcctgatggagggggataactactggaacggtagctaataccgcataacgtcgcaagaccaaagagggggaccttcgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 490 - DNA, 340 bp, Proteobacteria sp.
ataacgtcgcaagaccaaagagggggaccttcgggcctcttgccatcagatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 491 - DNA, 249 bp, Proteobacteria sp.
attgaacgctggcggcaggcctaacacatgcaagtcgaacggtaacagaaagcagcttgctgctttgctgacgagtggcggacgggtgagtaatgtctgggaaactgcctgatggagggggataactactggaaacggtagctaataccgcataacgtcgcaagaccaaagagggggaccttcggacctcttgccatcggatgtgcccagatgggattagcttgttggtgggtaacggctcaccaaggc

SEQ ID NO: 492 - DNA, 486 bp, Proteobacteria sp.
aacgctggcggcaggcctaacacatgcaagtcgaacggtaacaggaagaagcttgcttcttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 493 - DNA, 211 bp, Proteobacteria sp.
cagccacactggaactgagacacggccagactcctacgggaggcagcagtggggaatattgcacaatgggcgcaagcctgatgcagccatgccgcgtgtatgaagaaggccttcgggttgtaaagtactttcagtcgggaggaaggtgttaaggttaataaccttgacaattgacgttaccgacagaagaagcaccggctaactccgtgcc

SEQ ID NO: 494 - DNA, 225 bp, Proteobacteria sp.
tgagaggatgaccagccacactggaactgagacacggtccagactcctacgggaggcagcagtggggaatattgcacaatgggcgcaagcctgatgcagccatgccgcgtgtgtgaagaaggccttcgggttgtaaagctactttcagcggggaggaaggcgataaggttaataaccttgtcgattgacgttacccgcagaagaagcaccggctaactccgtgcc

SEQ ID NO: 495 - DNA, 474 bp, Proteobacteria sp.
cggcaggcctaacacatgcaagtcgaacggtaacaggagaagcttgctctttgctgacgagtggcggacggtgagtaatgtctgggaaactgcctgatggaggggataactactggaaacggtagctaataccgcataacgtcgcaagaccaaagagggggaccttcgggcctcttgccatcggatgtgcccagatggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 496 - DNA, 163 bp, Proteobacteria sp.
tggggaatattgcacaatgggcgcaagcctgatgcagccatgccgcgtgtatgaagaaggccttcgggttgtaaagtacttttcagcggggaggaagggagtaaagttaatacctttgctcattgacgttacccgcagaagaagcaccggctaactccgtgcc

SEQ ID NO: 497 - DNA, 203 bp, Proteobacteria sp.
actggaactgagacacggtccagactctacggaggcagcagtggggaatattgcacaatgggcgcaagcctgatgcagccatgccgcgtgtatgaagaaggccttcgggctgtaaagtactttcagcggggaggaagggagtaaagttaatacctttgctcattgacgttacccgcagaagaagcaccggctaactccgtgcc

SEQ ID NO: 498 - DNA, 331 bp, Proteobacteria sp.
cgcaagaccaaagagggggaccttcgggcctcttgccatcggatgtgcccagatgggattagctgttggtgggtaacggctcaccaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 499 - DNA, 446 bp, Proteobacteria sp.
aacaggaagcagcttgctgcttcgctgacgagtggcggacgggtgagtaatgtctgggaaactgcctgatggaggggataactactggaaacggtagctaataccgcataacgtcgaaagaccaaagaggggaccttcgggcctcttgccatcggatgtgcccagatgggattagcttgttggtgggtaatggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 500 - DNA, 491 bp, Proteobacteria sp.
ttgaacgctggcggcaggcctaacacatgcaagtcgagcggtaacacagggagcttgctcctgggtgacggagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtgggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgcc

SEQ ID NO: 501 - DNA, 492 bp, Proteobacteria sp.
attgaacgctggcggcaggcctaacacatgcaagtcgaacggtaacaggaaacagcttgctggttttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 502 - DNA, 481 bp, Proteobacteria sp.
ctggcggcaggcctaacacatgcaagtcgaacggtaacgaggaagcagcttgctgcttcgctgacgagtggcggacgggtgagtaatgtctgggaaactgcctgatggaggggataactactggaaacggtggctaataccgcataacgtcgcaagaccaaagaggggaccttcgggcctcttgccatcagatgtgcccagatggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataaccttgtcgattgacgttacccgcagaagaagcaccggctaactccgtgcc

SEQ ID NO: 503 - DNA, 489 bp, Proteobacteria sp.
attgaacgctggcggcaggcctaacacatgcaagtcgacggtaacagaaagcagcttgctgctttgctgacgagtggcggacgggtgagtaatgtctgggaaactgcctgatggaggggataactactggaaacggtagctaataccgcataacgtcgcaagaccaaagagggggaccctcgggcctcttgccatcggatgtgcccagatgggattagcttgttggtgggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 504 - DNA, 492 bp, Proteobacteria sp.
agattgaacgctggcggcaggcctaacacatgcaagtcgaacggtaacaggaagcagcttgctgctttgctgacgagtggcggacgggtgagtaatgtctgggaaactgcctgatggaggggataactactggaaacggtagctaataccgcataacgtcgcaagaccaaagagggggaccttagggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 505 - DNA, 429 bp, Proteobacteria sp.
gctctcgggtgacgagcggcggacgggtgagtaatgtctgggaaactgctgatgagggggataactactggaaacggtagctaataccgcataatgtcgcaagacaaagtgggggaccttcgggcctcatgccatcagatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgttaaggttaataaccttggcgattgacgttacccgcagaagaagcaccggctaactccgtgcc

SEQ ID NO: 506 - DNA, 444 bp, Proteobacteria sp.
gaagaagcttgcttcttgctgacgagtggcggacgggtgagtaatgtctgggaaactgcctgatggagggggataactactggaaacggtagctaataccgcataacgtcgcaagaaccaaagagggggaccttcgggcctcttgccatcggatgtgcccagatgggatttagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 507 - DNA, 478 bp, Proteobacteria sp.
ctggcggcaggcctaacacatgcaagtcgaacggtaacagaaagcagcttgctgctttgctgacgagtggcggacggtgagtaatgtctgggaactgcctgatggaggggataactactggaaacggtagctaataccgcataacgtcgcaagaccaaagaggggaccttagggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgggaggaagggagtaaagttaatacctttgctcattgacgttacccgcagaagaagcaccggctaactccgtgcc

SEQ ID NO: 508 - DNA, 436 bp, Proteobacteria sp.
agcttgctgcttcgctgacgagtggcggacgggtgagtaatgtctgggaaactgcctgatggaggggagtaactactggaaacggtagctaataccgcataacgtcgcaagaccaaagagggggaccttcgggcctcttgccatcggatgtgcccagatggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgggaggaagggagtaaagttaatacctttgctcattgacgttacccgcagaagaagcaccggctaactccgtgcc

SEQ ID NO: 509 - DNA, 437 bp, Proteobacteria sp.
agcttgctgttttgctgacgagtggcggacggtgagtaatgtctgggaaactgcctgatggaggggagtaactactggaaacggtagctaataccgcataacgtcgcaagaccaaagagggggaccttcgggcctcttgccatcggatgtgcccagatgggattagctagtaggtgggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 510 - DNA, 421 bp, Proteobacteria sp.
ctgacgagtgcggacgggtgagtaatgtctgggaaactgcctgatggaggggataactactggaaacggtagctaataccgcataacgtcgcaagaccaaagagggggaccttcgggcctcttgccatcggatgtgcccagatggattagctagtaggtgggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 511 - DNA, 192 bp, Proteobacteria sp.
tgagacacggtccagactcctacggaggcagcagtgggaatattgcacaatgggcgcaagcctgatgcagccatgccgcgtgtatgaagaagccttcggttgtaaagtactttcagcgggaggaagggagtaaagttaatacctttgctcattgacgttacccgcagaagaagcaccggctaactccgtgcc

SEQ ID NO: 512 - DNA, 351 bp, Proteobacteria sp.
tagctaataccgcataacgtcgcagaccaaagaggggaccttcgggcctcttgccatcggatgtgcccagatgggattagctagtaggtgggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 513 - DNA, 490 bp, Proteobacteria sp.
attgaacgctggcggcaggcctaacacatgcaagtcgaacggtaacaggaaacagcttgctgtttcgctgacgagtggcggacgggtgagtaatgtctgggaaactgcctgatgagggggataactactggaaacggtagctaataccgcataacgtcgcaagaccaaagagggggaccttcgggcctcttgccatcggatgtgcccagatgggattagcttgttggtgggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgcc

SEQ ID NO: 514 - DNA, 177 bp, Akkermansia muciniphila
tatgcgccttgccagcccgctcaggtgtgccagcagccgcggtaatacagaggtctcaagcgttgttcggaatcactgggcgtaaagcgtgcgtaggctgtttcgtaagtcgtgtgtgaaaaggcgcgggctcaacccgcggacggcacatgatactgcgagactagagtaatggag

SEQ ID NO: 515 - DNA, 112 bp, Akkermansia muciniphila
ggaattctcggtgtagcagtgaaatgcgtagatatcgagaggaacactcgtggcgaaggcgggttcctggacattaactgacgctgaggcacgaagccagggagcgaaaggg

SEQ ID NO: 516 - DNA, 117 bp, Akkermansia muciniphila
aaccggaattctcggtgtagcagtgaaatgcgtagatatcgagaggaacactcgtggcgaaggcgggttcctggacattaactgacgctgaggcacgaagccaggggagcgaaaggg

SEQ ID NO: 517 - DNA, 177 bp, Akkermansia muciniphila
tatgcgccttgccagcccgctcaggtgtgccagcagccgcggtaatacagaggtctcaagcgttgttcggaatcactgggcgtaaagcgtgcgtaggctgtttcgtaagtcgtgtgtgaaaggcaggggctcaacccctggattgcacatgatactgcgagactagagtaatggagg

SEQ ID NO: 518 - DNA, 122 bp, Akkermansia muciniphila
aggggaaccgaattctcggtgtagcagtgaaatgcgtagatatcgagaggaacactcgtggcgaaggcgggttcctggacattaactgacgctgaggcacgaaggccaggggagcgaaaggg

SEQ ID NO: 519 - DNA, 252 bp, Akkermansia muciniphila
tacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagg

SEQ ID NO: 520 - DNA, 252 bp, Akkermansia muciniphila
tccctctttctcaatctttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagg

SEQ ID NO: 521 - DNA, 253 bp, Akkermansia muciniphila
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 522 - DNA, 252 bp, Akkermansia muciniphila
tacgtagggggcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagg

SEQ ID NO: 523 - DNA, 252 bp, Akkermansia muciniphila
aacgtaggtcacaagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagg

SEQ ID NO: 524 - DNA, 251 bp, Akkermansia muciniphila
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatatggaggaacaccggtggcgaaagcggctctctggtctgtaactgacgctgaggcacgaaggccaggggagcgaaag

SEQ ID NO: 525 - DNA, 251 bp, Akkermansia muciniphila
tacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatattaggaggaacaccagtggcgaaggcgggttcctggacattaactgacgctgaggcacgaaggccaggggagcgaaag

SEQ ID NO: 526 - DNA, 251 bp, Akkermansia muciniphila
aacgtagggtgcaagcgttgtccggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaag

SEQ ID NO: 527 - DNA, 251 bp, Akkermansia muciniphila
tacagaggtctcaagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcgggaggaacaccagtggcgaaggcggcttcctggcacacaactgacgctgaggcacgaaggccaggggagcgaaag

SEQ ID NO: 528 - DNA, 251 bp, Akkermansia muciniphila
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggaggggggtagaattccaggtgtagcggtgaaatgcgtagatatcgagaggaacactcgtggcgaaggcgggttcctggacattaactgacgctgaggcacgaaggccaggggagcgaaaggg

SEQ ID NO: 529 - DNA, 251 bp, Akkermansia muciniphila
tacagaggtctcaagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaag

SEQ ID NO: 530 - DNA, 251 bp, Akkermansia muciniphila
tacggagggggctagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcaaaca

SEQ ID NO: 531 - DNA, 251 bp, Akkermansia muciniphila
tacagaggtctcaagcgttgttcggaatcactgggcgtaaagcgtgcgtaggctgtttcgtaagtcgtgtgtgaaaggcaggggctcaacccctggattgcacatgatactgtcggtctagagtatgtgagagggaagtggaattcccggtgtagcggtgaaatgcgtagatatcgagaggaacactcgtggcgaaggcgggttcctggacattaactgacgctgaggcacgaaggccaggggagcgaaag

SEQ ID NO: 532 - DNA, 253 bp, Akkermansia muciniphila; identical to SEQ ID NO: 521

SEQ ID NO: 533 - DNA, 251 bp, Akkermansia muciniphila
tacgtaggtggcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaag

SEQ ID NO: 534 - DNA, 120 bp, Akkermansia muciniphila
tcgagaatcattcacaatgggggaaaccctgatggtgcgacgccgcgtgggggaatgaaggtcttcggattgtaaacccctgtcatgtgggagcaaattaaaaagatagtaccacaagag

SEQ ID NO: 535 - DNA, 298 bp, Akkermansia muciniphila
  misc_feature (8)..(8): n is a, c, g, or t
cctacggncggcagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagataccccagtagtc

SEQ ID NO: 536 - DNA, 363 bp, Akkermansia muciniphila
cctacgggtggctgcagtcgagaatcattcacaatgggggcaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcaggggctcaacccctggattgcacatgatactgcgagactagagtaatggagggggaaccggaattctcggtgtagcagtgaaatgcgtagatatcacgaagaactccga

SEQ ID NO: 537 - DNA, 445 bp, Akkermansia muciniphila
cctacgggggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtc

SEQ ID NO: 538 - DNA, 363 bp, Akkermansia muciniphila
cctacggggggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaggaacaccag

SEQ ID NO: 539 - DNA, 363 bp, Akkermansia muciniphila
cctacgggtggctgcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcg

SEQ ID NO: 540 - DNA, 257 bp, Akkermansia muciniphila
cgtgtgccttggcagtcgactagagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggt

SEQ ID NO: 541 - DNA, 409 bp, Akkermansia muciniphila
aattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactct

SEQ ID NO: 542 - DNA, 249 bp, Akkermansia muciniphila
agagtttgatcatggctcagaacgaacgctggcggcgtggataagacatgcaagtcgaacgggagaattgctagcttgctaataattctctagtggcgcacgggtgagtaacacgtgagtaacctgcctcttagtgggggatagccctgggaaaccgggattaataccgcatacgattgaaagatcaaagcagcaatgcgctaggagatgggctcgcggcctattagttagttggtgaggtaacggctc

SEQ ID NO: 543 - DNA, 249 bp, Akkermansia muciniphila
aacgctggcggcgtggataagacatgcaagtcgaacgggagaattgctagcttgctaataattctctagtggcgcacgggtgagtaacacgtgagtaacctgcccccaagagtgggatagccccgggaaactgggattaataccgcataaaatcgcaagattaaagcagcaatgcgcttggggatgggctcgcgtcctattagttagttggtgaggtaacggctcaccaaggcgatgacgggtagccgg

SEQ ID NO: 544 - DNA, 249 bp, Akkermansia muciniphila
agtttgatcctggctcaagaacgacgctggcggcgtggataagacatgcaagtcgaacgagagaattgctagcttgctaataattctctagtggcgcacggggtgagtaacacgtgagtaacctgcccccgagagcgggatagccctgggaaactgggattaataccgcatagtatcgaaagattaaagcagcaatgcgcttgggatgggctcgcggcctattagttagttggtgaggtaacggctcac

SEQ ID NO: 545 - DNA, 158 bp, Akkermansia muciniphila
ggcagcagtcgaagaatcattcaacaaggtacgggggaaaccctgatggtgcgacgccgcggtgggggaatgaaggtcttcggattgtaaacccctgtcatgtgggagcaaattaaaaagatagtaccacaagaggaagagacggctaactctgtgcc

SEQ ID NO: 546 - DNA, 222 bp, Akkermansia muciniphila
gatgacgggtagccggtctgagaggatgtccggccacactggaactgagacacggtccagacacctacggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgcc

SEQ ID NO: 547 - DNA, 250 bp, Bacteroides caccae
agagtttgatcctggctcaggatgaacgctagctacaggcttaacacatgcaagtcgaggggcatcagtttggtttgcttgcaaaccaaagctggcgaccggcgcacgggtgagtaacacgtatccaacctacctcatactcggggatagcctttcgaaagaaagattaatatccgatagcatatatttcccgcatgggttttatattaaagaaattcggtatgagatggggatgcgttccattagtttg

SEQ ID NO: 548 - DNA, 229 bp, Bacteroides caccae
agagtttgatcatggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatg

SEQ ID NO: 549 - DNA, 187 bp, Bacteroides caccae
agttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 550 - DNA, 253 bp, Bacteroides caccae
tacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 551 - DNA, 252 bp, Bacteroides caccae
tacggaaggtccgggcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacag

SEQ ID NO: 552 - DNA, 251 bp, Bacteroides caccae
tacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaggccaggggagcgaaag

SEQ ID NO: 553 - DNA, 253 bp, Bacteroides caccae; identical to SEQ ID NO: 550

SEQ ID NO: 554 - DNA, 120 bp, Bacteroides caccae
tgaggaatattggtcaatggacgcgagtctgaaccagccaagtagcgtgaaggatgactgccctatgggttgtaaacttcttttatatgggaataaagttgtccacgtgtggatttttgt

SEQ ID NO: 555 - DNA, 111 bp, Bacteroides caccae
cgaggaatattggtcaatggacgcgagtctgaaccagccaagtagcgtgaaggatgactgccctatgggttgtaaacttcttttatatgggaataaagttgtccacgtgtg

SEQ ID NO: 556 - DNA, 460 bp, Bacteroides caccae
cctacgggaggcagcagtgaggaatattggtcaatggacgcgagtctgaaccagccaagtagcgtgaaggatgactgccctatgggttgtaaacttcttttatatgggaataaagtggtccacgtgtggacttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtc

SEQ ID NO: 557 - DNA, 257 bp, Bacteroides caccae
gatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttggcggggtaacggccccaccaagac

SEQ ID NO: 558 - DNA, 237 bp, Bacteroides caccae
aagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagtggtccacgtgtggacttttgtatgtaccatatgaataaggatcggctaactcc

SEQ ID NO: 559 - DNA, 266 bp, Bacteroides caccae
gtttgtggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagtggtccacgtgtggacttttgtatgtaccatatgaataaggatcggctaactccgtgcc

SEQ ID NO: 560 - DNA, 301 bp, Porphyromonas sp.
agagtttgatcctggctcaggatgaacgctagcgataggcttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatactctccttagatcacatgagaagaggaggaaagattaatcgctaagagataggcctgcgttccattagctagttggtaaggtaacggcttaccaaggcaacgatggatagggggactgagaggttgac

SEQ ID NO: 561 - DNA, 229 bp, Porphyromonas sp.
agagtttgatcctggctcaggatgaacgctagcgataggcttaacacatgcaagtcgaggggcagcgagatgtagcaatacgtcgtcggcgaccggcgaatgggtgagtaacacgtatgcaacttacctcttagaggtgaataacccgatgaaagtcggactaatacaccatatactccttgggtcacatgaattgaggaggaaagatttatcgctaagagataggcct

SEQ ID NO: 562 - DNA, 229 bp, Porphyromonas sp.
agagtttgatcatggctcaggatgaacgctagcgataggcttaacacatgcaagtcgaggggcagcgagatgtagcaatacgtcgtcggcgaccggcgaatgggtgagtaacacgtatgcaacttacctcttagtggtgaataactcgatgaaagtcgaactaatacaccatactctccttagctcacatgagcagaggaggaaagattaatcgctaagagataggcct

SEQ ID NO: 563 - DNA, 177 bp, Porphyromonas sp.
tatgcgccttgccagcccgctcaggtgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactgga

SEQ ID NO: 564 - DNA, 253 bp, Porphyromonas sp.
tacggaggatgcgagcgttatccggaattattgggtttaaagggtgcgtaggttgcaagggaagtcaggggtgaaaagctatagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacagg

SEQ ID NO: 565 - DNA, 253 bp, Porphyromonas sp.
tacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacagg

SEQ ID NO: 566 - DNA, 253 bp, Porphyromonas sp.
tacggaggatgcgagcgttatccggaattattgggtttaaagggtgcgtaggttgcaagggaagtcaggggtgaaaagctatagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatcaaacagg

SEQ ID NO: 567 - DNA, 252 bp, Porphyromonas sp.
tacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatcaaacag

SEQ ID NO: 568 - DNA, 251 bp, Porphyromonas sp.
tacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatcaaaca

SEQ ID NO: 569 - DNA, 253 bp, Porphyromonas sp.; identical to SEQ ID NO: 564

SEQ ID NO: 570 - DNA, 253 bp, Porphyromonas sp.; identical to SEQ ID NO: 565

SEQ ID NO: 571 - DNA, 253 bp, Porphyromonas sp.; identical to SEQ ID NO: 566

SEQ ID NO: 572 - DNA, 120 bp, Porphyromonas sp.
tgaggaatattggtcaatgggcgagagcctgaaccagccaagtcgcgtgaaggaagactgcccgcaagggttgtaaacttcttttgtatgggattaaagtcgtctacgtgtagacgtttg

SEQ ID NO: 573 - DNA, 157 bp, Porphyromonas sp.
tgaggaatattggtcaatgggcgagagcctgaaccagccaagtcgcgtgaaggaagactgcccgcaagggttgtaaacttcttttgtatgggattaaagtcacctacgtgtaggtgttgcagttaccatacgaataagcatcggctaactccgtgcc

SEQ ID NO: 574 - DNA, 155 bp, Porphyromonas sp.
tgaggaatattggtcaatgggcgagagcctgaaccagccaagtcgcgtgaaggaagactgcccgcaagggttgtaaacttcttttgtatgggattaaagtcgtctacgtgtagacgtttgcagttaccatacgataagcatcggctaactccgtg

SEQ ID NO: 575 - DNA, 363 bp, Porphyromonas sp.
cctacgggtggctgcagtggggaattttgcacaatgggggaaaccctgatgcagcgacgccgcgtgatttagaaggccttcgggttgtaaaaatcttttctatgggaagaaaatgacagtaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgca

SEQ ID NO: 576 - DNA, 298 bp, Porphyromonas sp.
cctacgggaggctgcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtc

SEQ ID NO: 577 - DNA, 461 bp, Porphyromonas sp.
cctacgggaggcagcagtgaggaatattggtcaatgggcgagagcctgaaccagccaagtcgcgtgaaggaagactgcccgcaagggttgtaaacttcttttgtatgggattaaagtcgtctacgtgtaggcgtttgcagttaccatacgaataagcatcggctaactccgtgccagcagccgcggtaatgcggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtc

SEQ ID NO: 578 - DNA, 363 bp, Porphyromonas sp.
cctacggggggctgcagtgaggaatattggtcaatgggcgagagcctgaaccagccaagtcgcgtgaaggaagactgcccgcaagggttgtaaacttcttttgtatgggattaaagtcacctacgagtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatat

SEQ ID NO: 579 - DNA, 461 bp, Porphyromonas sp.
cctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtc

SEQ ID NO: 580 - DNA, 461 bp, Porphyromonas sp.
cctacgggtggcagcagtgaggaatattggtcaatgggcgagagcctgaaccagccaagtcgcgtgaaggaagactgcccgcaagggttgtaaacttcttttgtatgggattaaagtcgtctacgtgtagacgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatcaaacaggattagataccctggtagtc

SEQ ID NO: 581 - DNA, 257 bp, Porphyromonas sp.
taggtcacatgaagtagaggaggaaagatttaatcgctaagagataggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttt

SEQ ID NO: 582 - DNA, 315 bp, Porphyromonas sp.
tgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcgtctacgtgtaggcgtttgcagttaccatacgaataagcatcggctaactcc

SEQ ID NO: 583 - DNA, 170 bp, Porphyromonas sp.
cctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactcc

SEQ ID NO: 584 - DNA, 315 bp, Porphyromonas sp.
atgagaagaggaggaaagattaatcgctaagagatggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcgtctacgtgtagacgtttgcagttaccatacgaataagcatcggctaactcc

SEQ ID NO: 585 - DNA, 257 bp, Porphyromonas sp.
gatgaacgctagcgataggcttaacacatgcaagtcgaggggcagcgagatgtagcaatacgtcgtcggcgaccggcgaatgggtgagtaacacgtatgcaacttacctcttagaggtgaataacccgatgaaagtcggactaatacaccatatactccttgggtcacatgaattgaggaggaaagatttatcgctaagagataggcctgcgttccattagctcgttggtaaggtaacggcttaccaaggcaacgat

SEQ ID NO: 586 - DNA, 257 bp, Porphyromonas sp.
ttatggctcaggatgaacgctagcgataggcttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatactctccttagatcacatgagaagaggaggaaagattaatcgctaagagataggcctgcgttccattagctagttggtaaggtaacggcttacc

SEQ ID NO: 587 - DNA, 328 bp, Porphyromonas sp.
ttagatcacatgagaagaggaggaaagattatcgctaagagataggcctgcgttccattagctagttggtaagtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcgtctacgtgtagacgtttgcagttaccatacgaataagcatcggctaactccgtgcc

SEQ ID NO: 588 - DNA, 229 bp, Lactobacillus sp.
agagtttgatcatggctcaggacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaag

SEQ ID NO: 589 - DNA, 503 bp, Lactobacillus sp.
agagtttgatcatggctcaggatgaacgccggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatac

SEQ ID NO: 590 - DNA, 229 bp, Lactobacillus sp.
agagtttgatcatggctcaggacgaacgctggcggcgtgcctaatacatgcaagtcgagcgagcttgcctagatgaatttggtgcttgcaccaaatgaaactagatacaagcgagcggcggacgggtgagtaacacgtgggtaacctgcccaagagactgggataacacctggaaacagatgctaataccggataacaacactagacgcatgtctagagtttaaaagat

SEQ ID NO: 591 - DNA, 516 bp, Lactobacillus sp.
agagtttgatcctggctcaggatgaacgccggcggtgtgcctaatacatgcaagtcgaacgcgttgacccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaatggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtattt

SEQ ID NO: 592 - DNA, 372 bp, Lactobacillus sp.
agagtttgatcctggctcaggacgaacgctggcggcgtgcctaatacatgcaagtcgagcgagcttgcctagatgaatttggtgcttgcaccaaatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggag

SEQ ID NO: 593 - DNA, 229 bp, Lactobacillus sp.
agagtttgatcctggctcaggatgaacgccggcggtgtgcctaatacatgcaagtcgaacgcgttggcctaattgattgatggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaag

SEQ ID NO: 594 - DNA, 229 bp, Lactobacillus sp.
agagtttgatcatagctcaggacgaacgctggcggcgtgcctaatacatgcaagtcgagcgagctgaattcaaagatcccttcggggtgatttgttggatgctagcggcggatgggtgagtaacacgtgggcaatctgccctaaagactgggataccacttggaaacaggtgctaataccggataacaacatgaatcgcatgattcaagtttgaaaggcggcgtaagct

SEQ ID NO: 595 - DNA, 229 bp, Lactobacillus sp.
agagtttgatcctggctcaggatgaacgctggcggcgtgcctaatacatgcaagtcgaacgaaacttctttatcaccgagtgcttgcactcgccgataaagagttgagtggcgaacgggtgagtaacacgtgggcaacctgcccaaaagagggggataacacttggaaacaggtgctaataccgcataaccatagttaccgcatggtaactatgtaaaaggtggctatg

SEQ ID NO: 596 - DNA, 229 bp, Lactobacillus sp.
agagtttgatcatggctcaggacgaacgctggcggcgtgcctaatacatgcaagtcgagcgagcggaaccaacagatttacttcggtaatgacgttgggaaagcgagcggcggatgggtgagtaacacgtggggaacctgcccctaagtctgggataccatttggaaacaggtgctaataccggataataaagcagatcgcatgatcagcttttgaaaggcggcgtaag

SEQ ID NO: 597 - DNA, 252 bp, Lactobacillus sp.
tacgtaggtggcaagcgttgtccggatttattgggcgtaaagagaatgtaggcggtttattaagtttgaagtgaaagccctcggctcaaccgaggaagtgcttcgaaaactggtaaacttgaatgcagaagaggaaagtggaactccatgtgtagcggtggaatgcgtagatatatggaagaacaccagtggcgaaggcggctttctggtctgtaactgacgctgagattcgaaagcatgggtagcaaacag

SEQ ID NO: 598 - DNA, 253 bp, Lactobacillus sp.
tacgtaggtggcaagcgttatccggatttattgggcgtaaagcgagcgcaggcggttgcttaggtctgatgtgaaagccttcggcttaaccgaagaagggcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacagg

SEQ ID NO: 599 - DNA, 252 bp, Lactobacillus sp.
tacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacag

SEQ ID NO: 600 - DNA, 253 bp, Lactobacillus sp.
tacgtaggtggcaagcgttgtccggatttattgggcgtaaagcgagcgcaggcggaaagataagtctgatgtgaaagccctcggctcaaccgaggaattgcatcggaaactgtgtttcttgagtgcagaagaggagagtggaactccatgtgtagcggtggaatgcgtagatatatggaagaacaccagtggcgaaggcggctctctggtctgtaactgacgctgaggctcgaaagcatgggtagcgaacagg

SEQ ID NO: 601 - DNA, 252 bp, Lactobacillus sp.
tacgtaggtggcaagcgttatccggatttattgggcgtaaagggaacgcaggcggtcttttaagtctgatgtgaaagccttcggcttaaccggagtagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacag

SEQ ID NO: 602 - DNA, 251 bp, Lactobacillus sp.
tacgtaggtggcaagcgttgtccggatttattgggcgtaaagagaatgtaggcggtttattaagtttgaagtgaaagccctcggctcaaccgaggaagtgcttcgaaaactggtaaacttgagtgcagaagaggaaagtggaactccatgtgtagcggtggaatgcgtagatatatggaagaacaccagtggcgaaggcggctttctggtctgtaactgacgctgagattcgaaagcatgggtagcaaaca

SEQ ID NO: 603 - DNA, 251 bp, Lactobacillus sp.
tacgtaggtggcaagcgttatccggatttattgggcgtaaagcgagcgcaggcggttgcttaggtctgatgtgaaagccttcggcttaaccgaagaagtgcatcggaaaccgggcgacttgagtacaggagaggtaagtggaattcctagtgtagcggtgaaatgcgtagatattaggaggaacaccagtggcgaaggcggctctctggtctgcaactgacgctgaggctcgaaagcatgggtagcgaaca

SEQ ID NO: 604 - DNA, 251 bp, Lactobacillus sp.
tacgtagggagcaagcgttgtccggatttattgggcgtaaagcgagcgcaggcggtttcttaggtctgatgtgaaagccttcggcttaaccggagaagtgcatcggaaaccaggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaaca

SEQ ID NO: 605 - DNA, 251 bp, Lactobacillus sp.
tacgtaggtggcaagcgttatccggatttattgggcgtaaagcgagcgcaggcggttgcttaggtctgatgtgaaagccttcggcttaaccgaagaagtgcatcggaaaccgggcgacttgagtggagtagaggcaagcggaattccgagtgtagcggtgaaatgcgtagatatatggaagaacaccagtggcgaaggcggctctctggtctgcaactgacgctgaggctcgaaagcatgggtagcgaaca

SEQ ID NO: 606 - DNA, 251 bp, Lactobacillus sp.
tacgtaggtggcaagcgttgtccggatttattgggcgtaaagcgagcgcaggcggaagaataagtctgatgtgaaagccctcggcttaaccgaggaactgcatcggaaactgtttttcttgagtatcggagaggcaatcggaattcctagtgtagcggtgaaatgcgtagatatatggaagaacaccagtggcgaaggcggctgtctggtctgcaactgacgctgaggctcgaaagcatgggtagcgaaca

SEQ ID NO: 607 - DNA, 251 bp, Lactobacillus sp.
tacgtaggtggcaagcgttgtccggatttattgggcgtaaagcgagcgcaggcggattgataagtctgatgtgaaagccttcggctcaaccgaagaactgcatcagaaactgtcaatcttgagtgcagaagaggagagtggaactccatgtgtagcggtggaatgcgtagatatatggaagaacaccagtggcgaaggcggctctctggtctgtaactgacgctgaggctcgaaagcatgggtagcgaaca

SEQ ID NO: 608 - DNA, 251 bp, Lactobacillus sp.
tacgtagggggcaagcgttatccggatttattgggcgtaaagcgagcgcaggcggaagaataagtctgatgtgaaagccttcggctcaaccggagaattgcatcagaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaaca

SEQ ID NO: 609 - DNA, 251 bp, Lactobacillus sp.
tacagaggtctcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggcacgaaagcgtggggagcaaaca

SEQ ID NO: 610 - DNA, 251 bp, Lactobacillus sp.
tacgtaggtggcaagcgttgtccggatttattgggcgtaaagcgagtgcaggcggttgcttaggtctgatgtgaaagccttcggcttaaccgaagaagtgcatcggaaactgtttttcttgagtgcagaagaggagagtggaactccatgtgtagcggtggaatgcgtagatatatggaagaacaccagtggcgaaggcggctgtctggtctgcaactgacgctgaggctcgaaagcatgggtagcgaaca

SEQ ID NO: 611 - DNA, 251 bp, Lactobacillus sp.
tacgtaggtggcaagcgttgtccggatttattgggcgtaaagcgagcgcaggcggaagaataagtctgatgtgaaagccctcggcttaaccgaggaactgcattggaaactgccagtcttgagtgccggagaggtaagcggaattcctagtgtagcggtgaaatgcgtagatattaggaggaacaccagtggcgaaggcggctgtctggtctgcaactgacgctgaggctcgaaagcatgggtagcgaaca

SEQ ID NO: 612 - DNA, 251 bp, Lactobacillus sp.
tacgtaggtggcaagcgttgtccggatttattgggcgtaaagcgagcgcaggcggaagaataagtctgatgtgaaagccctcggcttaaccgaggaactgcatcggaaactgtcgaactagagtgtcggagaggcaagtggaattcctagtgtagcggtgaaatgcgtagatattaggaggaacaccagtggcgaaggcggctgtctggtctgcaactgacgctgaggctcgaaagcatgggtagcgaaca

SEQ ID NO: 613 - DNA, 251 bp, Lactobacillus sp.
tacgtaggtcccgagcgttatccggatttattgggcgtaaagggaacgcaggcggtcttttaagtctgatgtgaaagccttcggcttaaccggagtagtgcattggaaactggaagacttgagtgcagaaaaggagagtggaactccatgtgtagcggtgaaatgcgtagatattaggaggaacaccagtggcgaaagcggctctctggtctgtaactgacgctgaggttcgaaagcgtgggtagcaaaca

SEQ ID NO: 614 - DNA, 251 bp, Lactobacillus sp.
tacgtaggtggcaagcgttgtccggatttattgggcgtaaagcgagcgcaggcggaagaataagtctgatgtgaaagccctcggcttaaccgaggaactgcatcggaaactgttgaacttgagtgcagaagaggagagtggaattccatgtgtagcggtgaaatgcgtagatatatggaagaacaccagtggcgaaggcggctctctggtctgcaactgacggtgaggctcgaaagcatggggagcaaaca

SEQ ID NO: 615 - DNA, 251 bp, Lactobacillus sp.
tacgtaggtggcaagcgttatccggatttattgggcgtaaagcgagcgcaggcggttgcttaggtctgatgtgaaagccttcggcttaaccgaagaagtgcatcggaaaccgggcgacttgagtgaagtagaggcaggcggaattccccgtgtagcggtgaaatgcgtagagatggggaggaacaccagtggcgaaggcggctgtctggtctgcaactgacgctgaggctcgaaagcatgggtagcgaaca

SEQ ID NO: 616 - DNA, 251 bp, Lactobacillus sp.
tacgtaggtggcaagcgttgtccggatttattgggcgtaaagggaacgcaggcggtcttttaagtctgatgtgaaagccttcggcttaaccggagtagtgcattggaaactgtcaaacttgagtgcagaaggggagagtggaattccatgtgtagcggtgaaatgcgtagatatatggaggaacaccggtggcgaaagcggctctctggtctgtaactgacgctgaggttcgaaagcgtgggtagcaaaca

SEQ ID NO: 617 - DNA, 251 bp, Lactobacillus sp.
tacgtagggagcgagcgttatccggatttattgggcgtaaagcgagcgcaggcggaagaataagtctgatgtgaaagccctcggcttaaccgaggaactgcatcggaaactgggcgacttgagtgcagaagaggagagtggaactccatgtgtagcggtggaatgcgtagatatatggaagaacaccagtggcgaaggcggctctctggtctgcaactgacgctgaggctcgaaagcatgggtagcgaaca

SEQ ID NO: 618 - DNA, 253 bp, Lactobacillus sp.
tacgtaggtggcaagcgttgtccggatttattgggcgtaaagcgagcgcaggcggaaaaataagtctaatgtgaaagccctcggcttaaccgaggaattgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacagg

SEQ ID NO: 619 - DNA, 253 bp, Lactobacillus sp.; identical to SEQ ID NO: 598

SEQ ID NO: 620 - DNA, 253 bp, Lactobacillus sp.
tacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtggggagcgaacagg

SEQ ID NO: 621 - DNA, 253 bp, Lactobacillus sp.
tacgtaggtggcaagcgttgtccggatttattgggcgtaaagcgagcgcaggcggttttttaagtctgatgtgaaagccttcggcttaaccgaagaagtgcattagaaactggaaaacttgagtgcagaagaggacagtggaactccatgtgtagcggtgaaatgcgtagatatatggaagaacaccagtggcgaaggcggctgtctggtctgtaactgacgctgaggctcgaaagtatggggagcgaacagg

SEQ ID NO: 622 - DNA, 253 bp, Lactobacillus sp.
tacgtaggtggcaagcgttgtccggatttattgggcgtaaagcgagcgcaggcggttcaataagtctgatgtgaaagccctcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacagg

SEQ ID NO: 623 - DNA, 253 bp, Lactobacillus sp.
tacgtaggtggcaagcgttatccggatttattgggcgtaaagcgagcgcaggcggttttttaagtctgatgtgaaagccctcggcttaaccgaggaattgcatcggaaactgggaaacttgagtgcagaagaggaaagtggaactccatgtgtagcggtgaaatgcgtagatatatggaagaacaccagtggcgaaggcggctgtctggtctgtaactgacgctgaggctcgaaagcatgggtagcgaacagg

SEQ ID NO: 624 - DNA, 253 bp, Lactobacillus sp.
tacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcgactgtctggtctgcaactgacgctgaggctcgaaagcatgggtagcgaacagg

SEQ ID NO: 625 - DNA, 251 bp, Lactobacillus sp.
tacgtaggtggcaagcgttatccggatttattgggcgtaaagggaacgcaggcggttctttaagtctgatgtgaaagccttcggcttaaccgaagatgtgcattggaaactggggaacttgagtgcagaagaggagagtggaactccatgtgtagcggtgaaatgcgtagatatatggaagaacaccagtggcgaaagcggctctctggtctgtaactgacgctgaggttcgaaagcgtgggtagcgaaca

SEQ ID NO: 626 - DNA, 120 bp, Lactobacillus sp.
tagggaatcttccacaatggacgcaagtctgatggagcaacgccgcgtgagtgaagaaggttttcggatcgtaaagctctgttgttggtgaagaaggatagaggtagtaactggccttta

SEQ ID NO: 627 - DNA, 111 bp, Lactobacillus sp.
cgagagataccctacgggagcagcagtaggggaatcttccacaatggacgcaagtctgatggagcaacgccgcgtgagtgaagaaggttttcggatcgtaaagctctgttg

SEQ ID NO: 628 - DNA, 94 bp, Lactobacillus sp.
ctacgtaaaactctgttgttagagaagaacagccgtgagagcaactgctcatggtatgacggtatctaaccagaaagtcacggctaactacgtg

SEQ ID NO: 629 - DNA, 111 bp, Lactobacillus sp.
tagggaatcttccacatggacgaaagtctgatggagcaacgccgcgtgagtgaagaagggtttcggctcgtaaaactctgttgttagagaagaacagccgtgagagcaact

SEQ ID NO: 630 - DNA, 111 bp, Lactobacillus sp.
tagggaatcttccacatggacgcaagtctgatggagcaacgccgcgtgagtgaagaaggttttcggatcgtaaagctctgttgttggtgaagaaggatagaggcagtaact

SEQ ID NO: 631 - DNA, 465 bp, Lactobacillus sp.
cctacggggggctgcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcagtaatacgtaggtggcaagcgttgtccggatttattgggcgtaaagcgagcgcaggcggaaaaataagtctaatgtgaaagccctcggcttaaccgaggaattgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtc

SEQ ID NO: 632 - DNA, 465 bp, Lactobacillus sp.
cctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaacccgaaagtcacggctaactacgtgccagcagccgcggtaatacggaggtggcaagcgttatccggatttattgggcgtaaagcgagcgcaggcgggtgcttaggtctgatgtgaaagccttcggcttaaccgaagaagggcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtc

SEQ ID NO: 633 - DNA, 465 bp, Lactobacillus sp.
  misc_feature (2)..(2): n is a, c, g, or t
cntacgggtggcagcagtagggaatcttccacaatgggcgcaagcctgatggagcaacaccgcgtgagtgaagaagggcttcggctcgtaaagctctgttgttggagaagaacgtgcgtaagagtaactgtttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtc

SEQ ID NO: 634 - DNA, 465 bp, Lactobacillus sp.
cctacgggaggctgcagtagggaatcttccacaatgggcgcaagcctgatggagcaacaccgcgtgagtgaagaagggtttcggctcgtaaagctctgttgttggagaagaatatgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcgactgtctggtctgcaactgacgctgaggctcgaaagcatgggtagcgaacaggattagataccctggtagtc

SEQ ID NO: 635 - DNA, 464 bp, Lactobacillus sp.
cctacggggggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtagggggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctgtagtc

SEQ ID NO: 636 - DNA, 465 bp, Lactobacillus sp.
cctacggggggcagcagtagggaatcttccacaatggacgcaagtctgatggagcaacgccgcgtgagtgaagaagggtttcggctcgtaaagctctgttgttggtgaagaaggacaggggtagtaactgacctttgtttgacggtaatcaattagaaagtcacggctaactacgtgccagcagccgcggtaatacgtaggtggcaagcgttgtccggatttattgggcgtaaagcgagtgcaggcggttcgataagtctgatgtgaaagccttcggctcaaccggagaattgcatcagaaactgtcgagcttgagtacagaagaggagagtggaactccatgtgtagcggtgaaatgcgtagatatatggaagaacaccggtggcgaaggcggctctctggtctgttactgacgctgaggctcgaaagcatgggtagcgaacaggattagataccccagtagtc

SEQ ID NO: 637 - DNA, 363 bp, Lactobacillus sp.
cctacgggtggctgcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtag

SEQ ID NO: 638 - DNA, 363 bp, Lactobacillus sp.
cctacgggtggctgcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtag

SEQ ID NO: 639 - DNA, 363 bp, Lactobacillus sp.
cctacgggaggctgcagtagggaatcttccacaatggacgaaagtctgatggagcaacgccgcgtgagtgaagaaggttttaggatcgtaaaactctgttgttggagaagaacagggactagagtaactgttagtcctttgacggtatccaaccagaaagccacggctaactacgtgccagcagccgcggtaatacgtaggtggcaagcgttgtccggatttattgggcgtaaagcgagcgcaggcggaccggcaagttggaagtgaaaactatgggctcaacccataaattgctttcaaaactgtttttcttgagtagtgcagaggtaggcggaattcccggtgtagcggtggaatgcgtag

SEQ ID NO: 640 - DNA, 204 bp, Lactobacillus sp.
ccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactac

SEQ ID NO: 641 - DNA, 397 bp, Parvimonas micra; identical to SEQ ID NO: 26

SEQ ID NO: 642 - DNA, 149 bp, Parvimonas micra
gaaactggaagacttgagtgaaggagaggaaagtggaattcctagtgtagcggtgaaatgcgtagatattaggaggaataccggtggcgaaggcgactttctggtacttttactgacgctcaggtacgaaagcgtggggagcaaacagg

SEQ ID NO: 643 - DNA, 252 bp, Parvimonas micra; identical to SEQ ID NO: 29

SEQ ID NO: 644 - DNA, 252 bp, Parvimonas micra; identical to SEQ ID NO: 29

SEQ ID NO: 645 - DNA, 438 bp, Parvimonas micra; identical to SEQ ID NO: 36

SEQ ID NO: 646 - DNA, 460 bp, Parvimonas micra; identical to SEQ ID NO: 40

SEQ ID NO: 647 - DNA, 462 bp, Parvimonas micra; identical to SEQ ID NO: 42

SEQ ID NO: 648 - DNA, 252 bp, Peptostreptococcus stomatis
tacgtagggggctagcgttatccggatttactgggcgtaaagggtgcgtaggtggtccttcaagtcggtggttaaaggctacggctcaaccgtagtaagccgccgaaactggaggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacagg

SEQ ID NO: 649 - DNA, 252 bp, Peptostreptococcus stomatis; identical to SEQ ID NO: 648

SEQ ID NO: 650 - DNA, 137 bp, Peptostreptococcus stomatis
tggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgcc

SEQ ID NO: 651 - DNA, 135 bp, Peptostreptococcus stomatis
tggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtg

SEQ ID NO: 652 - DNA, 439 bp, Peptostreptococcus stomatis
cctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagccgccgaaactggaggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtc

SEQ ID NO: 653 - DNA, 311 bp, Peptostreptococcus stomatis
aatatatatttgcggcatcgcagatatatcaaagtgttagcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactac

SEQ ID NO: 654 - DNA, 247 bp, Peptostreptococcus stomatis
ctagttggtgagataactgcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgcc

SEQ ID NO: 655 - DNA, 253 bp, Porphyromonas asaccharolytica; identical to SEQ ID NO: 565

SEQ ID NO: 656 - DNA, 253 bp, Porphyromonas asaccharolytica; identical to SEQ ID NO: 565

SEQ ID NO: 657 - DNA, 157 bp, Porphyromonas asaccharolytica; identical to SEQ ID NO: 573

SEQ ID NO: 658 - DNA, 156 bp, Porphyromonas asaccharolytica
tgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtg

SEQ ID NO: 659 - DNA, 461 bp, Porphyromonas asaccharolytica; identical to SEQ ID NO: 579

SEQ ID NO: 660 - DNA, 170 bp, Porphyromonas asaccharolytica; identical to SEQ ID NO: 583

SEQ ID NO: 661 - DNA, 20 bp, Artificial Sequence
  Synthetically constructed PCR primer 27F
agagtttgatcctggctcag

SEQ ID NO: 662 - DNA, 19 bp, Artificial Sequence
  Synthetically constructed PCR primer 533R
ttaccgcggctgctggcac

SEQ ID NO: 663 - DNA, 25 bp, Artificial Sequence
  Synthetically constructed 16S rRNA bacterial universal PCR forward primer
  misc_feature (1)..(8): n is a, c, g, or t
nnnnnnnncctacgggaggcagcag

SEQ ID NO: 664 - DNA, 23 bp, Artificial Sequence
  Synthetically constructed 16S rRNA bacterial universal PCR reverse primer
  misc_feature (1)..(8): n is a, c, g, or t
nnnnnnnnattaccgcggctgct

SEQ ID NO: 665 - DNA, 15 bp, Artificial Sequence
  Synthetically constructed V3F forward PCR primer
tacggraggcagcag

SEQ ID NO: 666 - DNA, 20 bp, Artificial Sequence
  Synthetically constructed V4R reverse PCR primer
ggactaccagggtatctaat